(19) United States
(12) Patent Application Publication
(10) Pub. No.: US 2010/0261679 A1
(43) Pub. Date: Oct. 14, 2010

(54) CSF-1R, Inhibitors, Compositions, and Methods of Use

(76) Inventors: James Sutton, Pleasanton, CA (US); Martin Sendzik, San Mateo, CA (US); Weibo Wang, Moraga, CA (US)

Correspondence Address: NOVARTIS INSTITUTES FOR BIOMEDICAL RESEARCH, INC., 220 MASSACHUSETTS AVENUE, CAMBRIDGE, MA, 02139, US

(21) Appl. No.: 12/738,277

(22) PCT Filed: Oct. 16, 2008
(86) PCT No.: PCT/EP08/63952

§ 371 Date: Apr. 15, 2010

Related U.S. Application Data
(60) Provisional application No. 60/981,058, filed on Oct. 18, 2007.

(51) Int. Cl. A61K 31/66; C12N 5/071; A61K 31/4709; A61K 31/517; A61K 31/5377; A61K 31/4439; C07D 215/38; C07D 401/12; C07D 413/12; C07D 417/10; A61P 35/00

(52) U.S. Cl. 514/102; 435/325; 514/313; 514/266.21; 514/234.5; 514/338; 546/162; 544/284; 544/124; 546/270.1

(57) ABSTRACT

Disclosed herein are compounds and their oxides, esters, prodrugs, solvates, and pharmaceutically acceptable salts thereof, compositions of the compounds, either alone or in combination with at least one additional therapeutic agent, with a pharmaceutically acceptable carrier, and uses of the compounds, either alone or in combination with at least one additional therapeutic agent. The embodiments are useful for inhibiting cellular proliferation, inhibiting the growth and/or metathesis of tumors, treating or preventing cancer, treating or preventing degenerating bone diseases such as rheumatoid arthritis, and/or inhibiting molecules such as CSF-1R.

This application is a U.S. National Phase filing of International Application No. PCT/EP2008/063952, filed 16 Oct. 2008, and claims priority to U.S. provisional application Ser. No. 60/981,058, filed 18 Oct. 2007, the contents of which are incorporated herein by reference in their entirety.

### BACKGROUND OF THE INVENTION

1. Field of the Invention

The present invention relates to CSF-1R inhibitory compounds, their oxides, esters, prodrugs, solvates, or pharmaceutically acceptable salts thereof. This invention also relates to compositions of the compounds together with pharmaceutically acceptable carriers. In another aspect, this invention relates to uses of the compounds, either alone or in combination with at least one additional therapeutic agent, in the prophylaxis or treatment of cancer and in other CSF-1R mediated diseases.

2. State of the Art

CSF-1R is the receptor for M-CSF (macrophage colony stimulating factor, also called CSF-1) and mediates the biological effects of this cytokine (Sherr 1985). The cloning of the colony stimulating factor-1 receptor (also called c-fins) was described for the first time in Roussel et al., Nature 325:549-552 (1987). In that publication, it was shown that CSF-1R had transforming potential dependent on changes in the C-terminal tail of the protein including the loss of the inhibitory tyrosine 969 phosphorylation which binds Cbl and thereby regulates receptor down regulation (Lee 1999).

CSF-1R is a single chain, transmembrane receptor tyrosine kinase (RTK) and a member of the family of immunoglobulin (Ig) motif containing RTKs characterized by repeated Ig domains in the extracellular portion of the receptor. The intracellular protein tyrosine kinase domain is interrupted by a unique insert domain that is also present in the other related RTK class III family members that include the platelet derived growth factor receptors (PDGFR), stem cell growth factor receptor (c-Kit) and fms-like cytokine receptor (FLT3). In spite of the structural homology among this family of growth factor receptors, they have distinct tissue-specific functions. CSF-1R is mainly expressed on cells of the monocytic lineage and in the female reproductive tract and placenta. In addition, expression of CSF-1R has been reported in Langerhans cells in skin, a subset of smooth muscle cells (Inaba 1992), B cells (Baker 1993) and microglia (Sawada 1990).

The main biological effects of CSF-1R signaling are the differentiation, proliferation, migration, and survival of the precursor macrophages and osteoclasts from the monocytic lineage. Activation of CSF-1R is mediated by its only ligand, M-CSF. Binding of M-CSF to CSF-1R induces the formation of homodimers and activation of the kinase by tyrosine phosphorylation (Stanley 1997). Further signaling is mediated by the p85 subunit of PI3K and Grb2 connecting to the PI3K/AKT and Ras/MAPK pathways, respectively. These two important signaling pathways can regulate proliferation, survival and apoptosis. Other signaling molecules that bind the phosphorylated intracellular domain of CSF-1R include STAT1, STAT3, PLCγ, and Cbl (Bourette 2000).

CSF-1R signaling has a physiological role in immune responses, in bone remodeling and in the reproductive system. The knockout animals for either M-CSF-1 (op/op mouse; Pollard 1996) or CSF-1R (Dai 2002) have been shown to have osteopetrotic, hematopoietic, tissue macrophage, and reproductive phenotypes consistent with a role for CSF-1R in the respective cell types.

The recent success of Herceptin® and Avastin® has underscored the importance in developing therapeutics targeting a specific biological target. These drugs can minimize adverse events, have greater predictability, give physicians greater flexibility in their treatments, and provide researchers with a better understanding of a particular target. Additionally, targeted therapy may allow treatment of multiple indications affected by the same signaling pathway with fewer and potentially easier to manage toxicities. (BioCentury, V. 14(10) February, 2006) Inhibition of an individual kinase, such as CSF-1R, which is integrated within a pathway associated with cancer or other diseases, can effectively modulate downstream kinases as well, thereby affecting the entire pathway. However, the active sites of 491 human protein kinase domains are highly conserved, which makes the design of selective inhibitors a formidable challenge (Cohen 2005). Accordingly, there is a need for selective kinase inhibitors, such as selective CSF-1R inhibitors.

### SUMMARY OF THE INVENTION

A continuing need exists for compounds that inhibit cellular proliferation, inhibit the growth of tumors, treat cancer, modulate cell cycle arrest, and/or specifically inhibit molecules such as CSF-1R, and for pharmaceutical formulations and medicaments that contain such compounds. A need also exists for selective CSF-1R inhibitory compounds. A need also exists for methods of administering such compounds, pharmaceutical formulations, and medicaments to patients or subjects in need thereof.

In some embodiments, the present invention is directed to a method for treating a CSF-1R mediated disorder in a patient, comprising administering to the patient a compound of Formula (I):

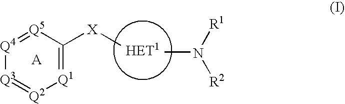

or an oxide, ester, prodrug, pharmaceutically acceptable salt, or solvate thereof, wherein:

A is a six-member ring where each of Q¹, Q², Q³, Q⁴ and Q⁵ is independently C—R³ or N, provided that at least one of Q¹, Q², Q³, Q⁴ and Q⁵ is N and at most three of Q¹, Q², Q³, Q⁴ and Q⁵ are N;

each R³ is independently hydrogen or R^{3a}, where R^{3a} is selected from the group consisting of halo, alkyl, substituted alkyl, alkenyl, substituted alkenyl, alkynyl, substituted alkynyl, carbonitrile, aryl, substituted aryl, cycloalkyl, substituted cycloalkyl, cycloalkenyl, substituted cycloalkenyl, heteroaryl, substituted heteroaryl, heterocyclic, substituted heterocyclic, amino, substituted amino, acylamino, alkoxy, substituted alkoxy, carboxyl, carboxyl ester, substituted sulfonyl, aminosulfonyl, and aminocarbonyl; or two adjacent R^{3a} groups together form a aryl, substituted aryl, heterocyclic, substituted heterocyclic, heteroaryl, or substituted heteroaryl group that is fused to ring A;

HET¹ is a bicyclic ring selected from the group consisting of:
(a) a [6,6] fused bicyclic ring selected from the group consisting of:

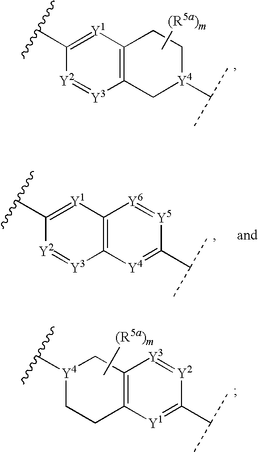

(b) a [5,6] fused bicyclic ring selected from the group consisting of:

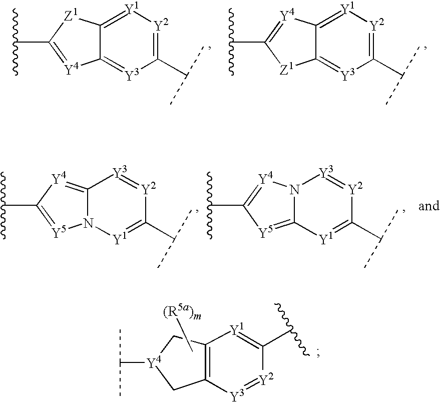

(c) a [6,5] fused bicyclic rink selected from the group consisting of:

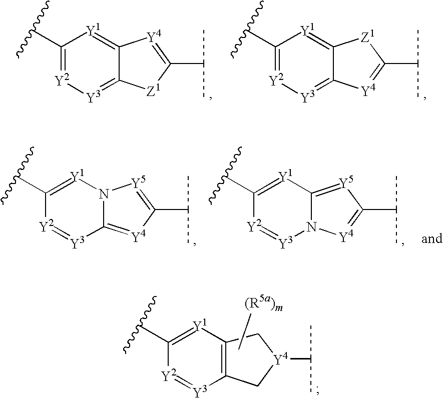

and
(d) a [5,5] fused bicyclic ring selected from the group consisting of:

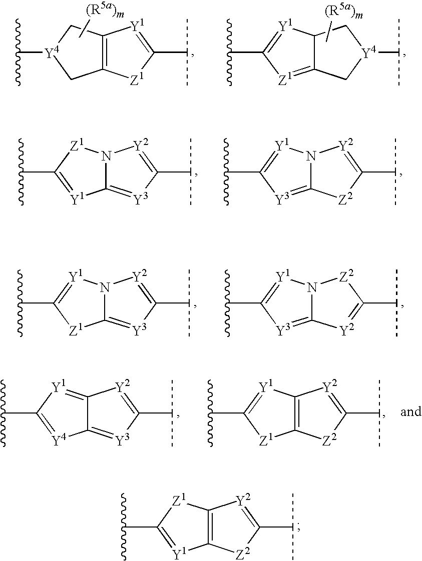

wherein the wavy line represents point of connection with X and dashed line represents point of connection with —NR¹R²;

R¹ and R² are independently selected from the group consisting of hydrogen, alkyl, substituted alkyl, cycloalkyl, substituted cycloalkyl, cycloalkenyl, substituted cycloalkenyl, aryl, substituted aryl, heterocyclyl, substituted heterocyclyl, heteroaryl, substituted heteroaryl, acyl, and aminocarbonyl, or R¹ and R² together with the nitrogen atom bond thereof form a group selected from heterocyclyl, substituted heterocyclyl, heteroaryl, and substituted heteroaryl; provided R¹ and R² are not both hydrogen;

Y¹, Y², Y³, Y⁴, Y⁵, and Y⁶ are independently selected from the group consisting of C—R⁵ and N; where each R⁵ is independently hydrogen or R^{5a};

R^{5a} is independently selected from the group consisting of alkyl, substituted alkyl, alkoxy, substituted alkoxy, amino, substituted amino, and halo, or optionally when m is at least 2, two R^{5a} together with the carbon atom to which they are both attached from a C═O or C═S group;

m is 0, 1, 2, 3, 4, or 5;

Z¹ and Z² are independently selected from the group consisting of C(—R⁵)₂, O, N—R⁶, S, and S(O); where each R⁶ is independently selected from the group consisting of hydrogen, alkyl, and substituted alkyl; and

X is selected from the group consisting of O, S, S(O), S(O)₂, and N—R⁴, wherein R⁴ is hydrogen, alkyl, or substituted alkyl; provided that when X is O, HET¹ is not

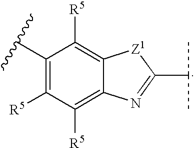

In some embodiments of its compound aspect, the present invention is directed to compounds of Formula (II):

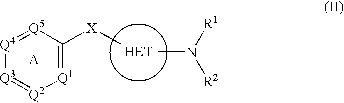

or an oxide, ester, prodrug, pharmaceutically acceptable salt, or solvate thereof, wherein:

A is a six-member ring where each of Q¹, Q², Q³, Q⁴ and Q⁵ is independently C—R³ or N, provided that at least one of Q¹, Q², Q³, Q⁴ and Q⁵ is N and at most three of Q¹, Q², Q³, Q⁴ and Q⁵ are N;

each R³ is independently hydrogen or R^{3a}, where R^{3a} is selected from the group consisting of halo, alkyl, substituted alkyl, alkenyl, substituted alkenyl, alkynyl, substituted alkynyl, carbonitrile, aryl, substituted aryl, cycloalkyl, substituted cycloalkyl, cycloalkenyl, substituted cycloalkenyl, heteroaryl, substituted heteroaryl, heterocyclic, substituted heterocyclic, amino, substituted amino, acylamino, alkoxy, substituted alkoxy, carboxyl, carboxyl ester, substituted sulfonyl, aminosulfonyl, and aminocarbonyl; or two adjacent R^{3a} groups together form a aryl, substituted aryl, heterocyclic, substituted heterocyclic, heteroaryl, or substituted heteroaryl group that is fused to ring A;

HET is a bicyclic ring selected from the group consisting of:
(a) a [6,6] fused bicyclic ring selected from the group consisting of:

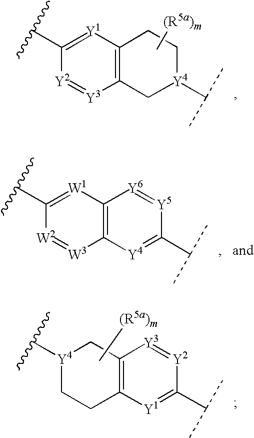

(b) a [5,6] fused bicyclic ring selected from the group consisting of:

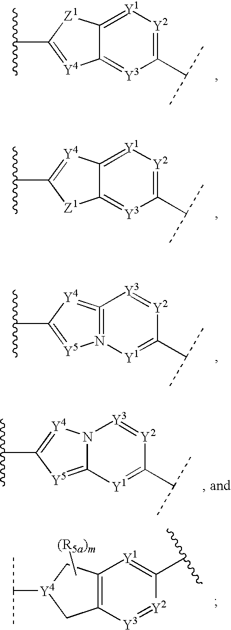

(c) a [6,5] fused bicyclic ring selected from the group consisting of:

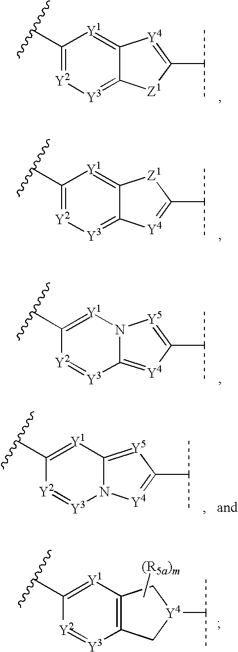

and
(d) a [5,5] fused bicyclic ring selected from the group consisting of:

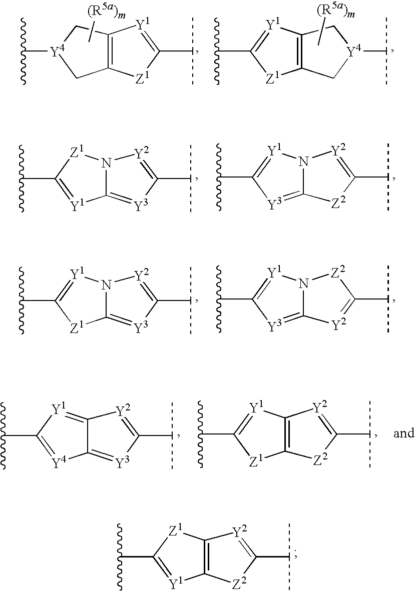

wherein the wavy line represents point of connection with X and dashed line represents point of connection with —NR¹R²;

R¹ and R² are independently selected from the group consisting of hydrogen, alkyl, substituted alkyl, cycloalkyl, substituted cycloalkyl, cycloalkenyl, substituted cycloalkenyl, aryl, substituted aryl, heterocyclyl, substituted heterocyclyl, heteroaryl, substituted heteroaryl, acyl, and aminocarbonyl, or R¹ and R² are taken together to form a group selected from heterocyclyl, substituted heterocyclyl, heteroaryl, and substituted heteroaryl; provided R¹ and R² are not both hydrogen;

Y¹, Y², Y³, Y⁴, Y⁵, and Y⁶ are independently selected from the group consisting of C—R⁵ and N;

W¹, W², and W³ are independently selected from the group consisting of C—R⁵ and N, provided that at least one of W¹, W², and W³ is N;

each R⁵ is independently hydrogen or R^{5a};

R^{5a} is independently selected from the group consisting of alkyl, substituted alkyl, alkoxy, substituted alkoxy, amino, substituted amino, and halo, or optionally when m is at least 2, two R^{5a} together with the carbon atom to which they are both attached from a C═O or C═S group;

m is 0, 1, 2, 3, 4, or 5;

Z¹ and Z² are independently selected from the group consisting of C(—R⁵)₂, O, N—R⁶, S, and S(O); where each R⁶ is independently selected from the group consisting of hydrogen, alkyl, and substituted alkyl; and

X is selected from the group consisting of O, S, S(O), S(O)₂, and N—R⁴, wherein R⁴ is hydrogen, alkyl, or substituted alkyl; provided that when X is O, HET is not

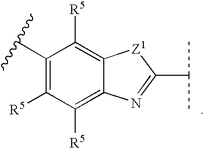

In some embodiments of its compound aspect, the present invention is directed to compounds of Formula (VII):

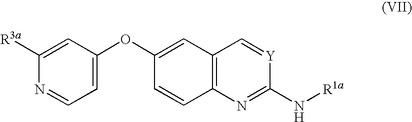

or an oxide, ester, prodrug, pharmaceutically acceptable salt, or solvate thereof,

wherein:

Y is N or CH;

R^{1a} is selected from the group consisting of hydrogen, alkyl, substituted alkyl, cycloalkyl, substituted cycloalkyl, cycloalkenyl, substituted cycloalkenyl, heterocyclyl, substituted heterocyclyl, acyl, and aminocarbonyl; and

R^{3a} is selected from the group consisting of halo, alkyl, substituted alkyl, alkenyl, substituted alkenyl, alkynyl, substituted alkynyl, carbonitrile, aryl, substituted aryl, cycloalkyl, substituted cycloalkyl, cycloalkenyl, substituted cycloalkenyl, heteroaryl, substituted heteroaryl, heterocyclic, substituted heterocyclic, amino, substituted amino, acylamino, alkoxy, substituted alkoxy, carboxyl, carboxyl ester, substituted sulfonyl, aminosulfonyl, and aminocarbonyl;

provided that R^{1a} is not:

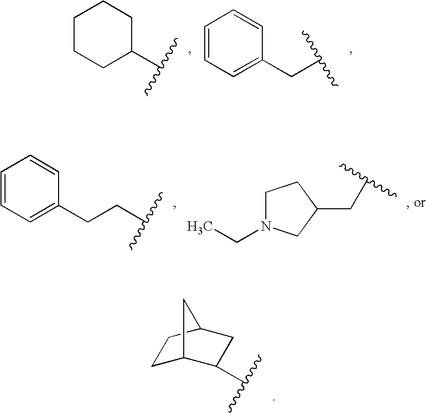

In other embodiments of its compound aspect, the present invention is directed to any one of the compounds in Tables 1-4 below.

These and other embodiments of the invention are further described in the Detailed Description that follows.

### DETAILED DESCRIPTION

Throughout this application, the text refers to various embodiments relating to compounds, compositions, and methods. The various embodiments described are meant to provide a variety illustrative examples and should not be construed as descriptions of alternative species. Rather it should be noted that the descriptions of various embodiments provided herein may be of overlapping scope. The embodiments discussed herein are merely illustrative and are not meant to limit the scope of the present invention.

### DEFINITIONS

Unless specifically defined otherwise, the terms used herein are defined below.

“Alkyl” refers to monovalent saturated aliphatic hydrocarbyl groups having from 1 to 10 carbon atoms and preferably 1 to 6 carbon atoms. “C_{x-y}alkyl” refers to alkyl groups having from x to y carbons. This term includes, by way of example, linear and branched hydrocarbyl groups such as methyl (CH₃—), ethyl (CH₃CH₂—), n-propyl (CH₃CH₂CH₂—), isopropyl ((CH₃)₂CH—), n-butyl (CH₃CH₂CH₂CH₂—), isobutyl ((CH₃)₂CHCH₂—), sec-butyl ((CH₃)(CH₃CH₂)CH—), t-butyl ((CH₃)₃C—), n-pentyl (CH₃CH₂CH₂CH₂CH₂—), and neopentyl ((CH₃)₃CCH₂—).

“Substituted alkyl” refers to an alkyl group having from 1 to 5, preferably 1 to 3, or more preferably 1 to 2 substituents selected from the group consisting of alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, azido, carboxyl, carboxyl ester, (carboxyl ester)amino, (carboxyl ester)oxy, cyano, cyanate, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxy, hydroxyamino, alkoxyamino, hydrazino, substituted hydrazino, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, spirocycloalkylidene, SO₃H, substituted sulfonyl, sulfonyloxy, thioacyl, thiocyanate, thiol, alkylthio, and substituted alkylthio, wherein said substituents are defined herein.

“Alkylidene” or “alkylene” refers to divalent saturated aliphatic hydrocarbyl groups having from 1 to 10 carbon atoms and preferably 1 to 6 carbon atoms. “C_{x-y}alkylene” refers to alkylene groups having from x to y carbons. The alkylidene and alkylene groups include branched and straight chain hydrocarbyl groups.

“Substituted alkylidene” or “substituted alkylene” refers to an alkylidene group having from 1 to 5, preferably 1 to 3, or more preferably 1 to 2 substituents selected from the group consisting of alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, azido, carboxyl, carboxyl ester, (carboxyl ester)amino, (carboxyl ester)oxy, cyano, cyanate, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxy, hydroxyamino, alkoxyamino, hydrazino, substituted hydrazino, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, oxo, thione, spirocycloalkylidene, SO₃H, substituted sulfonyl, sulfonyloxy, thioacyl, thiocyanate, thiol, alkylthio, and substituted alkylthio, wherein said substituents are defined herein.

“Alkoxy” refers to the group —O-alkyl wherein alkyl is defined herein. Alkoxy includes, by way of example, methoxy, ethoxy, n-propoxy, isopropoxy, n-butoxy, t-butoxy, sec-butoxy, and n-pentoxy.

“Substituted alkoxy” refers to the group —O-(substituted alkyl) wherein substituted alkyl is defined herein.

“Acyl” refers to the groups H-C(O)—, alkyl-C(O)—, substituted alkyl-C(O)—, alkenyl-C(O)—, substituted alkenyl-C(O)—, alkynyl-C(O)—, substituted alkynyl-C(O)—, cycloalkyl-C(O)—, substituted cycloalkyl-C(O)—, cycloalkenyl-C(O)—, substituted cycloalkenyl-C(O)—, aryl-C(O)—, substituted aryl-C(O)—, substituted hydrazino-C(O)—, heteroaryl-C(O)—, substituted heteroaryl-C(O)—, heterocyclic-C(O)—, and substituted heterocyclic-C(O)—, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, substituted hydrazino, heteroaryl, substituted heteroaryl, heterocyclic and substituted heterocyclic are as defined herein. Acyl includes the “acetyl” group CH₃C(O)—.

“Acylamino” refers to the groups —NR²⁰C(O)alkyl, —NR²⁰C(O)substituted alkyl, —NR²⁰C(O)cycloalkyl, —NR²⁰C(O)substituted cycloalkyl, —NR²⁰C(O)cycloalkenyl, —NR²⁰C(O)substituted cycloalkenyl, —NR²⁰C(O)alkenyl, —NR²⁰C(O)substituted alkenyl, —NR²⁰C(O)alkynyl, —NR²⁰C(O)substituted alkynyl, —NR²⁰C(O)aryl, —NR²⁰C(O)substituted aryl, —NR²⁰C(O)heteroaryl, —NR²⁰C(O)substituted heteroaryl, —NR²⁰C(O)heterocyclic, and —NR²⁰C(O)substituted heterocyclic wherein R²⁰ is hydrogen or alkyl and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic and substituted heterocyclic are as defined herein.

“Acyloxy” refers to the groups alkyl-C(O)O—, substituted alkyl-C(O)O—, alkenyl-C(O)O—, substituted alkenyl-C(O)O—, alkynyl-C(O)O—, substituted alkynyl-C(O)O—, aryl-C(O)O—, substituted aryl-C(O)O—, cycloalkyl-C(O)O—, substituted cycloalkyl-C(O)O—, cycloalkenyl-C(O)O—, substituted cycloalkenyl-C(O)O—, heteroaryl-C(O)O—, substituted heteroaryl-C(O)O—, heterocyclic-C(O)O—, and substituted heterocyclic-C(O)O— wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclyl, and substituted heterocyclyl are as defined herein.

“Amino” refers to the group —NH₂.

“Substituted amino” refers to the group —NR²¹R²² where R²¹ and R²² are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, substituted heterocyclic, —SO₂-alkyl, —SO₂-substituted alkyl, —SO₂-alkenyl, —SO₂-substituted alkenyl, —SO₂-cycloalkyl, —SO₂-substituted cylcoalkyl, —SO₂-cycloalkenyl, —SO₂-substituted cylcoalkenyl, —SO₂-aryl, —SO₂-substituted aryl, —SO₂-heteroaryl, —SO₂-substituted heteroaryl, —SO₂-heterocyclic, and —SO₂-substituted heterocyclic and wherein R²¹ and R²² are optionally joined, together with the nitrogen bound thereto to form a heterocyclic or substituted heterocyclic group, provided that R²¹ and R²² are both not hydrogen, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclyl, and substituted heterocyclyl are as defined herein. When R²¹ is hydrogen and R²² is alkyl, the substituted amino group is sometimes referred to herein as alkylamino. When R²¹ and R²² are alkyl, the substituted amino group is sometimes referred to herein as dialkylamino. When referring to a monosubstituted amino, it is meant that either R²¹ or R²² is hydrogen but not both. When referring to a disubstituted amino, it is meant that neither R²¹ nor R²² are hydrogen.

“Hydroxyamino” refers to the group —NHOH.

“Alkoxyamino” refers to the group —NHO-alkyl wherein alkyl is defined herein.

“Aminocarbonyl” refers to the group —C(O)NR²³R²⁴ where R²³ and R²⁴ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclyl, substituted heterocyclyl, hydroxy, alkoxy, substituted alkoxy, amino, substituted amino, and acylamino, and where R²³ and R²⁴ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic and substituted heterocyclic are as defined herein.

“Aminothiocarbonyl” refers to the group —C(S)NR²³R²⁴ where R²³ and R²⁴ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclyl, and substituted heterocyclyl and where R²³ and R²⁴ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic and substituted heterocyclic are as defined herein.

“Aminocarbonylamino” refers to the group —NR²⁰C(S)NR²³R²⁴ where R²⁰ is hydrogen or alkyl and R²³ and R²⁴ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclyl, and substituted heterocyclyl and where R²³ and R²⁴ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic and substituted heterocyclic are as defined herein.

“Aminothiocarbonylamino” refers to the group —NR²⁰C(S)NR²³R²⁴ where R²⁰ is hydrogen or alkyl and R²³ and R²⁴ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclyl, and substituted heterocyclyl and where R²³ and R²⁴ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic and substituted heterocyclic are as defined herein.

“Aminocarbonyloxy” refers to the group —O—C(O)NR²³R²⁴ where R²³ and R²⁴ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclyl, and substituted heterocyclyl and where R²³ and R²⁴ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic and substituted heterocyclic are as defined herein.

“Aminosulfonyl” refers to the group —SO₂NR²³R²⁴ where R²³ and R²⁴ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclyl, and substituted heterocyclyl and where R²³ and R²⁴ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic and substituted heterocyclic are as defined herein.

“Aminosulfonyloxy” refers to the group —O—SO₂NR²³R²⁴ where R²³ and R²⁴ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclyl, and substituted heterocyclyl and where R²³ and R²⁴ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic and substituted heterocyclic are as defined herein.

“Aminosulfonylamino” refers to the group —NR²⁰—SO₂NR²³R²⁴ where R²⁰ is hydrogen or alkyl and R²³ and R²⁴ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclyl, and substituted heterocyclyl and where R²³ and R²⁴ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkyenyl, aryl, substituted aryl, heteroaryl, substituted heteroaryl, heterocyclic and substituted heterocyclic are as defined herein.

“Amidino” refers to the group —C(═NR²⁵)NR²³R²⁴ where R²⁵, R²³, and R²⁴ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclyl, and substituted heterocyclyl and where R²³ and R²⁴ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic and substituted heterocyclic are as defined herein.

“Aryl” or “Ar” refers to a monovalent aromatic carbocyclic group of from 6 to 14 carbon atoms having a single ring (e.g., phenyl) or multiple condensed rings (e.g., naphthyl or anthryl) which condensed rings may or may not be aromatic (e.g., 2-benzoxazolinone, 2H-1,4-benzoxazin-3(4H)-one-7-yl, and the like) provided that the point of attachment is at an aromatic carbon atom. Aryl groups include phenyl and naphthyl.

“Substituted aryl” refers to aryl groups which are substituted with 1 to 5, preferably 1 to 3, or more preferably 1 to 2 substituents selected from the group consisting of alkyl, substituted alkyl, alkenyl, substituted alkenyl, alkynyl, substituted alkynyl, alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, azido, carboxyl, carboxyl ester, (carboxyl ester)amino, (carboxyl ester)oxy, cyano, cyanate, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxy, hydroxyamino, alkoxyamino, hydrazino, substituted hydrazino, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, sulfonyloxy, thioacyl, thiocyanate, thiol, alkylthio, and substituted alkylthio, wherein said substituents are defined herein.

“Aryloxy” refers to the group —O-aryl, where aryl is as defined herein, that includes, by way of example, phenoxy and naphthoxy.

“Substituted aryloxy” refers to the group —O-(substituted aryl) where substituted aryl is as defined herein.

“Arylthio” refers to the group —S-aryl, where aryl is as defined herein.

“Substituted arylthio” refers to the group —S-(substituted aryl), where substituted aryl is as defined herein.

“Alkenyl” refers to alkenyl groups having from 2 to 6 carbon atoms and preferably 2 to 4 carbon atoms and having at least 1 and preferably from 1 to 2 sites of vinyl unsaturation (>C═C<). Such groups are exemplified, for example, by vinyl, allyl, and but-3-en-yl.

“Substituted alkenyl” refers to alkenyl groups having from 1 to 3 substituents, and preferably 1 to 2 substituents, selected from the group consisting of alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, sulfonyloxy, thioacyl, thiol, alkylthio, and substituted alkylthio, wherein said substituents are defined herein and with the proviso that any hydroxy or thiol substitution is not attached to a vinyl (unsaturated) carbon atom.

“Alkynyl” refers to hydrocarbyl groups having from 2 to 6 carbon atoms and preferably 2 to 3 carbon atoms and having at least 1 and preferably from 1 to 2 sites of acetylenic unsaturation (—CC—).

“Substituted alkynyl” refers to alkynyl groups having from 1 to 3 substituents, and preferably 1 to 2 substituents, selected from the group consisting of alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, sulfonyloxy, thioacyl, thiol, alkylthio, and substituted alkylthio, wherein said substituents are defined herein and with the proviso that any hydroxy or thiol substitution is not attached to an acetylenic carbon atom.

“Azido” refers to the group —N₃.

“Hydrazino” refers to the group —NHNH₂.

“Substituted hydrazino” refers to the group —NR²⁶NR²⁷R²⁸ where R²⁶, R²⁷, and R²⁸ are independently selected from the group consisting of hydrogen, alkyl, substituted alkyl, alkenyl, substituted alkenyl, alkynyl, substituted alkynyl, aryl, substituted aryl, carboxyl ester, cycloalkyl, substituted cycloalkyl, cycloalkenyl, substituted cycloalkenyl, heteroaryl, substituted heteroaryl, heterocyclic, substituted heterocyclic, —SO₂-alkyl, —SO₂-substituted alkyl, —SO₂-alkenyl, —SO₂-substituted alkenyl, —SO₂-cycloalkyl, —SO₂-substituted cylcoalkyl, —SO₂-cycloalkenyl, —SO₂-substituted cycloalkenyl, —SO₂-aryl, —SO₂-substituted aryl, —SO₂-heteroaryl, —SO₂-substituted heteroaryl, —SO₂-heterocyclic, and —SO₂-substituted heterocyclic and wherein R²⁷ and R²⁸ are optionally joined, together with the nitrogen bound thereto to form a heterocyclic or substituted heterocyclic group, provided that R²⁷ and R²⁸ are both not hydrogen, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclyl, and substituted heterocyclyl are as defined herein.

“Cyano” or “carbonitrile” refers to the group —CN.

“Cyanate” refers to the group —OCN.

“Carbonyl” refers to the divalent group —C(O)— which is equivalent to —C(═O)—.

“Carboxyl” or “carboxy” refers to —COOH or salts thereof.

“Carboxyl ester” or “carboxy ester” refers to the groups —C(O)O-alkyl, —C(O)O-substituted alkyl, —C(O)O-alkenyl, —C(O)O-substituted alkenyl, —C(O)O-alkynyl, —C(O)O-substituted alkynyl, —C(O)O-aryl, —C(O)O-substituted aryl, —C(O)β-cycloalkyl, —C(O)O-substituted cycloalkyl, —C(O)β-cycloalkenyl, —C(O)O-substituted cycloalkenyl, —C(O)O-heteroaryl, —C(O)O-substituted heteroaryl, —C(O)O-heterocyclic, and —C(O)O-substituted heterocyclic wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclyl, and substituted heterocyclyl are as defined herein.

“(Carboxyl ester)amino” refers to the group —NR²⁰—C(O)O-alkyl, —NR²⁰—C(O)O-substituted alkyl, —NR²⁰—C(O)O-alkenyl, —NR²⁰—C(O)O-substituted alkenyl, —NR²⁰—C(O)O-alkynyl, —NR²⁰—C(O)O-substituted alkynyl, —NR²⁰—C(O)O-aryl, —NR²⁰—C(O)O-substituted aryl, —NR²⁰—C(O)β-cycloalkyl, —NR²⁰—C(O)O-substituted cycloalkyl, —NR²⁰—C(O)β-cycloalkenyl, —NR²⁰—C(O)O-substituted cycloalkenyl, —NR²⁰—C(O)O-heteroaryl, —NR²⁰—C(O)O-substituted heteroaryl, —NR²⁰—C(O)O-heterocyclic, and —NR²⁰—C(O)O-substituted heterocyclic wherein R²⁰ is alkyl or hydrogen, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclyl, and substituted heterocyclyl are as defined herein.

“(Carboxyl ester)oxy” refers to the group —O—C(O)O-alkyl, —O—C(O)O-substituted alkyl, —O—C(O)O-alkenyl, —O—C(O)O-substituted alkenyl, —O—C(O)O-alkynyl, —O—C(O)O-substituted alkynyl, —O—C(O)O-aryl, —O—C(O)O-substituted aryl, —O—C(O)β-cycloalkyl, —O—C(O)O-substituted cycloalkyl, —O—C(O)β-cycloalkenyl, —O—C(O)O-substituted cycloalkenyl, —O—C(O)O-heteroaryl, —O—C(O)O-substituted heteroaryl, —O—C(O)O-heterocyclic, and —O—C(O)O-substituted heterocyclic wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclyl, and substituted heterocyclyl are as defined herein.

“Cycloalkyl” refers to cyclic alkyl groups of from 3 to 10 carbon atoms having single or multiple cyclic rings including fused, bridged, and spiro ing systems. In fused ring systems, one or more the rings can be cycloalkyl, heterocyclic, aryl, or heteroaryl provided that the point of attachment is through the cycloalkyl ring. Examples of suitable cycloalkyl groups include, for instance, adamantyl, cyclopropyl, cyclobutyl, cyclopentyl, and cyclooctyl. “C_{x-y}cycloalkyl” refers to cycloalkyl groups having x to y carbons.

“Cycloalkenyl” refers to non-aromatic cyclic alkyl groups of from 4 to 10 carbon atoms having single or multiple cyclic rings and having at least one >C═C<ring unsaturation and preferably from 1 to 2 sites of >C═C<ring unsaturation. “C_{x-y}cycloalkenyl” refers to cycloalkenyl groups having x to y carbons.

“Substituted cycloalkyl” and “substituted cycloalkenyl” refers to a cycloalkyl or cycloalkenyl group having from 1 to 5 or preferably 1 to 3 substituents selected from the group consisting of oxo, thione, alkyl, substituted alkyl, alkenyl, substituted alkenyl, alkynyl, substituted alkynyl, alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, azido, carboxyl, carboxyl ester, (carboxyl ester)amino, (carboxyl ester)oxy, cyano, cyanate, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxy, hydroxyamino, alkoxyamino, hydrazino, substituted hydrazino, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, sulfonyloxy, thioacyl, thiocyanate, thiol, alkylthio, and substituted alkylthio, wherein said substituents are defined herein.

“Cycloalkyloxy” refers to —O-cycloalkyl.

“Substituted cycloalkyloxy refers to —O-(substituted cycloalkyl).

“Cycloalkylthio” refers to —S-cycloalkyl.

“Substituted cycloalkylthio” refers to —S-(substituted cycloalkyl).

“Cycloalkenyloxy” refers to —O-cycloalkenyl.

“Substituted cycloalkenyloxy” refers to —O-(substituted cycloalkenyl).

“Cycloalkenylthio” refers to —S-cycloalkenyl.

“Substituted cycloalkenylthio” refers to —S-(substituted cycloalkenyl).

“Guanidino” refers to the group —NHC(═NH)NH₂.

“Substituted guanidino” refers to —NR²⁹C(═NR²⁹)N(R²⁹)₂ where each R²⁹ is independently selected from the group consisting of hydrogen, alkyl, substituted alkyl, aryl, substituted aryl, heteroaryl, substituted heteroaryl, heterocyclyl, and substituted heterocyclyl and two R²⁹ groups attached to a common guanidino nitrogen atom are optionally joined together with the nitrogen bound thereto to form a heterocyclic or substituted heterocyclic group, provided that at least one R²⁹ is not hydrogen, and wherein said substituents are as defined herein.

“Halo” or “halogen” refers to fluoro, chloro, bromo and iodo.

“Haloalkyl” refers to substitution of alkyl groups with 1 to 5 or preferably 1 to 3 halo groups.

“Haloalkoxy” refers to substitution of alkoxy groups with 1 to 5 or preferably 1 to 3 halo groups.

“Hydroxy” or “hydroxyl” refers to the group —OH.

“Heteroaryl” refers to an aromatic group of from 1 to 10 carbon atoms and 1 to 4 heteroatoms selected from the group consisting of oxygen, nitrogen and sulfur within the ring. Such heteroaryl groups can have a single ring (e.g., pyridinyl or furyl) or multiple condensed rings (e.g., indolizinyl or benzothienyl) wherein the condensed rings may or may not be aromatic and/or contain a heteroatom provided that the point of attachment is through an atom of the aromatic heteroaryl group. In one embodiment, the nitrogen and/or the sulfur ring atom(s) of the heteroaryl group are optionally oxidized to provide for the N-oxide (N→O), sulfinyl, or sulfonyl moieties. Heteroaryls include pyridinyl, pyrrolyl, indolyl, thiophenyl, and furanyl.

“Substituted heteroaryl” refers to heteroaryl groups that are substituted with from 1 to 5, preferably 1 to 3, or more preferably 1 to 2 substituents selected from the group consisting of the same group of substituents defined for substituted aryl.

“Heteroaryloxy” refers to —O-heteroaryl.

“Substituted heteroaryloxy refers to the group —O-(substituted heteroaryl).

“Heteroarylthio” refers to the group —S-heteroaryl.

“Substituted heteroarylthio” refers to the group —S-(substituted heteroaryl).

“Heterocycle” or “heterocyclic” or “heterocycloalkyl” or “heterocyclyl” refers to a saturated, partially saturated, or unsaturated group (but not aromatic) having a single ring or multiple condensed rings, including fused bridged and spirocycyl ring systems, from 1 to 10 carbon atoms and from 1 to 4 hetero atoms selected from the group consisting of nitrogen, sulfur or oxygen within the ring wherein, in fused ring systems, one or more the rings can be cycloalkyl, aryl or heteroaryl provided that the point of attachment is through the non-aromatic ring. In one embodiment, the nitrogen and/or sulfur atom(s) of the heterocyclic group are optionally oxidized to provide for the N-oxide, sulfinyl, sulfonyl moieties.

“Substituted heterocyclic” or “substituted heterocycloalkyl” or “substituted heterocyclyl” refers to heterocyclyl groups that are substituted with from 1 to 5 or preferably 1 to 3 of the same substituents as defined for substituted cycloalkyl.

“Heterocyclyloxy” refers to the group —O-heterocycyl.

“Substituted heterocyclyloxy” refers to the group —O-(substituted heterocycyl).

“Heterocyclylthio” refers to the group —S-heterocycyl.

“Substituted heterocyclylthio” refers to the group —S-(substituted heterocycyl).

Examples of heterocycle and heteroaryls include, but are not limited to, azetidine, pyrrole, imidazole, pyrazole, pyridine, pyrazine, pyrimidine, pyridazine, indolizine, isoindole, indole, dihydroindole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, indoline, phthalimide, 1,2,3,4-tetrahydroisoquinoline, 4,5,6,7-tetrahydrobenzo[b]thiophene, thiazole, thiazolidine, thiophene, benzo[b]thiophene, morpholinyl, thiomorpholinyl (also referred to as thiamorpholinyl), 1,1-dioxothiomorpholinyl, piperidinyl, pyrrolidine, and tetrahydrofuranyl.

“Nitro” refers to the group —NO₂.

“Oxo” refers to the atom (═O).

“Oxide” refers to products resulting from the oxidation of one or more heteroatoms. Examples include N-oxides, sulfoxides, and sulfones.

“Spirocyclyl” refers to divalent cyclic groups from 3 to 10 carbon atoms having a cycloalkyl or heterocyclyl ring with a spiro union (the union formed by a single atom which is the only common member of the rings) as exemplified by the following structure:

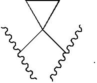

“Spirocycloalkyl” or “spirocycloalkylidene” refers to divalent cyclic groups having a cycloalkyl ring with a spiro union, as described for spirocyclyl.

“Sulfonyl” refers to the divalent group —S(O)₂—.

“Substituted sulfonyl” refers to the group —SO₂-alkyl, —SO₂-substituted alkyl, —SO₂-alkenyl, —SO₂-substituted alkenyl, —SO₂-cycloalkyl, —SO₂-substituted cylcoalkyl, —SO₂-cycloalkenyl, —SO₂-substituted cylcoalkenyl, —SO₂-aryl, —SO₂-substituted aryl, —SO₂-heteroaryl, —SO₂-substituted heteroaryl, —SO₂-heterocyclic, —SO₂-substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic and substituted heterocyclic are as defined herein. Substituted sulfonyl includes groups such as methyl-SO₂—, phenyl-SO₂—, and 4-methylphenyl-SO₂—.

“Sulfonyloxy” refers to the group —OSO₂-alkyl, —OSO₂-substituted alkyl, —OSO₂-alkenyl, —OSO₂-substituted alkenyl, —OSO₂-cycloalkyl, —OSO₂-substituted cylcoalkyl, —OSO₂-cycloalkenyl, —OSO₂-substituted cylcoalkenyl, —OSO₂-aryl, —OSO₂-substituted aryl, —OSO₂-heteroaryl, —OSO₂-substituted heteroaryl, —OSO₂-heterocyclic, —OSO₂-substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic and substituted heterocyclic are as defined herein.

“Thioacyl” refers to the groups H-C(S)—, alkyl-C(S)—, substituted alkyl-C(S)—, alkenyl-C(S)—, substituted alkenyl-C(S)—, alkynyl-C(S)—, substituted alkynyl-C(S)—, cycloalkyl-C(S)—, substituted cycloalkyl-C(S)—, cycloalkenyl-C(S)—, substituted cycloalkenyl-C(S)—, aryl-C(S)—, substituted aryl-C(S)—, heteroaryl-C(S)—, substituted heteroaryl-C(S)—, heterocyclic-C(S)—, and substituted heterocyclic-C(S)—, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic and substituted heterocyclic are as defined herein.

“Thiol” refers to the group —SH.

“Alkylthio” refers to the group —S-alkyl wherein alkyl is as defined herein.

“Substituted alkylthio” refers to the group —S-(substituted alkyl) wherein substituted alkyl is as defined herein.

“Thiocarbonyl” refers to the divalent group —C(S)— which is equivalent to —C(═S)—.

“Thione” refers to the atom (═S).

“Thiocyanate” refers to the group —SCN.

“Compound” and “compounds” as used herein refers to a compound encompassed by the generic formulae disclosed herein, any subgenus of those generic formulae, and any specific compounds within the generic and subgeneric formulae, including the oxide, ester, prodrug, pharmaceutically acceptable salt, or solvate thereof. The term further includes the stereoisomers and tautomers of the compound or compounds.

“Solvate” or “solvates” of a compound refer to those compounds, where compounds is as defined above, that are bound to a stoichiometric or non-stoichiometric amount of a solvent. Solvates includes solvates of the oxide, ester, prodrug, or pharmaceutically acceptable salt of the disclosed generic and subgeneric formulae. Preferred solvents are volatile, non-toxic, and/or acceptable for administration to humans in trace amounts. Suitable solvates include water.

“Stereoisomer” or “stereoisomers” refer to compounds that differ in the chirality of one or more stereocenters. Stereoisomers include enantiomers and diastereomers.

“Tautomer” refer to alternate forms of a compound that differ in the position of a proton, such as enol-keto and imine-enamine tautomers, or the tautomeric forms of heteroaryl groups containing a ring atom attached to both a ring —NH— moiety and a ring ═N— moeity such as pyrazoles, imidazoles, benzimidazoles, triazoles, and tetrazoles.

“Prodrug” refers to any derivative of a compound of the embodiments that is capable of directly or indirectly providing a compound of the embodiments or an active metabolite or residue thereof when administered to a subject. Particularly favored derivatives and prodrugs are those that increase the bioavailability of the compounds of the embodiments when such compounds are administered to a subject (e.g., by allowing an orally administered compound to be more readily absorbed into the blood) or which enhance delivery of the parent compound to a biological compartment (e.g., the brain or lymphatic system) relative to the parent species. Prodrugs include ester forms of the compounds of the invention. Examples of ester prodrugs include formate, acetate, propionate, butyrate, acrylate, and ethylsuccinate derivatives. An general overview of prodrugs is provided in T. Higuchi and V. Stella, Pro-drugs as Novel Delivery Systems, Vol. 14 of the A.C.S. Symposium Series, and in Edward B. Roche, ed., Bioreversible Carriers in Drug Design, American Pharmaceutical Association and Pergamon Press, 1987, both of which are incorporated herein by reference.

“Pharmaceutically acceptable salt” refers to pharmaceutically acceptable salts derived from a variety of organic and inorganic counter ions well known in the art and include, by way of example only, sodium, potassium, calcium, magnesium, ammonium, and tetraalkylammonium, and when the molecule contains a basic functionality, salts of organic or inorganic acids, such as hydrochloride, hydrobromide, tartrate, mesylate, acetate, maleate, and oxalate. Pharmaceutically acceptable salt of a compound refers to pharmaceutically acceptable salts including salts of the oxide, ester, or prodrug of the disclosed generic and subgeneric formulae.

“Patient” refers to mammals and includes humans and non-human mammals.

“Treating” or “treatment” of a disease in a patient refers to 1) preventing the disease from occurring in a patient that is predisposed or does not yet display symptoms of the disease; 2) inhibiting the disease or arresting its development; or 3) ameliorating or causing regression of the disease.

Reference to “selective” inhibition, refers to a compound, composition, or chemotype that preferentially inhibits a particular target or class of targets. Reference to “selective inhibition of CSF-1R″ indicates the preferential inhibition of CSF-1R and optionally like kinase receptors such as PDGFR. In some embodiments, selective inhibition of CSF-1R refers to preferential inhibition of CSF-1R over Raf kinase. “Selective,” “targeted,” “specific,” or “preferential” inhibition is not intended to mean complete absence of inhibitory activity with respect to all other kinases or receptors.

“CSF-1R inhibitor” refers to a compound that can inhibit CSF-1R. Preferably, a CSF-1R inhibitor is selective of CSF-1R over other targets. In an embodiment, a CSF-1R inhibitor has selective inhibition of CSF-1R over Raf kinase. In another embodiment, such selective inhibition refers to at least a 2:1 binding preference of a compound of this invention to CSF-1R relative to Raf kinase. In still other embodiments the binding preference is at least 5:1. In yet other embodiments the binding preference is at least 10:1.

Unless indicated otherwise, the nomenclature of substituents that are not explicitly defined herein are arrived at by naming the terminal portion of the functionality followed by the adjacent functionality toward the point of attachment. For example, the substituent “arylalkyloxycabonyl” refers to the group (aryl)-(alkyl)-O—C(O)—.

It is understood that in all substituted groups defined above, polymers arrived at by defining substituents with further substituents to themselves (e.g., substituted aryl having a substituted aryl group as a substituent which is itself substituted with a substituted aryl group, which is further substituted by a substituted aryl group etc.) are not intended for inclusion herein. In such cases, the maximum number of such substitutions is three. For example, serial substitutions of substituted aryl groups with two other substituted aryl groups are limited to -substituted aryl-(substituted aryl)-substituted aryl.

Similarly, it is understood that the above definitions are not intended to include impermissible substitution patterns (e.g., methyl substituted with 5 fluoro groups). Such impermissible substitution patterns are well known to the skilled artisan.

In some embodiments of its compound aspect, the invention provides a compound of Formula (II):

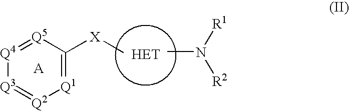

or an oxide, ester, prodrug, pharmaceutically acceptable salt, or solvate thereof, wherein:

A is a six-member ring where each of Q¹, Q², Q³, Q⁴ and Q⁵ is independently C—R³ or N, provided that at least one of Q¹, Q², Q³, Q⁴ and Q⁵ is N and at most three of Q¹, Q², Q³, Q⁴ and Q⁵ are N;

each R³ is independently hydrogen or R^{3a}, where R^{3a} is selected from the group consisting of halo, alkyl, substituted alkyl, alkenyl, substituted alkenyl, alkynyl, substituted alkynyl, carbonitrile, aryl, substituted aryl, cycloalkyl, substituted cycloalkyl, cycloalkenyl, substituted cycloalkenyl, heteroaryl, substituted heteroaryl, heterocyclic, substituted heterocyclic, amino, substituted amino, acylamino, alkoxy, substituted alkoxy, carboxyl, carboxyl ester, substituted sulfonyl, aminosulfonyl, and aminocarbonyl; or two adjacent R^{3a} groups together form a aryl, substituted aryl, heterocyclic, substituted heterocyclic, heteroaryl, or substituted heteroaryl group that is fused to ring A;

HET is a bicyclic ring selected from the group consisting of:
(a) a [6,6] fused bicyclic ring selected from the group consisting of:

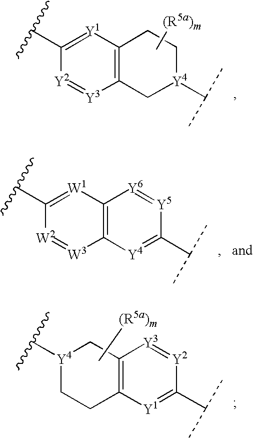

(b) a [5,6] fused bicyclic ring selected from the group consisting of:

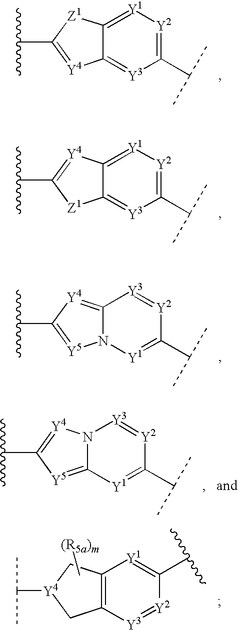

(c) a [6,5] fused bicyclic ring selected from the group consisting of:

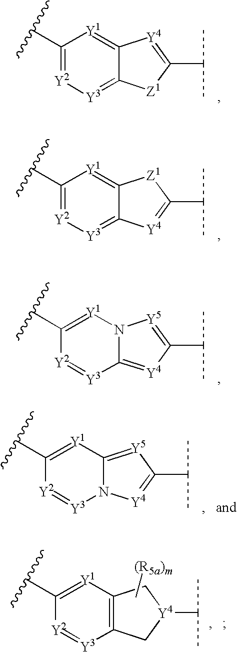

and
(d) a [5,5] fused bicyclic ring selected from the group consisting of:

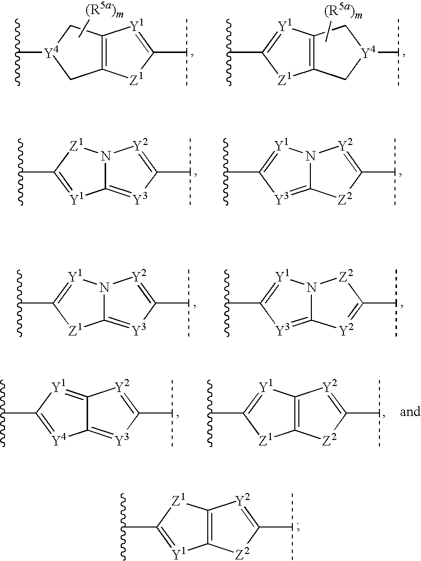

wherein the wavy line represents point of connection with X and dashed line represents point of connection with —NR¹R²;

R¹ and R² are independently selected from the group consisting of hydrogen, alkyl, substituted alkyl, cycloalkyl, substituted cycloalkyl, cycloalkenyl, substituted cycloalkenyl, aryl, substituted aryl, heterocyclyl, substituted heterocyclyl, heteroaryl, substituted heteroaryl, acyl, and aminocarbonyl, or R¹ and R² are taken together to form a group selected from heterocyclyl, substituted heterocyclyl, heteroaryl, and substituted heteroaryl; provided R¹ and R² are not both hydrogen;

Y¹, Y², Y³, Y⁴, Y⁵, and Y⁶ are independently selected from the group consisting of C—R⁵ and N;

W¹, W², and W³ are independently selected from the group consisting of C—R⁵ and N, provided that at least one of W¹, W², and W³ is N;

each R⁵ is independently hydrogen or R^{5a};

R^{5a} is independently selected from the group consisting of alkyl, substituted alkyl, alkoxy, substituted alkoxy, amino, substituted amino, and halo, or optionally when m is at least 2, two R^{5a} together with the carbon atom to which they are both attached from a C═O or C═S group;

m is 0, 1, 2, 3, 4, or 5;

Z¹ and Z² are independently selected from the group consisting of C(—R⁵)₂, O, N—R⁶, S, and S(O); where each R⁶ is independently selected from the group consisting of hydrogen, alkyl, and substituted alkyl; and

X is selected from the group consisting of O, S, S(O), S(O)₂, and N—R⁴, wherein R⁴ is hydrogen, alkyl, or substituted alkyl; provided that when X is O, HET is not

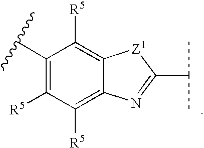

In some embodiments, HET is selected from the group consisting of:

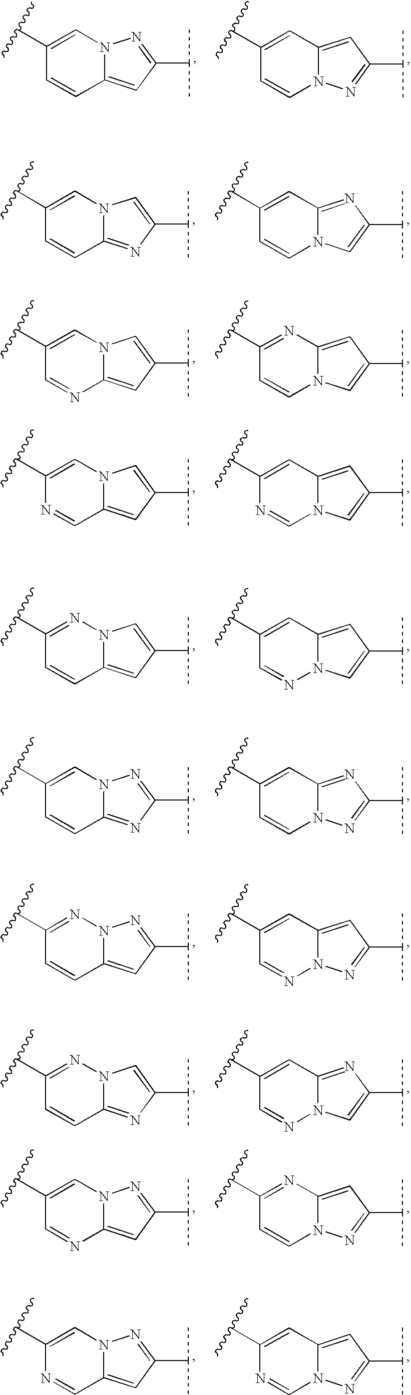

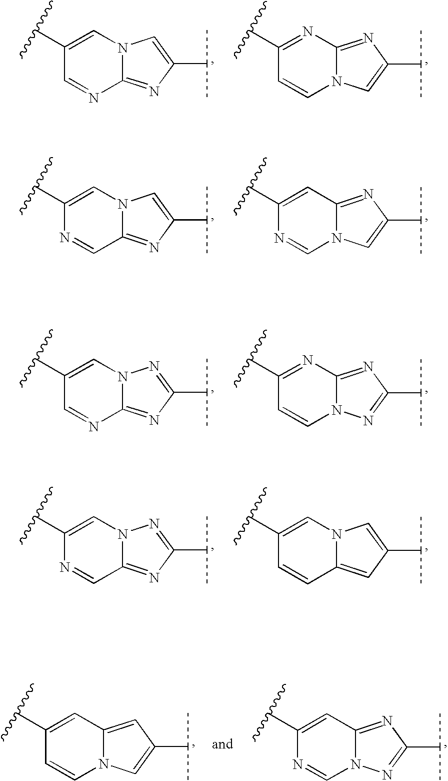

wherein each bicyclic ring is optionally substituted with one to four R^{5a} groups and R^{5a} is as defined herein.

In some embodiments, HET is selected from the group consisting of:

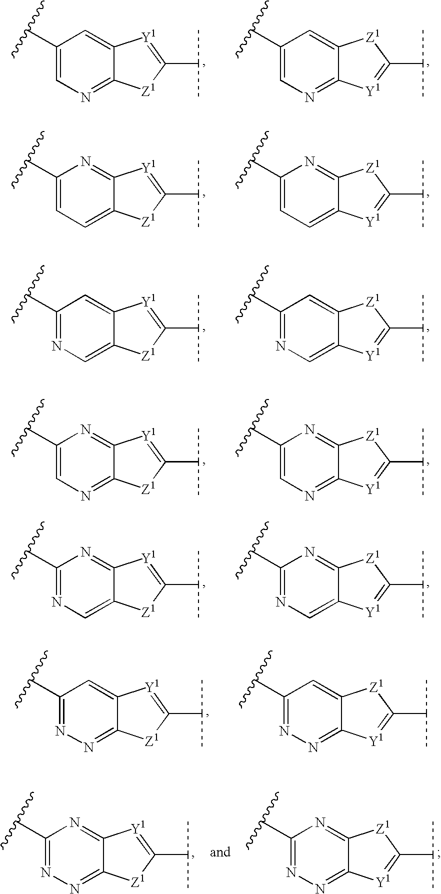

wherein each bicyclic ring is optionally substituted with one to three R^{5a} groups, and wherein R^{5a}, Y¹ and Z¹ are as defined herein.

In some embodiments, HET is selected from the group consisting of:

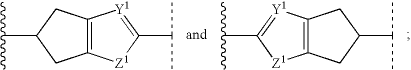

and wherein Y¹ and Z¹ are as defined herein.

In some embodiments, HET is selected from the group consisting of:

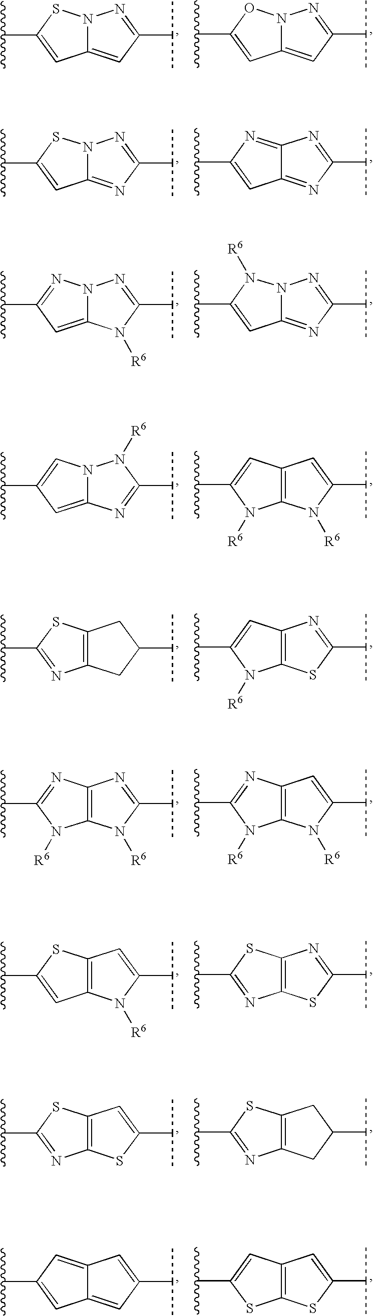

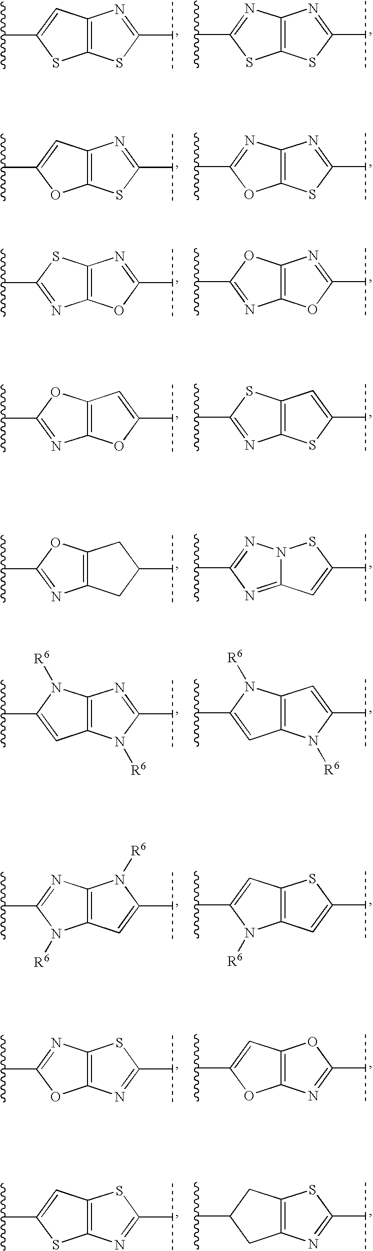

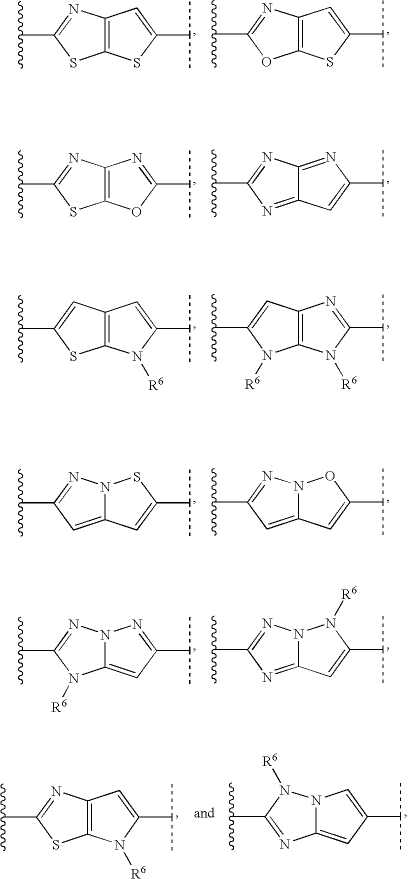

wherein each bicyclic ring is optionally substituted with one to two R^{5a} groups, where R^{5a} and R⁶ are as defined herein.

In some embodiments of its compound aspect, the invention provides a compound of Formula (III):

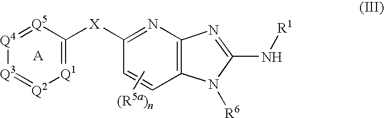

or an oxide, ester, prodrug, pharmaceutically acceptable salt, or solvate thereof,

wherein:

Q¹, Q², Q³, Q⁴, Q⁵, X, R⁶, and R^{5a} are as defined herein; and

n is 0, 1, or 2.

In some embodiments of its compound aspect, the invention provides a compound of Formula (IV):

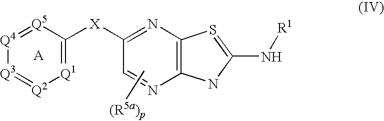

or an oxide, ester, prodrug, pharmaceutically acceptable salt, or solvate thereof,

wherein:

Q¹, Q², Q³, Q⁴, Q⁵, X, and R^{5a} are as defined herein; and

p is 0 or 1.

In some embodiments of its compound aspect, the invention provides a compound of Formula (V):

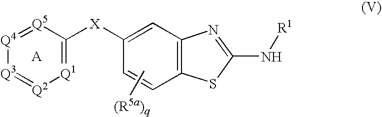

or an oxide, ester, prodrug, pharmaceutically acceptable salt, or solvate thereof,

wherein:

Q¹, Q², Q³, Q⁴, Q⁵, X, R¹, and R^{5a} are as defined herein; and

q is 0, 1, 2 or 3.

In some embodiments of its compound aspect, the invention provides a compound of Formula (VI):

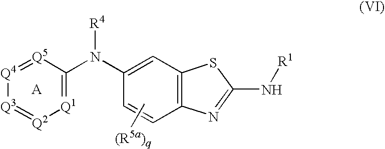

or an oxide, ester, prodrug, pharmaceutically acceptable salt, or solvate thereof,

wherein:

Q¹, Q², Q³, Q⁴, Q⁵, R¹, R⁴, and R^{5a} are as defined herein; and

q is 0, 1, 2 or 3.

Various embodiments relating to a compound of Formula (II)-(VI) or an oxide, ester, prodrug, pharmaceutically acceptable salt, or solvate thereof are given below. These embodiments when referring to different substituents or variables can be combined with each other or with any other embodiments described in this application. In some aspects, provided are compounds of Formula (II)-(VI) having one or more of the following features.

In some embodiments, the compound is a salt.

In some embodiments, X is S.

In some embodiments, X is O.

In some embodiments, X is S(O).

In some embodiments, the oxide is an oxide wherein X is S(O)₂.

In some embodiments, X is N—R⁴.

In some embodiments, R¹ is selected from the group consisting of alkyl, substituted alkyl, cycloalkyl, substituted cycloalkyl, aryl, substituted aryl, heteroaryl, substituted heteroaryl, acyl, and aminocarbonyl.

In some embodiments, R¹ is L-R⁷,

wherein:

L is selected from the group consisting of a covalent bond, alkylene, substituted alkylene, —C(O) and —C(O)—NH—; and

R⁷ is selected from the group consisting of alkyl, substituted alkyl, cycloalkyl, substituted cycloalkyl, cycloalkenyl, substituted cycloalkenyl, aryl, substituted aryl, heterocyclyl, substituted heterocyclic, heteroaryl, and substituted heteroaryl.

In some embodiments, L is a covalent bond.

In some embodiments, L is C₁₋₃alkylene substituted with 0, 1, 2, or 3 substituents independently selected from alkyl, substituted alkyl, hydroxy, alkoxy, haloalkoxy, aminocarbonyl, carboxyl ester, and carboxyl.

In some embodiments, L is selected from the group consisting of —CH₂—, —CH(CH₃)—, and —CH₂—CH₂—.

In some embodiments, L is —C(O)— or —C(O)—NH—.

In some embodiments, R⁷ is an optionally substituted ring selected from phenyl, furan-2-yl, furan-3-yl, tetrahydropyran-2-yl, tetrahydropyran-3-yl, tetrahydropyran-4-yl, cyclopropyl, cyclobutyl, cyclopentyl, cyclohexyl, cycloheptyl, cyclooctyl, cyclohexenyl, pyridin-2-yl, pyridin-3-yl, pyridin-4-yl, 2,3-dihydrobenzofuran, thiazolyl, 2,3-dihydrobenzo[b][1,4]dioxine, 3,4-dihydro-2H-benzo[b][1,4]dioxepine, pyrazinyl, pyrrolidinyl, piperidinyl, piperidinone, pyrrolidinone, pyridin-2(1H)-one, morpholino, napthyl, bicyclo[3.1.1]heptane, bicyclo[2.2.1]heptane, 1,2,3,4-tetrahydronaphthalene, 2,3-dihydro-1H-indene, benzo[d][1,3]dioxolyl, and azepan-2-one.

In some embodiments, R⁷ is selected from the group consisting of:

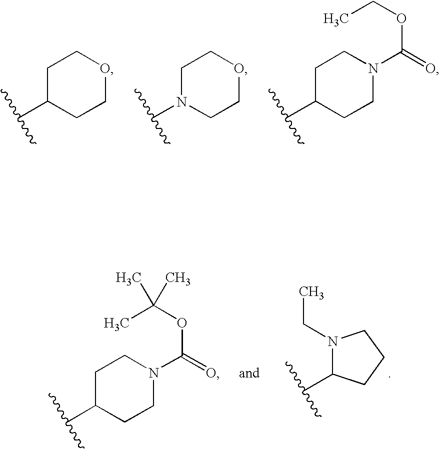

In some embodiments, R⁷ is cycloalkyl or substituted cycloalkyl.

In some embodiments, R⁷ is selected from the group consisting of:

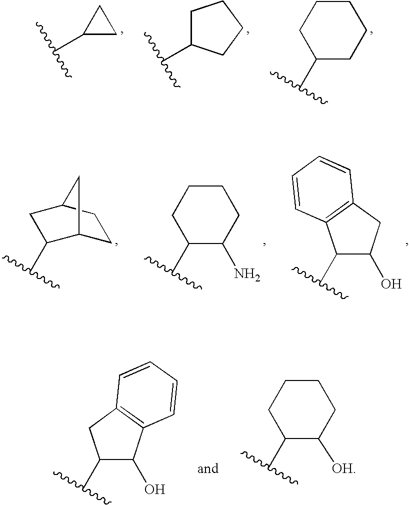

In some embodiments, R⁷ is

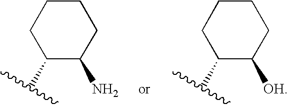

In some embodiments, L-R⁷ is selected from the group consisting of:

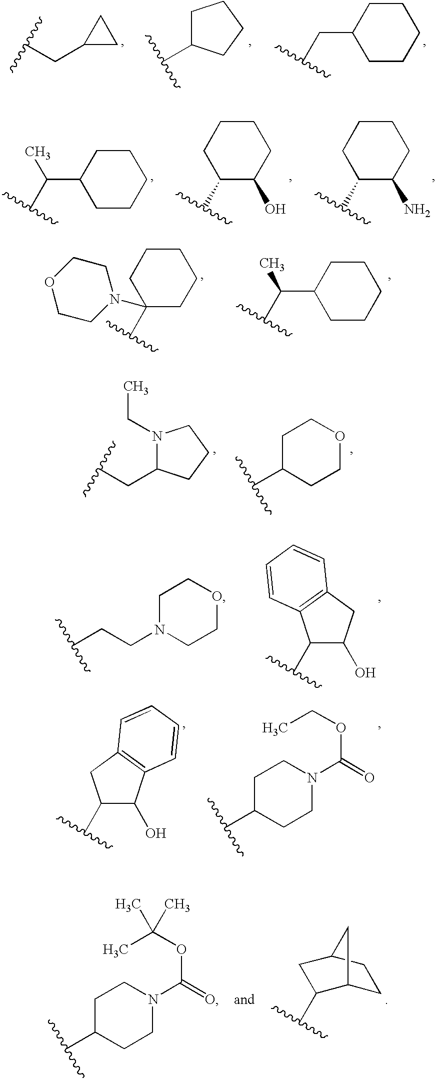

In some embodiments, R⁷ is a ring selected from the group consisting of:

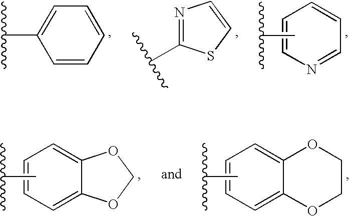

wherein said ring is optionally substituted with one to three substituents independently selected from the group consisting of: halo, alkyl, hydroxy, alkoxy, amino,

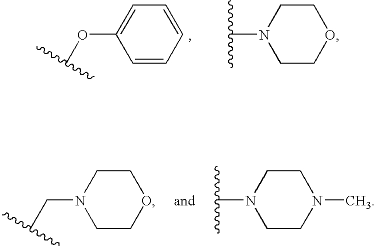

In some embodiments, R⁷ is selected from the group consisting of:

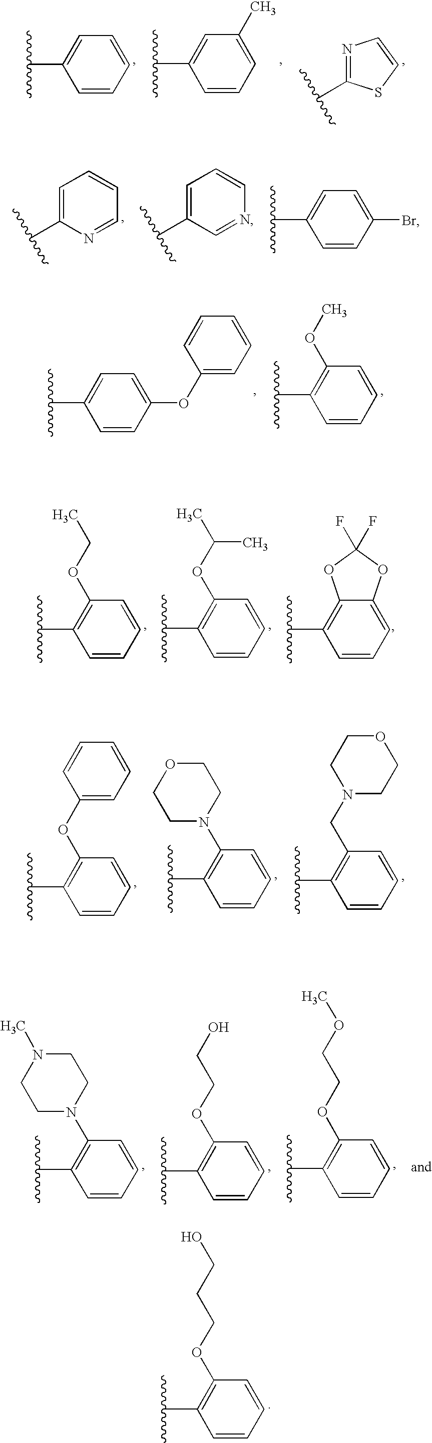

In some embodiments, L-R⁷ is selected from the group consisting of:

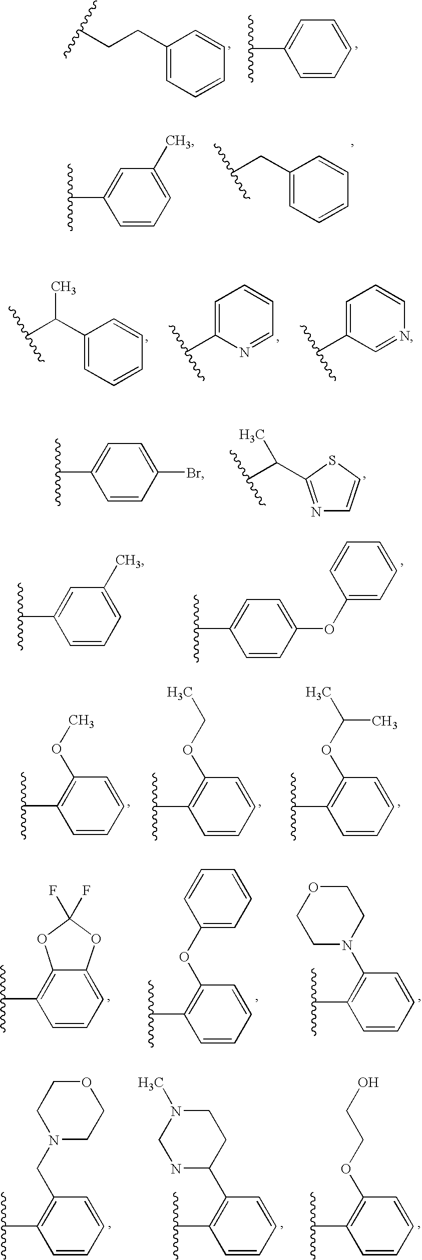

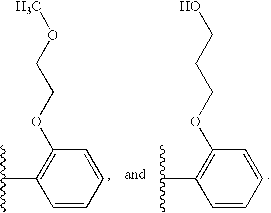

In some embodiments, L-R⁷ is selected from the group consisting of

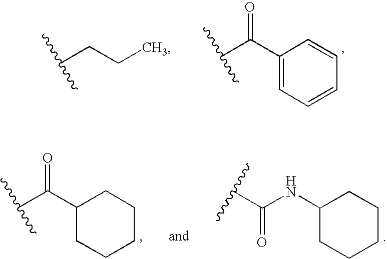

In some embodiments, R² is hydrogen or methyl.

In some embodiments, each R^{3a} is independently selected from the group consisting of halo, nitro, hydroxyamino, alkyl, substituted alkyl, alkenyl, substituted alkenyl, alkynyl, substituted alkynyl, carbonitrile, aryl, substituted aryl, cycloalkyl, substituted cycloalkyl, cycloalkenyl, substituted cycloalkenyl, heteroaryl, substituted heteroaryl, heterocyclic, substituted heterocyclic, amino, substituted amino, acyl, acylamino, alkoxy, substituted alkoxy, carboxyl, carboxyl ester, substituted sulfonyl, aminosulfonyl, and aminocarbonyl.

In some embodiments, each R^{3a} group is selected from the group consisting of F, Cl, Br, —NHOH, —NO₂, —CN, amino, substituted amino, C₁₋₃alkyl, C₃₋₇cycloalkyl, C₃₋₇cycloalkenyl, pyrrolidinyl, piperidinyl, piperidinone, pyrrolidinone, pyridin-2(1H)-one, morpholino, thiamorpholino, phenyl, pyrrolyl, pyrazolyl, imidazolyl, isoxazolyl, isothiazolyl, furyl, thienyl, furanyl, pyridinyl, pyrazinyl, pyrimidinyl, pyridazinyl, napthyl, and pyrrolo[2,3-b]pyridinyl, wherein said C₁₋₃alkyl, C₃₋₇cycloalkyl, C₃₋₇cycloalkenyl, pyrrolidinyl, piperidinyl, piperidinone, pyrrolidinone, pyridin-2(1H)-one, morpholino, thiamorpholino, phenyl, pyrrolyl, pyrazolyl, imidazolyl, isoxazolyl, isothiazolyl, furyl, thienyl, furanyl, pyridinyl, pyrazinyl, pyrimidinyl, pyridazinyl, napthyl, or pyrrolo[2,3-b]pyridinyl is substituted with 0, 1, 2, or 3 substituents independently selected from the group consisting of halo, hydroxy, haloalkyl, alkoxy, haloalkoxy, aryloxy, acylamino, amino, aminocarbonyl, carbonitrile, carboxyl ester, carboxyl, substituted sulfonyl, alkyl, substituted alkyl, heterocyclic, and substituted heterocyclic.

In some embodiments, each R^{3a} group is independently selected from the group consisting of F, Cl, Br, —NH₂, —NHOH, —NO₂, —CN, —CF₃,

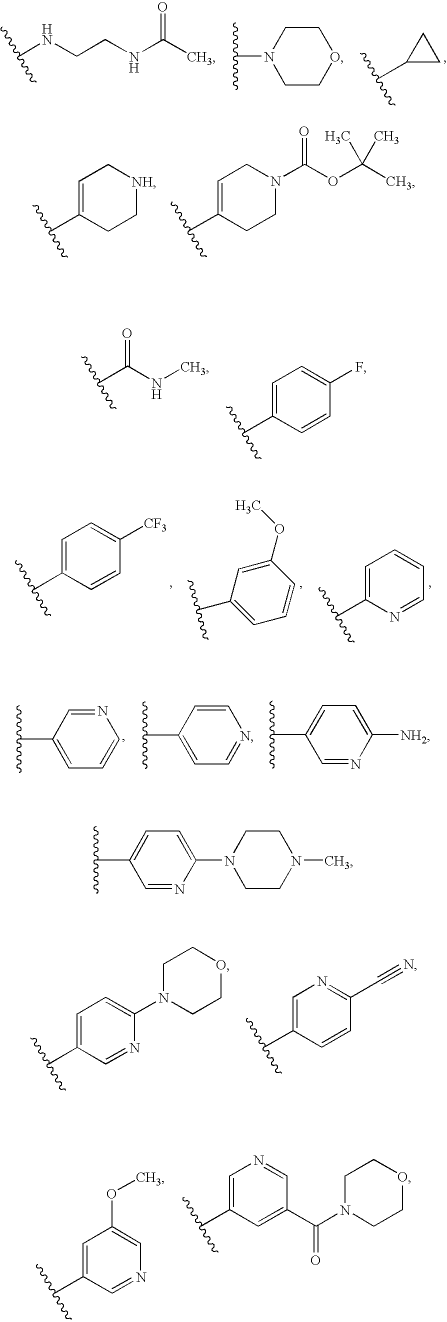

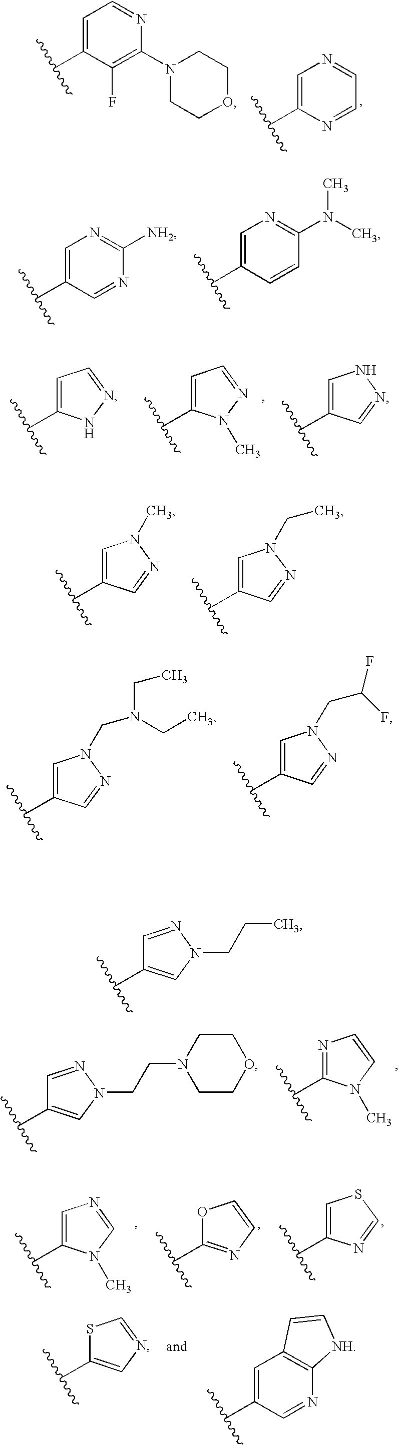

In some embodiments, two R^{3a} groups on two adjoining carbon atoms are taken together with the carbon atoms bound thereto to form a group selected from aryl, substituted aryl, heterocyclic, substituted heterocyclic, heteroaryl, and substituted heteroaryl.

In some embodiments, two R^{3a} groups on two adjoining carbon atoms are taken together with the carbon atoms bound thereto to form a benzene, thiophene, or pyrazole ring, wherein said benzene, thiophene, or pyrazole ring is substituted with 0, 1, 2, or 3 substituents independently selected from halo, hydroxy, alkyl, alkoxy.

In some embodiments, two R^{3a} groups on two adjoining carbon atoms are taken together with the carbon atoms bound thereto to form

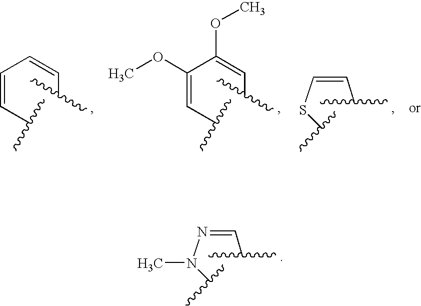

In some embodiments, ring A is

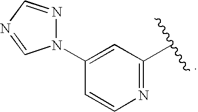

In some embodiments, ring A is selected from the group consisting of

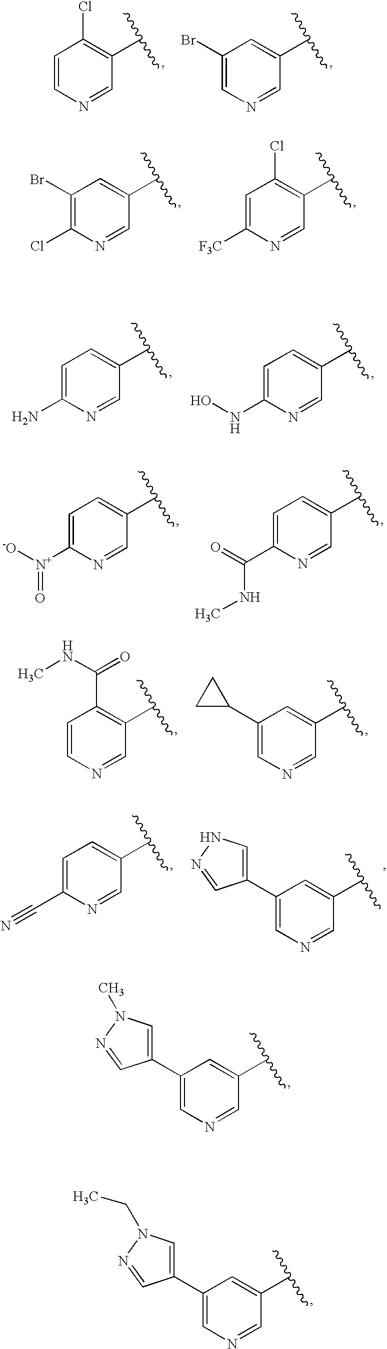

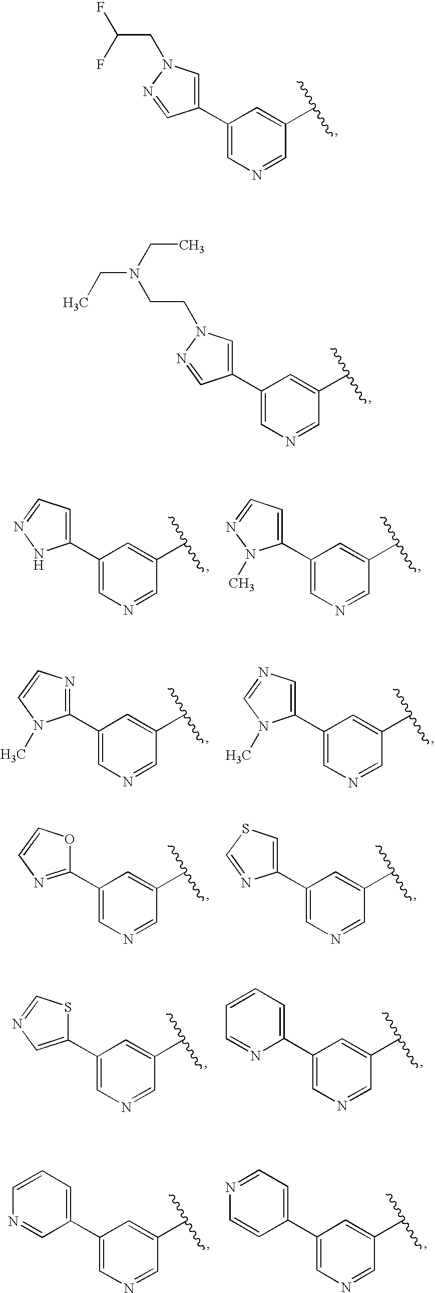

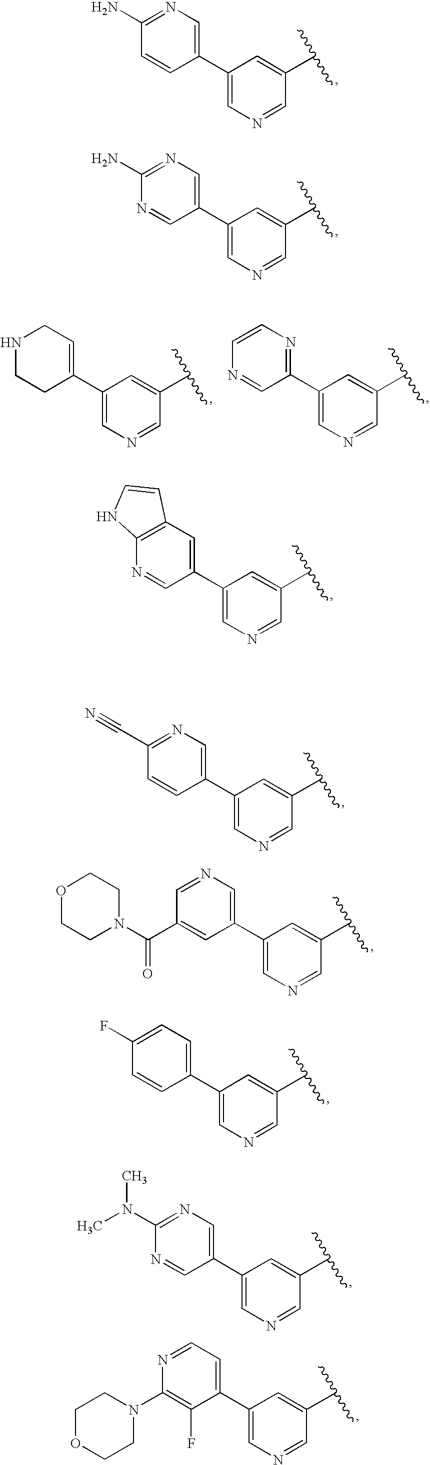

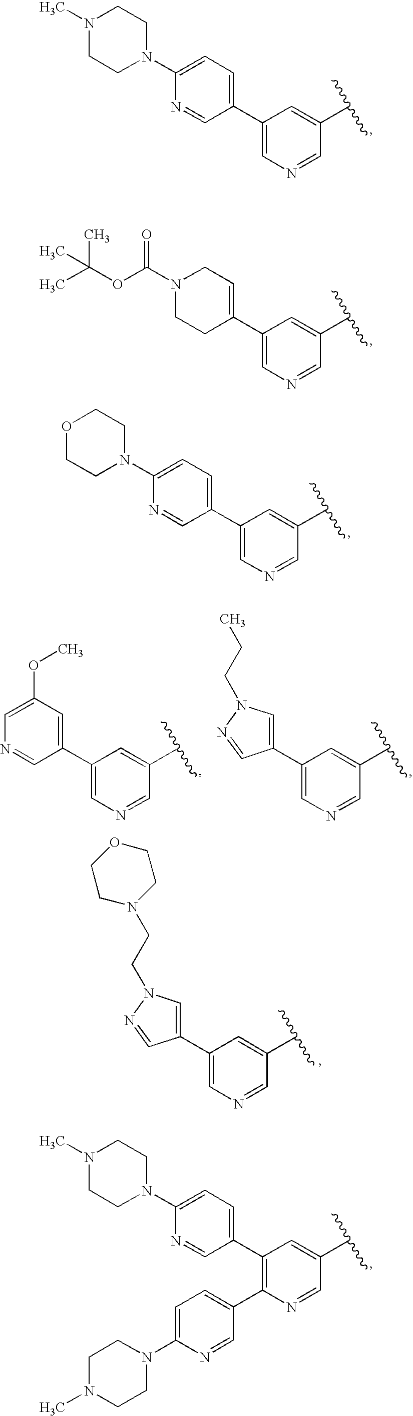

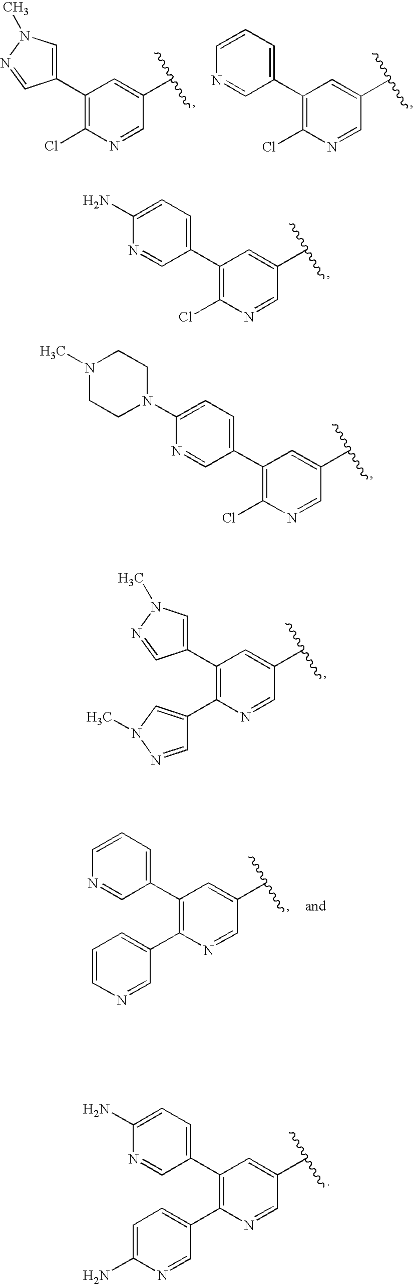

In some embodiments, ring A is

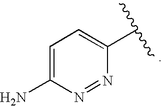

In some embodiments, ring A is selected from the group consisting of

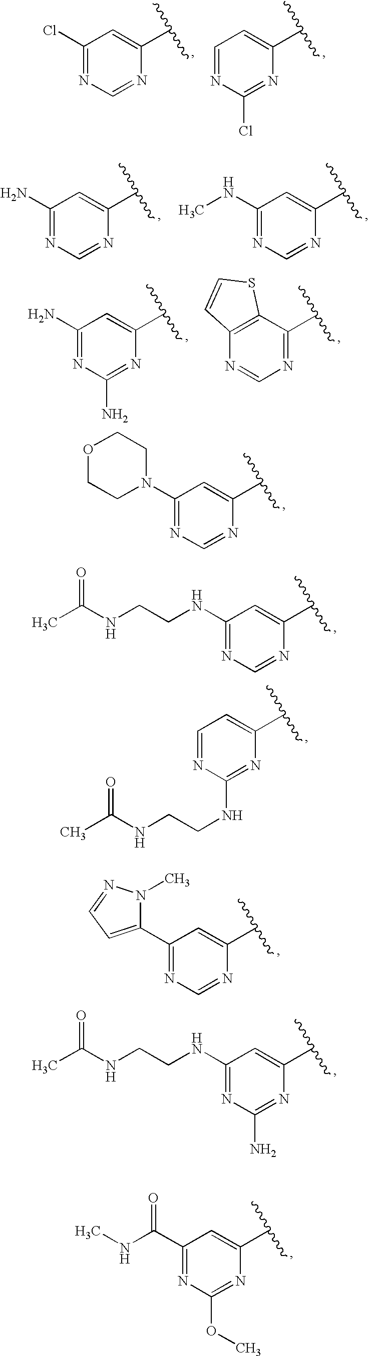

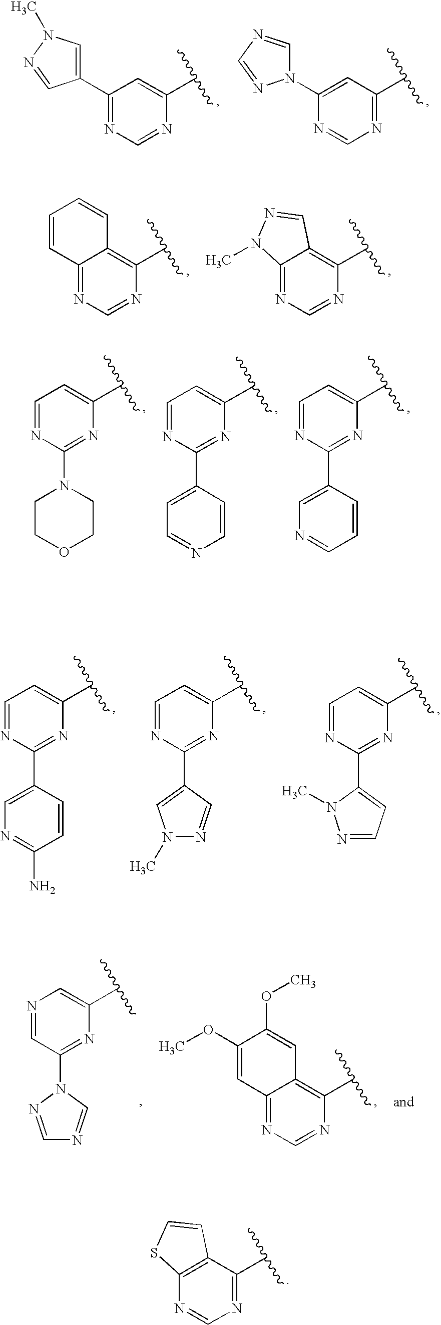

In some embodiments, ring A is selected from the group consisting of

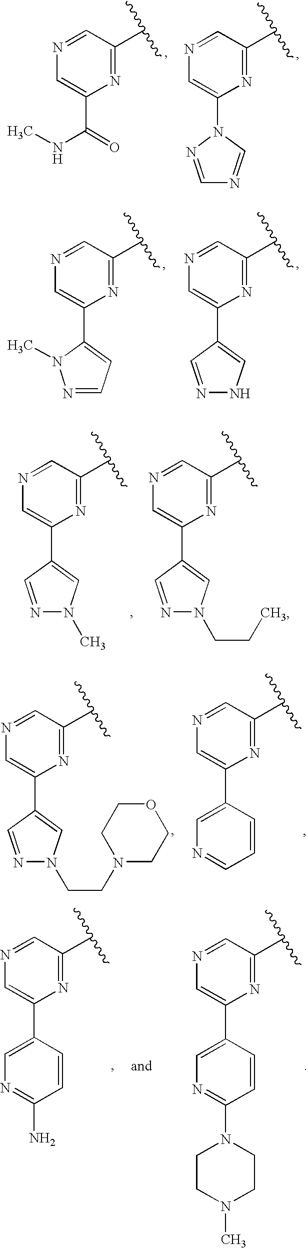

In some embodiments of its compound aspect, the invention provides compounds of Formula (VII):

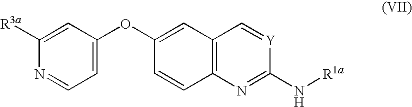

or an oxide, ester, prodrug, pharmaceutically acceptable salt, or solvate thereof,

wherein:

Y is N or CH;

R^{1a} is selected from the group consisting of hydrogen, alkyl, substituted alkyl, cycloalkyl, substituted cycloalkyl, cycloalkenyl, substituted cycloalkenyl, heterocyclyl, substituted heterocyclyl, acyl, and aminocarbonyl; and

R^{3a} is selected from the group consisting of halo, alkyl, substituted alkyl, alkenyl, substituted alkenyl, alkynyl, substituted alkynyl, carbonitrile, aryl, substituted aryl, cycloalkyl, substituted cycloalkyl, cycloalkenyl, substituted cycloalkenyl, heteroaryl, substituted heteroaryl, heterocyclic, substituted heterocyclic, amino, substituted amino, acylamino, alkoxy, substituted alkoxy, carboxyl, carboxyl ester, substituted sulfonyl, aminosulfonyl, and aminocarbonyl;

provided that R^{1a} is not:

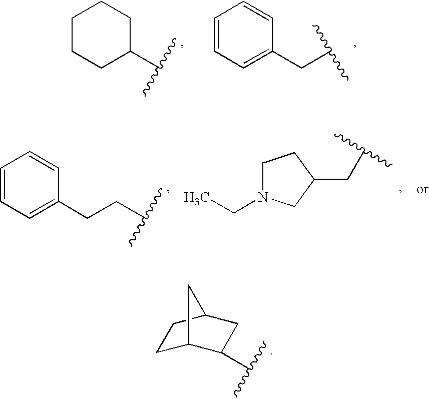

In some embodiments, R^{1a} is cycloalkyl, substituted cycloalkyl, heterocyclyl or substituted heterocyclyl.

In some embodiments, R^{1a} is cyclohexyl or cyclopentyl, wherein said cyclohexyl and cyclopentyl are optionally substituted with one to four substituents independently selected from the group consisting of hydroxy and amino; or two adjacent substituents join together to form a benzene ring fused with the cyclohexyl or cyclopentyl.

In some embodiments, R^{1a} is selected from the group consisting of:

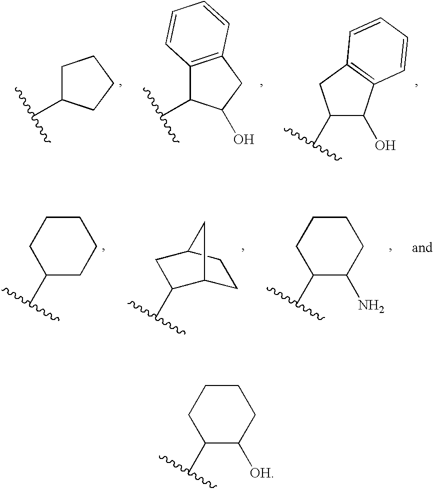

In some embodiments, R^{1a} is tetrahydropyran, piperidinyl or substituted piperidinyl.

In some embodiments, R^{1a} is alkyl or substituted alkyl.

In some embodiments, R^{1a} is alkyl substituted with one to four substituents selected from the group consisting of cycloalkyl, substituted cycloalkyl, hydroxy, phenyl, substituted phenyl, heterocyclyl, substituted heterocyclyl, heteroaryl and substituted heteroaryl.

In some embodiments, R^{1a} is alkyl substituted with at least one substituent selected from the group consisting of hydroxyl, cyclopropyl, cyclohexyl, morpholino, phenyl, substituted phenyl, thiazole and substituted thiazole.

In some embodiments, R^{1a} is —CO—R⁸ or —CO—NH—R⁸, wherein R⁸ is optionally substituted phenyl or optionally substituted cyclohexyl.

In some embodiments, R^{3a} is selected from hydrogen, aryl, substituted aryl, cycloalkyl, substituted cycloalkyl, cycloalkenyl, substituted cycloalkenyl, heteroaryl, substituted heteroaryl, heterocyclic, substituted heterocyclic, amino, substituted amino, acylamino, alkoxy, substituted alkoxy, carboxyl, carboxyl ester, substituted sulfonyl, aminosulfonyl, and aminocarbonyl.

In some embodiments, R^{3a} is selected from the group consisting of hydrogen, pyrazolyl, substituted pyrazolyl, imidazolyl, substituted imidazolyl, pyridinyl, substituted pyridinyl, acylamino and aminocarbonyl.

In some embodiments, R^{3a} group is independently selected from the group consisting of hydrogen,

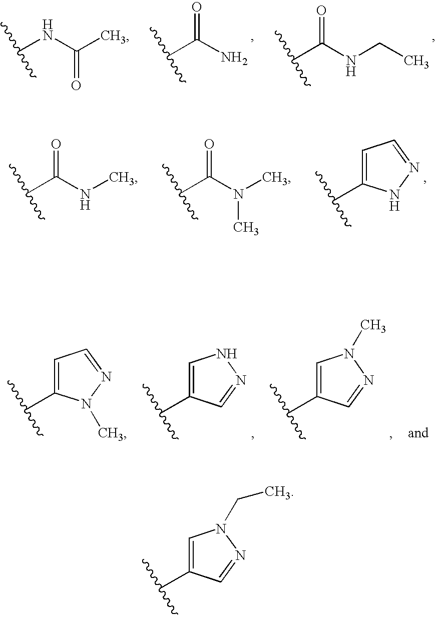

In some embodiments, provided is a compound selected from Tables 1-4 or an oxide, ester, prodrug, pharmaceutically acceptable salt, or solvate thereof is provided.

TABLE 1

| Cmpd # | Structure | Name |
|---|---|---|
| 84 | 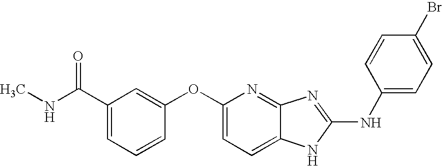 | 3-(2-(4-bromophenylamino)- 1H-imidazo[4,5-b]pyridin-5- yloxy)-N-methylbenzamide |
| 85 | 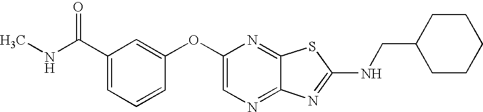 | 3-(2- (cyclohexylmethylamino)thiazolo [4,5-b]pyrazin-6-yloxy)-N- methylbenzamide |
| 79 | 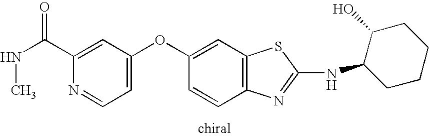 | 4-(2-((1R,2R)-2- hydroxycyclohexylamino) benzo[d]thiazol-6-ylamino)-N- methylpicolinamide |
| 81 | 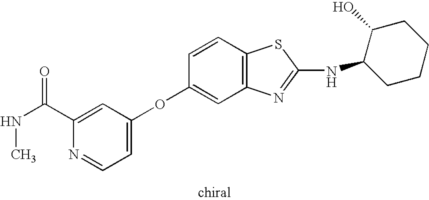 | 4-(2-((1R,2R)-2- hydroxycyclohexylamino) benzo[d]thiazol-5-yloxy)-N- methylpicolinamide |

TABLE 2

| Cmpd # | Structure | Name |
|---|---|---|
| 1 | 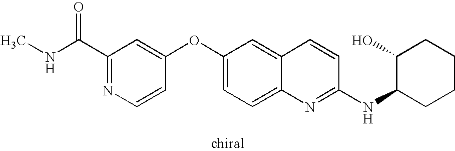 | 4-(2-((1R,2R)-2- hydroxycyclohexylamino) quinolin-6-yloxy)-N- methylpicolinamide |
| 2 | 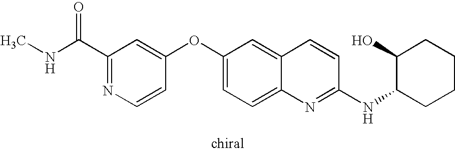 | 4-(2-((1S,2S)-2- hydroxycyclohexylamino) quinolin-6-yloxy)-N- methylpicolinamide |
| 3 | 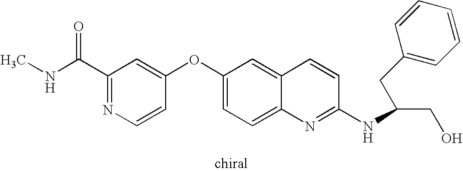 | (S)-4-(2-(1-hydroxy-3- phenylpropan-2- ylamino)quinolin-6-yloxy)-N- methylpicolinamide |
| 4 | 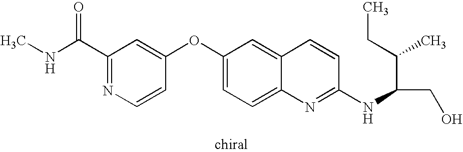 | 4-(2-((2S,3S)-1-hydroxy-3- methylpentan-2- ylamino)quinolin-6-yloxy)-N- methylpicolinamide |
| 5 | 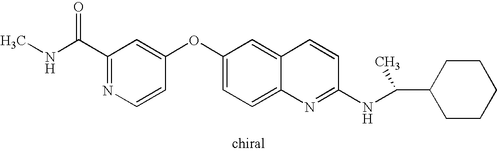 | (R)-4-(2-(1- cyclohexylethylamino)quinolin- 6-yloxy)-N- methylpicolinamide |
| 6 | 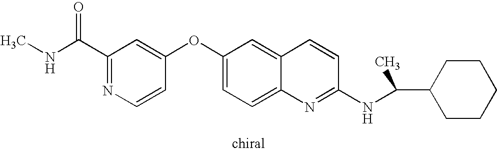 | (S)-4-(2-(1- cyclohexylethylamino)quinolin- 6-yloxy)-N- methylpicolinamide |
| 7 | 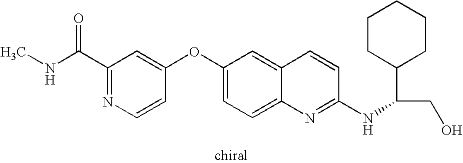 | (R)-4-(2-(1-cyclohexyl-2- hydroxyethylamino)quinolin-6- yloxy)-N-methylpicolinamide |
| 8 | 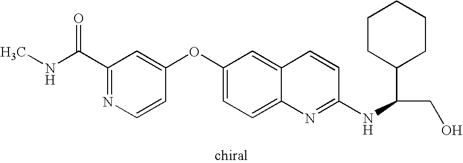 | (S)-4-(2-(1-cyclohexyl-2- hydroxyethylamino)quinolin-6- yloxy)-N-methylpicolinamide |
| 9 | 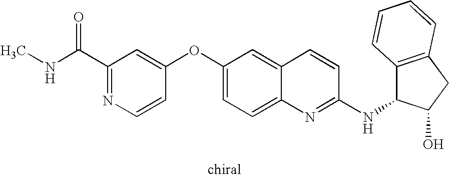 | 4-(2-((1R,2S)-2-hydroxy-2,3- dihydro-1H-inden-1- ylamino)quinolin-6-yloxy)-N- methylpicolinamide |
| 10 | 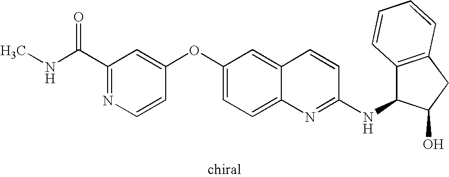 | 4-(2-((1S,2R)-2-hydroxy-2,3- dihydro-1H-inden-1- ylamino)quinolin-6-yloxy)-N- methylpicolinamide |
| 11 | 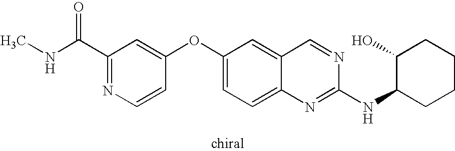 | 4-(2-((1R,2R)-2- hydroxycyclohexylamino) quinazolin-6-yloxy)-N- methylpicolinamide |
| 12 | 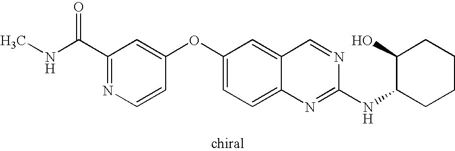 | 4-(2-((1S,2S)-2- hydroxycyclohexylamino) quinazolin-6-yloxy)-N- methylpicolinamide |
| 13 | 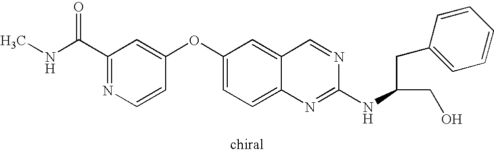 | (S)-4-(2-(1-hydroxy-3- phenylpropan-2- ylamino)quinazolin-6-yloxy)- N-methylpicolinamide |
| 14 | 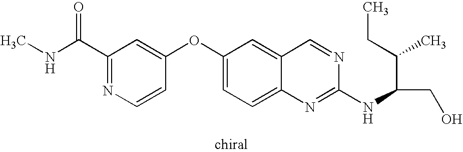 | 4-(2-((2S,3S)-1-hydroxy-3- methylpentan-2- ylamino)quinazolin-6-yloxy)- N-methylpicolinamide |
| 15 | 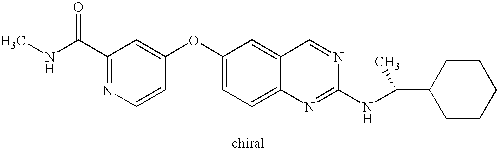 | (R)-4-(2-(1- cyclohexylethylamino)quinazolin- 6-yloxy)-N- methylpicolinamide |
| 16 | 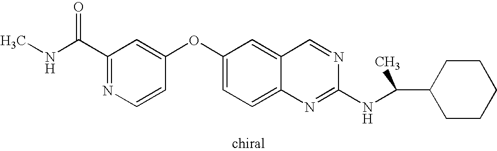 | (S)-4-(2-(1- cyclohexylethylamino)quinazolin- 6-yloxy)-N- methylpicolinamide |
| 17 | 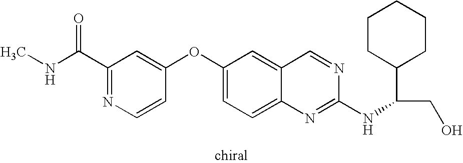 | (R)-4-(2-(1-cyclohexyl-2- hydroxyethylamino)quinazolin- 6-yloxy)-N- methylpicolinamide |
| 18 | 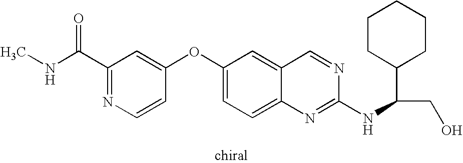 | (S)-4-(2-(1-cyclohexyl-2- hydroxyethylamino)quinazolin- 6-yloxy)-N- methylpicolinamide |
| 19 | 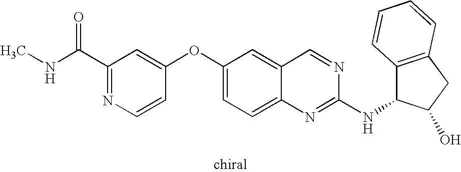 | 4-(2-((1R,2S)-2-hydroxy-2,3- dihydro-1H-inden-1- ylamino)quinazolin-6-yloxy)- N-methylpicolinamide |
| 20 | 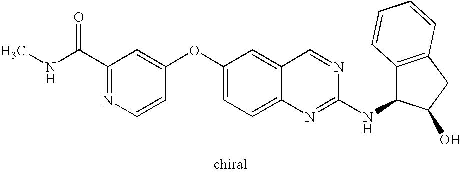 | 4-(2-((1S,2R)-2-hydroxy-2,3- dihydro-1H-inden-1- ylamino)quinazolin-6-yloxy)- N-methylpicolinamide |
| 21 | 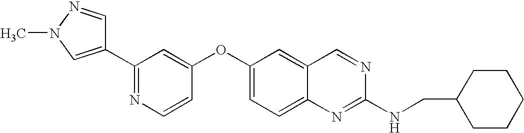 | N-(cyclohexylmethyl)-6-(2-(1- methyl-1H-pyrazol-4- yl)pyridin-4-yloxy)quinazolin- 2-amine |
| 35 | 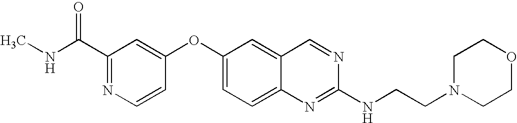 | N-methyl-4-(2-(2- morpholinoethylamino) quinazolin- 6-yloxy)picolinamide |
| 37 | 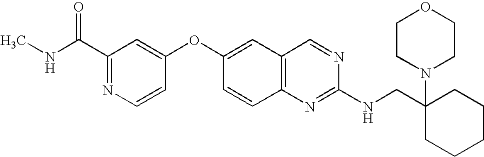 | N-methyl-4-(2-((1- morpholinocyclohexyl) methylamino)quinazolin-6- yloxy)picolinamide |
| 38 | 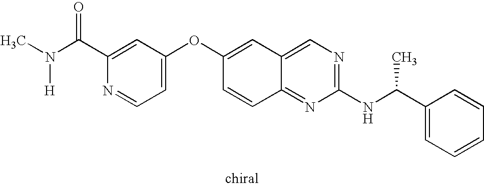 | (R)-N-methyl-4-(2-(1- phenylethylamino)quinazolin- 6-yloxy)picolinamide |
| 39 | 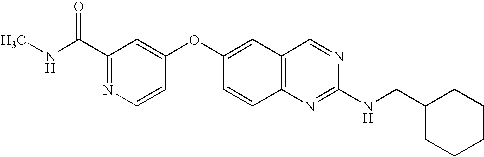 | 4-(2- (cyclohexylmethylamino) quinazolin-6-yloxy)-N- methylpicolinamide |
| 41 | 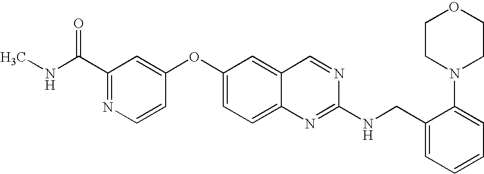 | N-methyl-4-(2-(2- morpholinobenzylamino) quinazolin-6-yloxy)picolinamide |
| 42 | 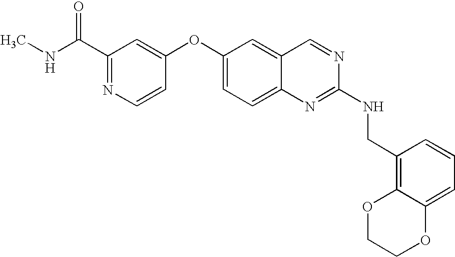 | 4-(2-((2,3- dihydrobenzo[b][1,4]dioxin-5- yl)methylamino)quinazolin-6- yloxy)-N-methylpicolinamide |
| 43 | 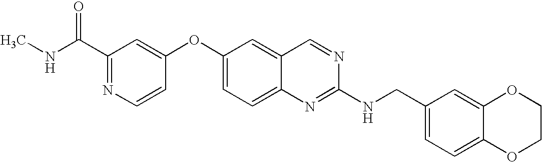 | 4-(2-((2,3- dihydrobenzo[b][1,4]dioxin-6- yl)methylamino)quinazolin-6- yloxy)-N-methylpicolinamide |
| 48 | 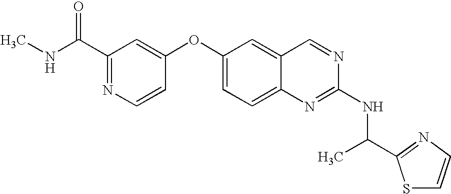 | N-methyl-4-(2-(1-(thiazol-2- yl)ethylamino)quinazolin-6- yloxy)picolinamide |
| 52 | 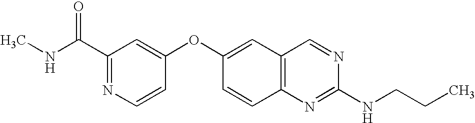 | N-methyl-4-(2- (propylamino)quinazolin-6- yloxy)picolinamide |
| 53 | 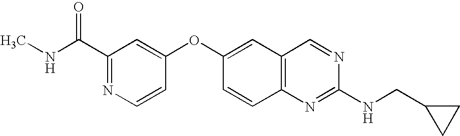 | 4-(2- (cyclopropylmethylamino) quinazolin-6-yloxy)-N- methylpicolinamide |
| 56 | 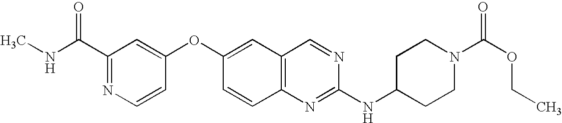 | ethyl 4-(6-(2- (methylcarbamoyl)pyridin-4- yloxy)quinazolin-2- ylamino)piperidine-1- carboxylate |
| 57 | 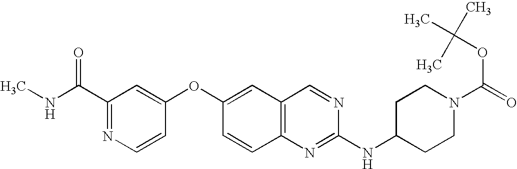 | tert-butyl 4-(6-(2- (methylcarbamoyl)pyridin-4- yloxy)quinazolin-2- ylamino)piperidine-1- carboxylate |
| 58 | 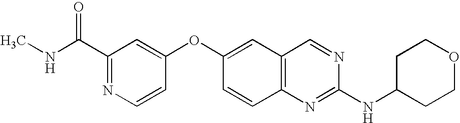 | N-methyl-4-(2-(tetrahydro-2H- pyran-4-ylamino)quinazolin-6- yloxy)picolinamide |
| 59 | 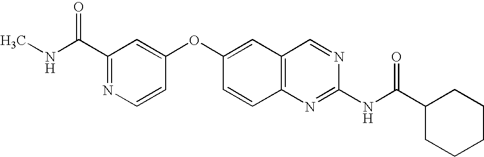 | 4-(2- (cyclohexanecarboxamido) quinazolin-6-yloxy)-N- methylpicolinamide |
| 60 | 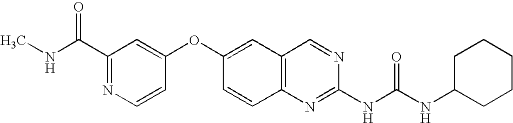 | 4-(2-(3- cyclohexylureido)quinazolin-6- yloxy)-N-methylpicolinamide |
| 61 | 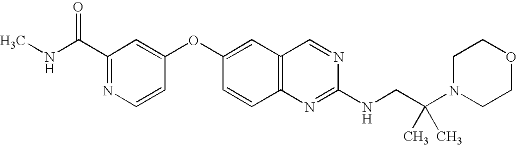 | N-methyl-4-(2-(2-methyl-2- morpholinopropylamino) quinazolin-6-yloxy)picolinamide |
| 63 | 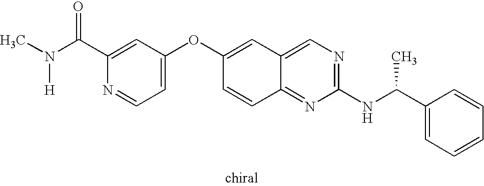 | (R)-N-methyl-4-(2-(1- phenylethylamino)quinazolin- 6-yloxy)picolinamide |
| 64 | 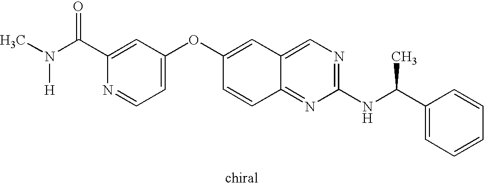 | (S)-N-methyl-4-(2-(1- phenylethylamino)quinazolin- 6-yloxy)picolinamide |
| 66 | 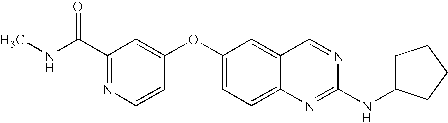 | 4-(2- (cyclopentylamino)quinazolin- 6-yloxy)-N- methylpicolinamide |
| 72 | 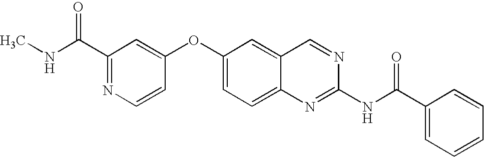 | 4-(2-benzamidoquinazolin-6- yloxy)-N-methylpicolinamide |
| 73 | 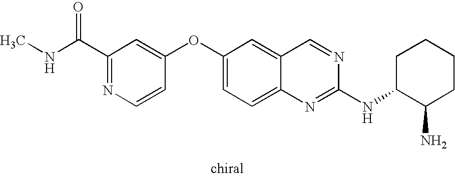 | 4-(2-((1R,2R)-2- aminocyclohexylamino) quinazolin-6-yloxy)-N- methylpicolinamide |
| 74 | 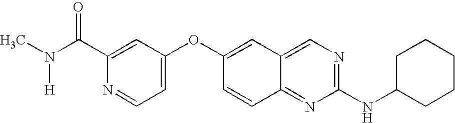 | 4-(2- (cyclohexylamino)quinazolin- 6-yloxy)-N- methylpicolinamide |

TABLE 3

| Cmpd # | Structure | Name |
|---|---|---|
| 28 | 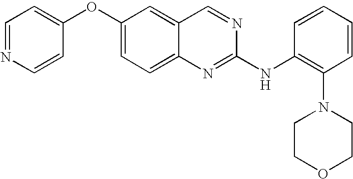 | N-(2-morpholinophenyl)-6- (pyridin-4-yloxy)quinazolin-2- amine |
| 29 | 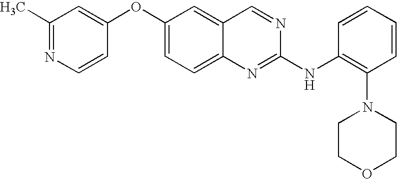 | 6-(2-aminopyridin-4-yloxy)- N-(2- morpholinophenyl)quinazolin- 2-amine |
| 31 | 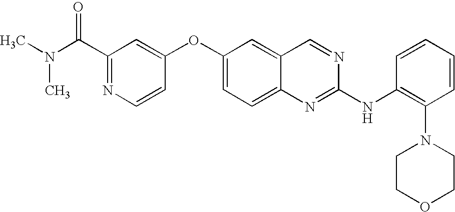 | N,N-dimethyl-4-(2-(2- morpholinophenylamino) quinazolin-6-yloxy)picolinamide |
| 32 | 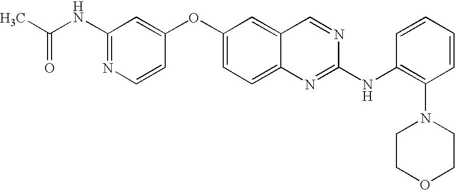 | N-(4-(2-(2- morpholinophenylamino) quinazolin-6-yloxy)pyridin-2- yl)acetamide |
| 33 | 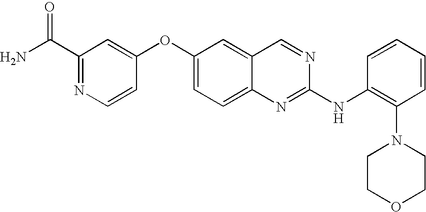 | 4-(2-(2- morpholinophenylamino) quinazolin-6-yloxy)picolinamide |
| 34 | 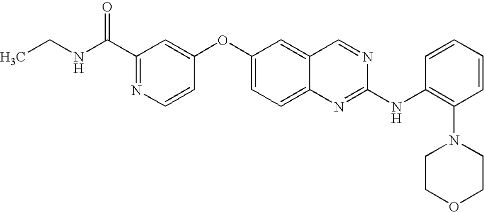 | N-ethyl-4-(2-(2- morpholinophenylamino) quinazolin-6-yloxy)picolinamide |
| 40 | 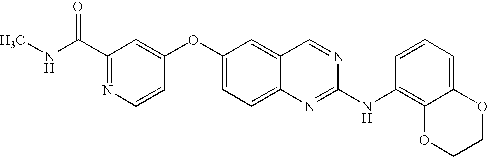 | 4-(2-(2,3- dihydrobenzo[b][1,4]dioxin-5- ylamino)quinazolin-6-yloxy)- N-methylpicolinamide |
| 44 | 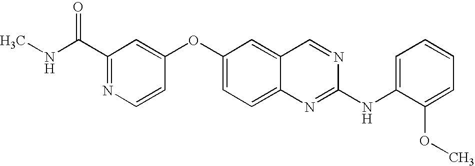 | 4-(2-(2- methoxyphenylamino) quinazolin-6-yloxy)-N- methylpicolinamide |
| 45 | 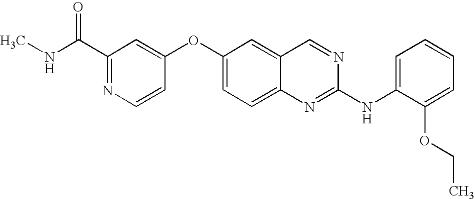 | 4-(2-(2- ethoxyphenylamino) quinazolin-6-yloxy)-N- methylpicolinamide |
| 46 | 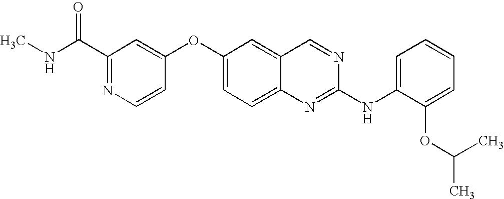 | 4-(2-(2- isopropoxyphenylamino) quinazolin-6-yloxy)-N- methylpicolinamide |
| 54 | 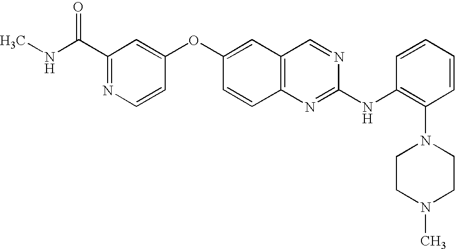 | N-methyl-4-(2-(2-(4- methylpiperazin-1- yl)phenylamino)quinazolin-6- yloxy)picolinamide |
| 55 | 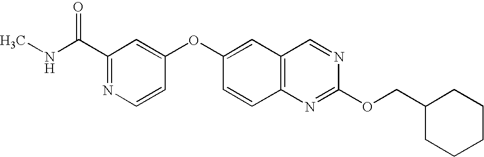 | 4-(2- (cyclohexylmethoxy) quinazolin-6-yloxy)-N- methylpicolinamide |
| 67 | 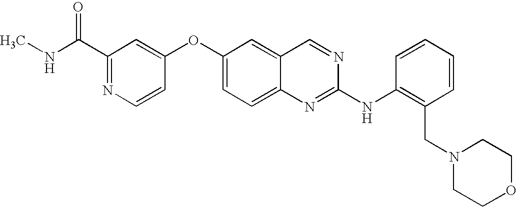 | N-methyl-4-(2-(2- (morpholinomethyl) phenylamino)quinazolin-6- yloxy)picolinamide |
| 68 | 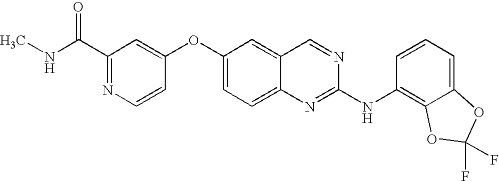 | 4-(2-(2,2- difluorobenzo[d][1,3]dioxol-4- ylamino)quinazolin-6-yloxy)- N-methylpicolinamide |
| 69 | 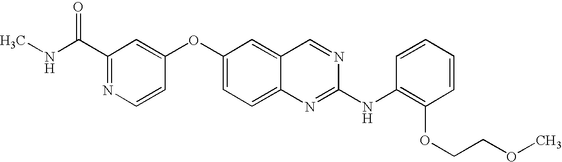 | 4-(2-(2-(2- methoxyethoxy)phenylamino) quinazolin-6-yloxy)-N- methylpicolinamide |
| 70 | 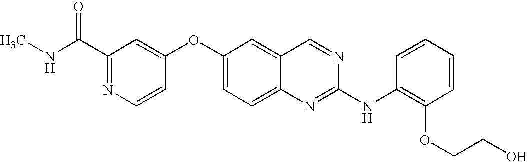 | 4-(2-(2-(2- hydroxyethoxy)phenylamino) quinazolin-6-yloxy)-N- methylpicolinamide |
| 71 | 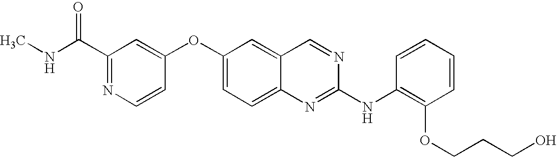 | 4-(2-(2-(3- hydroxypropoxy)phenylamino) quinazolin-6-yloxy)-N- methylpicolinamide |
| 86 | 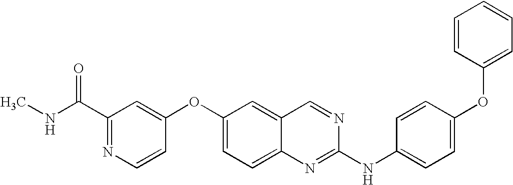 | N-methyl-4-(2-(4- phenoxyphenylamino) quinazolin-6-yloxy)picolinamide |
| 87 | 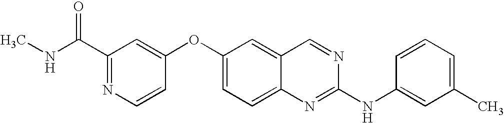 | N-methyl-4-(2-(m- tolylamino)quinazolin-6- yloxy)picolinamide |

TABLE 4

| Cmpd # | Structure | Name |
|---|---|---|
| 76 | 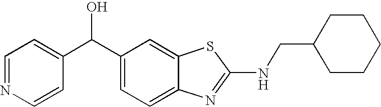 | (2- (cyclohexylmethylamino)benzo [d]thiazol-6-yl)(pyridin-4- yl)methanol |
| 77 | 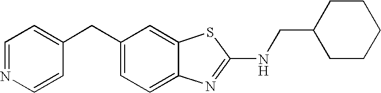 | N-(cyclohexylmethyl)-6- (pyridin-4- ylmethyl)benzo[d]thiazol-2- amine |
| 78 | 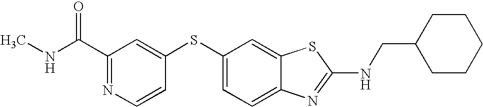 | 4-(2- (cyclohexylmethylamino)benzo [d]thiazol-6-ylthio)-N- methylpicolinamide |
| 82 | 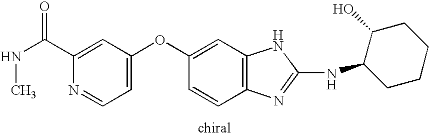 | 4-(2-((1R,2R)-2- hydroxycyclohexylamino)-1H- benzo[d]imidazol-6-yloxy)-N- methylpicolinamide |
| 83 | 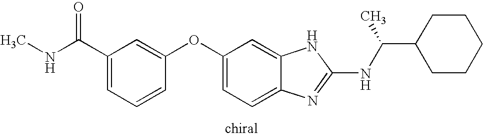 | (R)-4-(2-(1- cyclohexylethylamino)-1H- benzo[d]imidazol-6-yloxy)-N- methylpicolinamide |

In some embodiments, provided is a pharmaceutical composition effective to inhibit CSF-1R activity in a human or animal subject when administered thereto, comprising a therapeutically effective amount of a compound of the invention including the compounds of any one of Formulas (II), (III), (IV), (V), (VI), and (VII), or of any one of Tables 1-4, or an oxide, ester, prodrug, solvate, or pharmaceutically acceptable salts thereof and a pharmaceutically acceptable carrier.

It will also be apparent to those skilled in the art that the compounds of the invention, including the compounds of any one of Formulas (I), (II), (III), (IV), (V), (VI), and (VII), or of any one of Tables 1-4, or the pharmaceutically acceptable salts, esters, oxides, and prodrugs of any of them, may be subject to tautomerization and may therefore exist in various tautomeric forms.

Compounds of any one of Formulas (I), (II), (III), (IV), (V), (VI), and (VII), or of any one of Tables 1-4, as well as the pharmaceutically acceptable salts, esters, oxides, and prodrugs of any of them, may comprise asymmetrically substituted carbon atoms. Such asymmetrically substituted carbon atoms can result in the compounds existing in enantiomers, diastereomers, and other stereoisomeric forms that may be defined, in terms of absolute stereochemistry, such as in (R)- or (S)-forms. As a result, all such possible isomers, individual stereoisomers in their optically pure forms, mixtures thereof, racemic mixtures (or “racemates”), mixtures of diastereomers, as well as single diastereomers of the compounds are contemplated. The terms “S” and “R” configuration, as used herein, are as defined by the IUPAC 1974 RECOMMENDATIONS FOR SECTION E, FUNDAMENTAL STEREOCHEMISTRY, Pure Appl. Chem. 45:13-30 (1976).

### Methods for Treating CSF-1R Mediated Diseases

There are three distinct mechanisms by which CSF-1R signaling is likely involved in tumor growth and metastasis. The first is that expression of CSF-ligand and receptor has been found in tumor cells originating in the female reproductive system (breast, ovarian, endometrium, cervical) (Scholl 1994; Kacinski 1997; Nagan 199; Kirma 2007) and the expression has been associated with breast cancer xenograft growth as well as poor prognosis in breast cancer patients. Two point mutations were seen in CSF-1R in about 10-20% of acute myelocytic leukemia, chronic myelocytic leukemia and myelodysplasia patients tested in one study, and one of the mutations was found to disrupt receptor turnover (Ridge 1990). However the incidence of the mutations could not be confirmed in later studies (Abu-Duhier 2003). Mutations were also found in some cases of hepatocellular cancer (Yang 2004) and idiopathic myelofibrosis (Abu-Duhier 2003).

Pigmented villonodular synovitis (PVNS) and Tenosynovial Giant cell tumors (TGCT) can occur as a result of a translocation that fuses the M-CSF gene to a collagen gene COL6A3 and results in overexpression of M-CSF (West 2006). A landscape effect is proposed to be responsible for the resulting tumor mass that consists of monocytic cells attracted by cells that express M-CSF. TGCTs are smaller tumors that can be relatively easily removed from fingers where they mostly occur. PVNS is more aggressive as it can recur in large joints and is not as easily controlled surgically.

The second mechanism is based on blocking signaling through M-CSF/CSF-1R at metastatic sites in bone which induces osteoclastogenesis, bone resorption and osteolytic bone lesions. Breast, kidney, and lung cancers are examples of cancers that have been found to metastasize to the bone and cause osteolytic bone disease resulting in skeletal complications. M-CSF released by tumor cells and stroma induces the differentiation of hematopoietic myeloid monocyte progenitors to mature osteoclasts in collaboration with the receptor activator of nuclear factor kappa-B ligand-RANKL. During this process, M-CSF acts as a permissive factor by giving the survival signal to osteoclasts (Tanaka 1993) Inhibition of CSF-1R kinase activity during osteoclast differentiation and maturation with a small molecule inhibitor is likely to prevent unbalanced activity of osteoclasts that cause osteolytic disease and the associated skeletal related events in metastatic disease. Whereas breast, lung cancer and multiple myeloma typically result in osteolytic lesions, metastasis to the bone in prostate cancer initially has an osteoblastic appearance in which increased bone forming activity results in ‘woven bone’ which is different from typical lamellar structure of normal bone. During disease progression bone lesions display a significant osteolytic component as well as high serum levels of bone resorption and suggests that anti-resorptive therapy may be useful. Bisphosphonates have been shown to inhibit the formation of osteolytic lesions and reduced the number of skeletal-related events only in men with hormone-refractory metastatic prostate cancer but at this point their effect on osteoblastic lesions is controversial and bisphosphonates have not been beneficial in preventing bone metastasis or hormone responsive prostate cancer to date. The effect of anti-resorptive agents in mixed osteolytic/osteoblastic prostate cancer is still being studied in the clinic (Choueiri 2006; Vessella 2006).

The third mechanism is based on the recent observation that tumor associated macrophages (TAM) found in solid tumors of the breast, prostate, ovarian and cervical cancers correlated with poor prognosis (Bingle 2002; Pollard 2004). Macrophages are recruited to the tumor by M-CSF and other chemokines. The macrophages can then contribute to tumor progression through the secretion of angiogenic factors, proteases and other growth factors and cytokines and may be blocked by inhibition of CSF-1R signaling. Recently it was shown by Zins et al (Zins 2007) that expression of siRNA of Tumor necrosis factor alpha (TNFα), M-CSF or the combination of both would reduce tumor growth in a mouse xenograft model between 34% and 50% after intratumoral injection of the respective siRNA into the xenograft. siRNA targeting the TNFα secreted by the human SW620 cells reduced the mouse M-CSF and led to reduction of macrophages in the tumor. In addition, treatment of MCF-7 tumor xenografts with an antigen binding fragment directed against M-CSF antibody resulted in 40% tumor growth inhibition, reversed the resistance to chemotherapeutics and improved survival of the mice when given in combination with chemotherapeutics (Paulus 2006).

TAMs are only one example of an emerging link between chronic inflammation and cancer. There is additional evidence for a link between inflammation and cancer as many chronic diseases are associated with an increased risk of cancer, cancers arise at sites of chronic inflammation and chemical mediators of inflammation are found in many cancers; deletion of the cellular or chemical mediators of inflammation inhibits development of experimental cancers and long-term use of anti-inflammatory agents reduce the risk of some cancers. A link to cancer exists for a number of inflammatory conditions among those H. pylori induced gastritis for gastric cancer, Schistosomiasis for bladder cancer, HHV8 for Kaposi's sarcoma, endometriosis for ovarian cancer and prostatitis for prostate cancer (Balkwill 2005). Macrophages are key cells in chronic inflammation and respond differentially to their microenvironment. There are two types of macrophages that are considered extremes in a continuum of functional states: M1 macrophages are involved in Type 1 reactions. These reactions involve the activation by microbial products and consequent killing of pathogenic microorganisms that result in reactive oxygen intermediates. On the other end of the extreme are M2 macrophages involved in Type 2 reactions that promote cell proliferation, tune inflammation and adaptive immunity and promote tissue remodeling, angiogenesis and repair (Mantovani 2004). Chronic inflammation resulting in established neoplasia is usually associated with M2 macrophages. A pivotal cytokine that mediates inflammatory reactions is TNF-a that true to its name can stimulate anti-tumor immunity and hemorrhagic necrosis at high doses but has also recently been found to be expressed by tumor cells and acting as a tumor promoter (Zins 2007; Balkwill 2006). The specific role of macrophages with respect to the tumor still needs to be better understood including the potential spatial and temporal dependence on their function and the relevance to specific tumor types.

In another embodiment, a method for treating periodontitis, histiocytosis X, osteoporosis, Paget's disease of bone (PDB), bone loss due to cancer therapy, periprosthetic osteolysis, glucocorticoid-induced osteoporosis, rheumatoid arthritis, psoriatic arthritis, osteoarthritis, inflammatory arthridities, and inflammation is provided.

Rabello 2006 has demonstrated that SNPs in the CSF1 gene exhibited a positive association with aggressive periodontitis: an inflammatory disease of the periodontal tissues that causes tooth loss due to resorption of the alveolar bone.

Histiocytosis X (also called Langerhans cell histiocytosis, LCH) is a proliferative disease of Langerhans dendritic cells that appear to differentiate into osteoclasts in bone and extraosseous LCH lesions. Langerhans cells are derived from circulating monocytes (Ginoux 2006). Increased levels of M-CSF that have been measured in sera and lesions where found to correlate with disease severity (da Costa 2005). The disease occurs primarily in a pediatric patient population and has to be treated with chemotherapy when the disease becomes systemic or is recurrent.

The pathophysiology of osteoporosis is mediated by loss of bone forming osteoblasts and increased osteoclast dependent bone resorption. Supporting data has been described by Cenci et al showing that an anti-M-CSF antibody injection preserves bone density and inhibits bone resorption in ovarectomized mice (Cenci 2000). Recently a potential link between postmenopausal bone loss due to estrogen deficiency was identified and found that the presence of TNFα-producing T-cell affected bone metabolism (Roggia 2004). A possible mechanism could be the induction of M-CSF by TNFα in vivo. An important role for M-CSF in TNFα-induced osteoclastogenesis was confirmed by the effect of an antibody directed against the M-CSF-inhibitor that blocked the TNFα-induced osteolysis in mice and thereby making inhibitors of CSF-1R signaling potential targets for inflammatory arthritis (Kitaura 2005).

Paget's disease of bone (PDB) is the 2^{nd} most common bone metabolism disorder after osteoporosis in which focal abnormalities of increased bone turnover lead to complications such as bone pain, deformity, pathological fractures, and deafness. Mutations in four genes have been identified that regulate normal osteoclast function and predispose individuals to PDB and related disorders: insertion mutations in TNFRSF11A, which encodes receptor activator of nuclear factor (NF) kappaB (RANK)—a critical regulator of osteoclast function, inactivating mutations of TNFRSF11B which encodes osteoprotegerin (a decoy receptor for RANK ligand), mutations of the sequestosome 1 gene (SQSTM1), which encodes an important scaffold protein in the NFkappaB pathway and mutations in the valosin-containing protein (VCP) gene. This gene encodes VCP, which has a role in targeting the inhibitor of NFkappaB for degradation by the proteasome (Daroszewska, 2006). Targeted CSF-1R inhibitors provide an opportunity to block the deregulation of the RANKL signaling indirectly and add an additional treatment option to the currently used bisphosphonates.

Cancer therapy induced bone loss especially in breast and prostate cancer patients is an additional indication where a targeted CSF-1R inhibitor could prevent bone loss (Lester 2006). With the improved prognosis for early breast cancer the long-term consequences of the adjuvant therapies become more important as some of the therapies including chemotherapy, irradiation, aromatase inhibitors and ovary ablation affect bone metabolism by decreasing the bone mineral density, resulting in increased risk for osteoporosis and associated fractures (Lester 2006). The equivalent to adjuvant aromatase inhibitor therapy in breast cancer is androgen ablation therapy in prostate cancer which leads to loss of bone mineral density and significantly increases the risk of osteoporosis-related fractures (Stoch 2001).

Targeted inhibition of CSF-1R signaling is likely to be beneficial in other indications as well when targeted cell types include osteoclasts and macrophages e.g. treatment of specific complications in response to joint replacement as a consequence of rheumatoid arthritis. Implant failure due to periprosthetic bone loss and consequent loosing of protheses is a major complication of joint replacement and requires repeated surgery with high socioeconomic burdens for the individual patient and the health-care system. To date, there is no approved drug therapy to prevent or inhibit periprosthetic osteolysis (Drees 2007).

Glucocorticoid-induced osteoporosis (GIOP) is another indication in which a CSF-1R inhibitor could prevent bone loss after long-term glucocorticocosteroid use that is given as a result of various conditions among those chronic obstructive pulmonary disease, asthma and rheumatoid arthritis (Guzman-Clark 2007; Feldstein 2005).

Rheumatoid arthritis, psoriatic arthritis and inflammatory arthridities are in themselves potential indications for CSF-1R signaling inhibitors in that they consist of a macrophage component, and to a varying degree bone destruction (Ritchlin 2003). Osteoarthritis and rheumatoid arthritis are inflammatory autoimmune diseases caused by the accumulation of macrophages in the connective tissue and infiltration of macrophages into the synovial fluid, which is at least partially mediated by M-CSF. Campbell et al. (2000) demonstrated that M-CSF is produced by human-joint tissue cells (chondrocytes, synovial fibroblasts) in vitro and is found in synovial fluid of patients with rheumatoid arthritis, suggesting that it contributes to the synovial tissue proliferation and macrophage infiltration which is associated with the pathogenesis of the disease Inhibition of CSF-1R signaling is likely to control the number of macrophages in the joint and alleviate the pain from the associated bone destruction. In order to minimize adverse affects and to further understand the impact of the CSF-1R signaling in these indications, one method is to specifically inhibit CSF-1R without targeting a myriad other kinases, such as Raf kinase.

Recent literature reports correlate increased circulating M-CSF with poor prognosis and atherosclerotic progression in chronic coronary artery disease (Saitoh 2000; Ikonomidis 2005); M-CSF influences the atherosclerotic process by aiding the formation of foam cells (macrophages with ingested oxidized LDL) that express CSF-1R and represent the initial plaque (Murayama 1999).

Expression and signaling of M-CSF and CSF-1R is found in activated microglia. Microglia, which are resident macrophages of the central nervous system, can be activated by various insults, including infection and traumatic injury. M-CSF is considered a key regulator of inflammatory responses in the brain and M-CSF levels increase in HIV-1 encephalitis, Alzheimer's disease (AD) and brain tumors. Microgliosis as a consequence of autocrine signaling by M-CSF/CSF-1R results in induction of inflammatory cytokines and nitric oxides being released as demonstrated by e.g. using an experimental neuronal damage model (Hao 2002; Murphy 1998). Microglia that have increased expression of CSF-1R are found to surround plaques in AD and in the amyloid precursor protein V717F transgenic mouse model of AD (Murphy 2000). On the other hand op/op mice with fewer microglia in the brain resulted in fibrilar deposition of Aβ and neuronal loss compared to normal control suggesting that microglia do have a neuroprotective function in the development of AD lacking in the op/op mice (Kaku 2003).

In some embodiments of its method aspect, the present invention provides a method for treating a CSF-1R mediated disorder in a patient, comprising administering to the patient a compound of Formula (I):

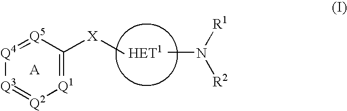

or an oxide, ester, prodrug, pharmaceutically acceptable salt, or solvate thereof, wherein:

A is a six-member ring where each of Q¹, Q², Q³, Q⁴ and Q⁵ is independently C—R³ or N, provided that at least one of Q¹, Q², Q³, Q⁴ and Q⁵ is N and at most three of Q¹, Q², Q³, Q⁴ and Q⁵ are N;

each R³ is independently hydrogen or R^{3a}, where R^{3a} is selected from the group consisting of halo, alkyl, substituted alkyl, alkenyl, substituted alkenyl, alkynyl, substituted alkynyl, carbonitrile, aryl, substituted aryl, cycloalkyl, substituted cycloalkyl, cycloalkenyl, substituted cycloalkenyl, heteroaryl, substituted heteroaryl, heterocyclic, substituted heterocyclic, amino, substituted amino, acylamino, alkoxy, substituted alkoxy, carboxyl, carboxyl ester, substituted sulfonyl, aminosulfonyl, and aminocarbonyl; or two adjacent R^{3a} groups together form a aryl, substituted aryl, heterocyclic, substituted heterocyclic, heteroaryl, or substituted heteroaryl group that is fused to ring A;

HET¹ is a bicyclic ring selected from the group consisting of:
(a) a [6,6] fused bicyclic ring selected from the group consisting of:

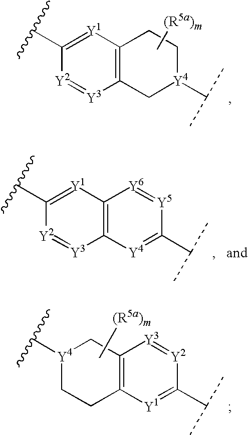

(b) a [5,6] fused bicyclic ring selected from the group consisting of:

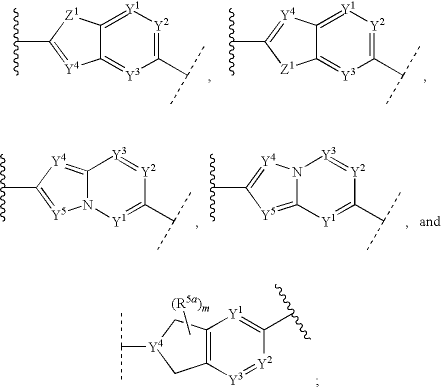

(c) a [6,5] fused bicyclic ring selected from the group consisting of:

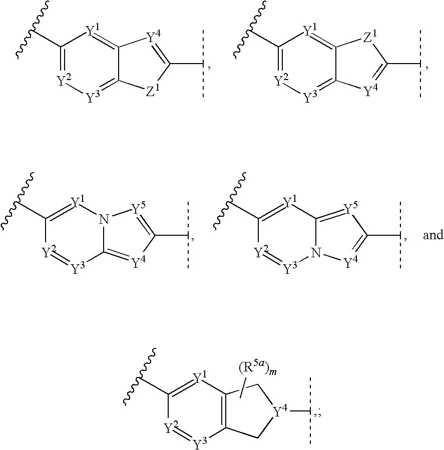

and
(d) a [5,5] fused bicyclic ring selected from the group consisting of:

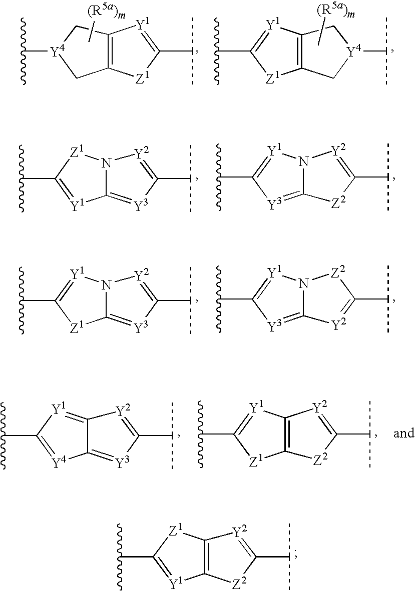

wherein the wavy line represents point of connection with X and dashed line represents point of connection with —NR¹R²;

R¹ and R² are independently selected from the group consisting of hydrogen, alkyl, substituted alkyl, cycloalkyl, substituted cycloalkyl, cycloalkenyl, substituted cycloalkenyl, aryl, substituted aryl, heterocyclyl, substituted heterocyclyl, heteroaryl, substituted heteroaryl, acyl, and aminocarbonyl, or R¹ and R² are taken together to form a group selected from heterocyclyl, substituted heterocyclyl, heteroaryl, and substituted heteroaryl; provided R¹ and R² are not both hydrogen;

Y¹, Y², Y³, Y⁴, Y⁵, and Y⁶ are independently selected from the group consisting of C—R⁵ and N; where each R⁵ is independently hydrogen or R^{5a};

R^{5a} is independently selected from the group consisting of alkyl, substituted alkyl, alkoxy, substituted alkoxy, amino, substituted amino, and halo, or optionally when m is at least 2, two R^{5a} together with the carbon atom to which they are both attached from a C═O or C═S group;

m is 0, 1, 2, 3, 4, or 5;

Z¹ and Z² are independently selected from the group consisting of C(—R⁵)₂, O, N—R⁶, S, and S(O); where each R⁶ is independently selected from the group consisting of hydrogen, alkyl, and substituted alkyl; and

X is selected from the group consisting of O, S, S(O), S(O)₂, and N—R⁴, wherein R⁴ is hydrogen, alkyl, or substituted alkyl; provided that when X is O, HET¹ is not

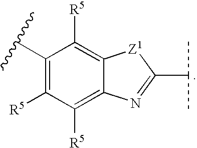

In some embodiments, the CSF-1R mediated disorder is selected from the group consisting of osteoporosis, arthritis, atherosclerosis and chronic glomerular nephritis. In some embodiments, the CSF-1R mediated disorder is rheumatoid arthritis.

In some embodiments, the CSF-1R mediated disorder is a neoplastic disease and which is not mediated by Raf kinase. In some embodiments, the neoplastic disease is a cancer selected from the group consisting of myelocytic leukemia, idiopathic myelofibrosis, breast cancer, cervical cancer, ovarian cancer, endometrial cancer, prostate cancer, hepatocellular cancer, multiple myeloma, lung cancer, renal cancer, and bone cancer.

In other embodiments of its method aspect, provided is a method for treating CSF-1R related disorders in a human or animal subject in need of such treatment comprising administering to said subject an amount of a compound of any one of Formulas (I), (II), (III), (IV), (V), (VI), and (VII), or of any one of Tables 1-4, effective to reduce or prevent tumor growth in the subject.

In other embodiments, provided is a method for treating CSF-1R related disorders in a human or animal subject in need of such treatment comprising administering to said subject an amount of a compound of any one of Formulas (I), (II), (III), (IV), (V), (VI), and (VII), or of any one of Tables 1-4, effective to reduce or prevent osteoclastogenesis, bone resorption and/or bone lesions in the subject.

In yet other embodiments, provided is a method for treating CSF-1R related disorders in a human or animal subject in need of such treatment comprising administering to said subject an amount of a compound of any one of Formulas (I), (II), (III), (IV), (V), (VI), and (VII), or of any one of Tables 1-4, effective to treat the disorder in the subject in combination with at least one additional agent for the treatment of tumor growth and/or metastasis, osteoclastogenesis, bone resorption and/or bone lesions. In a more particular embodiment the additional agent is a bisphosphonate.

In yet other embodiments, provided is a compound of any one of Formulas (I), (II), (III), (IV), (V), (VI), and (VII), or of any one of Tables 1-4 capable of selectively or preferentially inhibiting CSF-1R. In one embodiment the selective inhibitors of CSF-1R are capable of inhibiting CSF-1R at greater than about 5-fold, or about 10 fold, or about 20 fold, or about 30 fold, or about 50 fold, or about 100 fold, or about 250 fold, or about 500 fold, or about 750 fold, or about 1,000 fold, or about 2,000 fold the inhibitory activity (with respect to IC₅₀ values, for example) in Raf kinase.

In other embodiments provided is a method of inhibiting CSF-1R comprising contacting a cell with a CSF-1R inhibitor of any one of Formulas (I), (II), (III), (IV), (V), (VI), and (VII), or of any one of Tables 1-4.

In some embodiments, the inhibitory effect of CSF-1R inhibitory compounds on Raf is determined using the following biotinylated assay. The Raf kinase activity is measured by providing ATP, a recombinant kinase inactive MEK substrate and assaying the transfer of phosphate moiety to the MEK residue. Recombinant full length MEK with an inactivating K97R ATP binding site mutation (rendering kinase inactive) is expressed in E. coli and labelled with biotin post purification. The MEK cDNA is subcloned with an N-terminal (His)₆ tag and expressed in E. coli and the recombinant MEK substrate is purified from E. coli lysate by nickel affinity chromatography followed by anion exchange. The final MEK substrate preparation is biotinylated (Pierce EZ-Link Sulfo-NHS-LC-Biotin) and concentrated to about 11.25 μM. Recombinant Raf (including c-Raf and mutant B-Raf isoforms) is obtained by purification from sf9 insect cells infected with the corresponding human Raf recombinant expression vectors. The recombinant Raf isoforms are purified via a Glu antibody interaction or by Metal Ion Chromatography.

For each assay, the compound is serially diluted, for instance, starting at 25 μM with 3-fold dilutions, in DMSO and then mixed with various Raf isoforms (about 0.50 nM each). The kinase inactive biotin-MEK substrate (50 nM) is added in reaction buffer plus ATP (1 μM). The reaction buffer contains 30 mM Tris-HCl₂ pH 7.5, 10 mM MgCl₂ 2 mM DTT, 4 mM EDTA, 25 mM beta-glycerophosphate, 5 mM MnCl₂, and 0.01% BSA/PBS. Reactions are subsequently incubated for about 2 hours at room temperature and stopped by the addition of 0.5 M EDTA. Stopped reaction mixture is transferred to a neutradavin-coated plate and incubated for about 1 hour. Phosphorylated product is measured with the DELFIA time-resolved fluorescence system, using a rabbit anti-p-MEK (Cell Signaling) as the primary antibody and europium labeled anti-rabbit as the secondary antibody. Time resolved fluorescence can be read on a Wallac 1232 DELFIA fluorometer. The concentration of the compound for 50% inhibition (IC₅₀) is calculated by non-linear regression using XL Fit data analysis software.

In yet other aspects, provided is a method for treating CSF-1R related disorders in a human or animal subject in need of such treatment comprising administering to said subject an amount of a compound of any one of Formulas (I), (II), (III), (IV), (V), (VI) and (VII), or of any one of Tables 1-4 effective to reduce or prevent tumor growth in the subject in combination with at least one additional agent for the treatment of cancer. In a more particular embodiment the additional agent is a bisphosphonate.

A number of suitable anticancer agents to be used as combination therapeutics are contemplated for use. Examples of the additional anticancer agents include, but are not limited to, agents that induce apoptosis; polynucleotides (e.g., ribozymes); polypeptides (e.g., enzymes); drugs; biological mimetics; alkaloids; alkylating agents; antitumor antibiotics; antimetabolites; hormones; platinum compounds; monoclonal antibodies conjugated with anticancer drugs, toxins, and/or radionuclides; biological response modifiers (e.g. interferons [e.g. IFN-α, etc.] and interleukins [e.g. IL-2, etc.], etc.); adoptive immunotherapy agents; hematopoietic growth factors; agents that induce tumor cell differentiation (e.g. all-trans-retinoic acid, etc.); gene therapy reagents; antisense therapy reagents and nucleotides; tumor vaccines; inhibitors of angiogenesis, and the like. Numerous other examples of chemotherapeutic compounds and anticancer therapies suitable for coadministration with the disclosed compounds of any one of Formulas (I), (II), (III), (IV), (V), and (VI), or of any one of Tables 1-4 are known to those skilled in the art.

In some embodiments, additional anticancer agents to be used in combination with the compounds comprise agents that induce or stimulate apoptosis. Agents that induce apoptosis include, but are not limited to, radiation (e.g., ω); kinase inhibitors (e.g., Epidermal Growth Factor Receptor [EGFR] kinase inhibitor, Vascular Endothelial Growth Factor Receptor [VEGFR] kinase inhibitor, Fibroblast Growth Factor Receptor [FGFR] kinase inhibitor, Platelet-derived Growth Factor Receptor [PDGFR] I kinase inhibitor, and Bcr-Abl kinase inhibitors such as STI-571, Gleevec, and Glivec]); antisense molecules; antibodies [e.g., Herceptin and Rituxan]; anti-estrogens [e.g., raloxifene and tamoxifen]; anti-androgens [e.g., flutamide, bicalutamide, finasteride, aminoglutethamide, ketoconazole, and corticosteroids]; cyclooxygenase 2 (COX-2) inhibitors [e.g., Celecoxib, meloxicam, NS-398, and non-steroidal antiinflammatory drugs (NSAIDs)]; and cancer chemotherapeutic drugs [e.g., irinotecan (Camptosar), CPT-11, fludarabine (Fludara), dacarbazine (DTIC), dexamethasone, mitoxantrone, Mylotarg, VP-16, cisplatinum, 5-FU, Doxrubicin, Taxotere or taxol; cellular signaling molecules; ceramides and cytokines; and staurosprine, and the like.

The compounds of the disclosed embodiments presented herein are useful in vitro or in vivo in inhibiting the growth of cancer cells. The compounds may be used alone or in compositions together with a pharmaceutically acceptable carrier or excipient.

In other aspects, provided are pharmaceutical compositions comprising at least one compound any one of Formulas (I), (II), (III), (IV), (V), (VI), and (VII), or of any one of Tables 1-4 together with a pharmaceutically acceptable carrier suitable for administration to a human or animal subject, either alone or together with other anticancer agents.

In other aspects, provided are methods of manufacture of compounds any one of Formulas (I), (II), (III), (IV), (V), (VI), and (VII), or of any one of Tables 1-4 as described herein.

Other aspects provide pharmaceutical compositions comprising compounds of any one of Formulas (I), (II), (III), (IV), (V), (VI), and (VII), or of any one of Tables 1-4 as described herein, wherein said compound preferentially inhibits CSF-1R over Raf kinase. More particularly said compound inhibits Raf kinase at greater than about 1 μM.

Other aspects further comprise an additional agent. More particularly, said additional agent is a bisphosphonate.

Other aspects provide compounds of any one of Formulas (I), (II), (III), (IV), (V), (VI), and (VII), or of any one of Tables 1-4 effective to inhibit CSF-1R activity in a human or animal subject when administered thereto. More particularly, said compound exhibits an IC₅₀ value with respect to CSF-1R inhibition of less than about 1 μM. More particularly, said compound exhibits an IC₅₀ value with respect to Raf inhibition of greater than about 1 μM.

Another embodiment provides a method of inhibiting CSF-1R, wherein said compound selectively inhibits CSF-1R.

The compounds of the embodiments are useful in vitro or in vivo in inhibiting the growth of cancer cells. The compounds may be used alone or in compositions together with a pharmaceutically acceptable carrier or excipient.

### Administration and Pharmaceutical Composition

In general, the compounds of the embodiments will be administered in a therapeutically effective amount by any of the accepted modes of administration for agents that serve similar utilities. The actual amount of the compound, i.e., the active ingredient, will depend upon numerous factors such as the severity of the disease to be treated, the age and relative health of the subject, the potency of the compound used, the route and form of administration, and other factors. The drug can be administered more than once a day, preferably once or twice a day. All of these factors are within the skill of the attending clinician.

Effective amounts of the compounds generally include any amount sufficient to detectably inhibit CSF-1R activity by any of the assays described herein, by other CSF-1R kinase activity assays known to those having ordinary skill in the art or by detecting an inhibition or alleviation of symptoms of cancer.

The amount of active ingredient that may be combined with the carrier materials to produce a single dosage form will vary depending upon the host treated and the particular mode of administration. It will be understood, however, that the specific dose level for any particular patient will depend upon a variety of factors including the activity of the specific compound employed, the age, body weight, general health, sex, diet, time of administration, route of administration, rate of excretion, drug combination, and the severity of the particular disease undergoing therapy. The therapeutically effective amount for a given situation can be readily determined by routine experimentation and is within the skill and judgment of the ordinary clinician.

A therapeutically effective dose generally can be a total daily dose administered to a host in single or divided doses may be in amounts, for example, of from about 0.001 to about 1000 mg/kg body weight daily and from about 1.0 to about 30 mg/kg body weight daily. Dosage unit compositions may contain such amounts of submultiples thereof to make up the daily dose.

The choice of formulation depends on various factors such as the mode of drug administration and bioavailability of the drug substance. The drug can be administered as pharmaceutical compositions by any one of the following routes: oral, systemic (e.g., transdermal, intranasal or by suppository), or parenteral (e.g., intramuscular, intravenous or subcutaneous) administration. One manner of administration is oral using a convenient daily dosage regimen that can be adjusted according to the degree of affliction. Compositions can take the form of tablets, pills, capsules, semisolids, powders, sustained release formulations, solutions, suspensions, elixirs, aerosols, or any other appropriate compositions. Another manner for administion is inhalation such as for delivering a therapeutic agent directly to the respiratory tract (see U.S. Pat. No. 5,607,915).

Suitable pharmaceutically acceptable carriers or excipients include, for example, processing agents and drug delivery modifiers and enhancers, such as, for example, calcium phosphate, magnesium stearate, talc, monosaccharides, disaccharides, starch, gelatin, cellulose, methyl cellulose, sodium carboxymethyl cellulose, dextrose, hydroxypropyl-β-cyclodextrin, polyvinylpyrrolidinone, low melting waxes, ion exchange resins, and the like, as well as combinations of any two or more thereof. Liquid and semisolid excipients can be selected from glycerol, propylene glycol, water, ethanol and various oils, including those of petroleum, animal, vegetable or synthetic origin, e.g., peanut oil, soybean oil, mineral oil, sesame oil, etc. In some embodiments liquid carriers, particularly for injectable solutions, include water, saline, aqueous dextrose, and glycols. Other suitable pharmaceutically acceptable excipients are described in “Remington's Pharmaceutical Sciences,” Mack Pub. Co., New Jersey (1991).

As used herein, the term “pharmaceutically acceptable salts” refers to the nontoxic acid or alkaline earth metal salts of the compounds of any one of Formulas (I), (II), (III), (IV), (V), (VI), and (VII), or of any one of Tables 1-4. These salts can be prepared in situ during the final isolation and purification of the compounds of any one of Formulas (I), (II), (III), (IV), (V), (VI), and (VII), or of any one of Tables 1-4, or by separately reacting the base or acid functions with a suitable organic or inorganic acid or base, respectively. Representative salts include, but are not limited to, the following: acetate, adipate, alginate, citrate, aspartate, benzoate, benzenesulfonate, bisulfate, butyrate, camphorate, camphorsulfonate, digluconate, cyclopentanepropionate, dodecylsulfate, ethanesulfonate, glucoheptanoate, glycerophosphate, hemisulfate, heptanoate, hexanoate, fumarate, hydrochloride, hydrobromide, hydroiodide, 2-hydroxyethanesulfonate, lactate, maleate, methanesulfonate, nicotinate, 2-naphthalenesulfonate, oxalate, pamoate, pectinate, persulfate, 3-phenylproionate, picrate, pivalate, propionate, succinate, sulfate, tartrate, thiocyanate, p-toluenesulfonate and undecanoate. Also, the basic nitrogen-containing groups can be quaternized with agents such as alkyl halides, such as methyl, ethyl, propyl, and butyl chloride, bromides, and iodides; dialkyl sulfates like dimethyl, diethyl, dibutyl, and diamyl sulfates, long chain halides such as decyl, lauryl, myristyl and stearyl chlorides, bromides and iodides, aralkyl halides like benzyl and phenethyl bromides, and others. Water or oil-soluble or dispersible products are thereby obtained.

Examples of acids which may be employed to form pharmaceutically acceptable acid addition salts include such inorganic acids as hydrochloric acid, sulfuric acid and phosphoric acid and such organic acids as oxalic acid, maleic acid, methanesulfonic acid, succinic acid and citric acid. Basic addition salts can be prepared in situ during the final isolation and purification of the compounds of any one of Formulas (I), (II), (III), (IV), (V), (VI), and (VII), or of any one of Tables 1-4, or separately by reacting carboxylic acid moieties with a suitable base such as the hydroxide, carbonate or bicarbonate of a pharmaceutically acceptable metal cation or with ammonia, or an organic primary, secondary or tertiary amine. Pharmaceutically acceptable salts include, but are not limited to, cations based on the alkali and alkaline earth metals, such as sodium, lithium, potassium, calcium, magnesium, aluminum salts and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. Other representative organic amines useful for the formation of base addition salts include diethylamine, ethylenediamine, ethanolamine, diethanolamine, piperazine and the like.

As used herein, the term “pharmaceutically acceptable ester” refers to esters, which hydrolyze in vivo and include those that break down readily in the human body to leave the parent compound or a salt thereof. Suitable ester groups include, for example, those derived from pharmaceutically acceptable aliphatic carboxylic acids, particularly alkanoic, alkenoic, cycloalkanoic and alkanedioic acids, in which each alkyl or alkenyl moiety advantageously has not more than 6 carbon atoms. Examples of particular esters include formates, acetates, propionates, butyrates, acrylates and ethylsuccinates.

The term “pharmaceutically acceptable prodrugs” as used herein refers to those prodrugs of the compounds which are, within the scope of sound medical judgment, suitable for use in contact with the tissues of humans and lower animals without undue toxicity, irritation, allergic response, and the like, commensurate with a reasonable benefit/risk ratio, and effective for their intended use, as well as the zwitterionic forms, where possible, of the compounds of the embodiments. The term “prodrug” refers to compounds that are rapidly transformed in vivo to yield the parent compound of the above formula, for example by hydrolysis in blood. A thorough discussion is provided in T. Higuchi and V. Stella, Pro-drugs as Novel Delivery Systems, Vol. 14 of the A.C.S. Symposium Series, and in Edward B. Roche, ed., Bioreversible Carriers in Drug Design, American Pharmaceutical Association and Pergamon Press, 1987, both of which are incorporated herein by reference.

It will be apparent to those skilled in the art that the compounds of any one of Formulas (I), (II), (III), (IV), (V), (VI), and (VII), or of any one of Tables 1-4, or the pharmaceutically acceptable salts, esters, oxides, and prodrugs of any of them, may be processed in vivo through metabolism in a human or animal body or cell to produce metabolites. The term “metabolite” as used herein refers to the formula of any derivative produced in a subject after administration of a parent compound. The derivatives may be produced from the parent compound by various biochemical transformations in the subject such as, for example, oxidation, reduction, hydrolysis, or conjugation and include, for example, oxides and demethylated derivatives. The metabolites of a compound of the embodiments may be identified using routine techniques known in the art. See, e.g., Bertolini, G. et al., J. Med. Chem. 40:2011-2016 (1997); Shan, D. et al., J. Pharm. Sci. 86(7):765-767; Bagshawe K., Drug Dev. Res. 34:220-230 (1995); Bodor, N., Advances in Drug Res. 13:224-331 (1984); Bundgaard, H., Design of Prodrugs (Elsevier Press 1985); and Larsen, I. K., Design and Application of Prodrugs, Drug Design and Development (Krogsgaard-Larsen et al., eds., Harwood Academic Publishers, 1991). It should be understood that individual chemical compounds that are metabolites of the compounds of any one of Formulas (I), (II), (III), (IV), (V), (VI), and (VII), or of any one of Tables 1-4, or the pharmaceutically acceptable salts, esters, oxides and prodrugs of any of them, are included within the embodiments provided herein.

The compounds of the preferred embodiments may be administered orally, parenterally, sublingually, by aerosolization or inhalation spray, rectally, or topically in dosage unit formulations containing conventional nontoxic pharmaceutically acceptable carriers, adjuvants, and vehicles as desired. Topical administration may also involve the use of transdermal administration such as transdermal patches or ionophoresis devices. The term parenteral as used herein includes subcutaneous injections, intravenous, intrathecal, intramuscular, intrasternal injection, or infusion techniques.

Injectable preparations, for example, sterile injectable aqueous or oleaginous suspensions may be formulated according to the known art using suitable dispersing or wetting agents and suspending agents. The sterile injectable preparation may also be a sterile injectable solution or suspension in a nontoxic parenterally acceptable diluent or solvent, for example, as a solution in 1,3-propanediol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed including synthetic mono- or di-glycerides. In addition, fatty acids such as oleic acid find use in the preparation of injectables.

Suppositories for rectal administration of the drug can be prepared by mixing the drug with a suitable nonirritating excipient such as cocoa butter and polyethylene glycols, which are solid at ordinary temperatures but liquid at the rectal temperature and will therefore melt in the rectum and release the drug.

Solid dosage forms for oral administration may include capsules, tablets, pills, powders, and granules. In such solid dosage forms, the active compound may be admixed with at least one inert diluent such as sucrose lactose or starch. Such dosage forms may also comprise, as is normal practice, additional substances other than inert diluents, e.g., lubricating agents such as magnesium stearate. In the case of capsules, tablets, and pills, the dosage forms may also comprise buffering agents. Tablets and pills can additionally be prepared with enteric coatings.

Liquid dosage forms for oral administration may include pharmaceutically acceptable emulsions, solutions, suspensions, syrups, and elixirs containing inert diluents commonly used in the art, such as water. Such compositions may also comprise adjuvants, such as wetting agents, emulsifying and suspending agents, cyclodextrins, and sweetening, flavoring, and perfuming agents.

The compounds of the embodiments can also be administered in the form of liposomes. As is known in the art, liposomes are generally derived from phospholipids or other lipid substances. Liposomes are formed by mono- or multi-lamellar hydrated liquid crystals that are dispersed in an aqueous medium. Any non-toxic, physiologically acceptable and metabolizable lipid capable of forming liposomes can be used. The present compositions in liposome form can contain stabilizers, preservatives, excipients, and the like. Examples of lipids are the phospholipids and phosphatidyl cholines (lecithins), both natural and synthetic. Methods to form liposomes are known in the art. See, for example, Prescott, Ed., Methods in Cell Biology, Volume XIV, Academic Press, New York, N.W., p. 33 et seq. (1976).

Compressed gases may be used to disperse a compound of the embodiments in aerosol form. Inert gases suitable for this purpose are nitrogen, carbon dioxide, etc. Other suitable pharmaceutical excipients and their formulations are described in Remington's Pharmaceutical Sciences, edited by E. W. Martin (Mack Publishing Company, 18th ed., 1990).

For delivery via inhalation the compound can be formulated as liquid solution, suspensions, aerosol propellants or dry powder and loaded into a suitable dispenser for administration. There are several types of pharmaceutical inhalation devices-nebulizer inhalers, metered dose inhalers (MDI) and dry powder inhalers (DPI). Nebulizer devices produce a stream of high velocity air that causes the therapeutic agents (which are formulated in a liquid form) to spray as a mist that is carried into the patient's respiratory tract. MDI's typically are formulation packaged with a compressed gas. Upon actuation, the device discharges a measured amount of therapeutic agent by compressed gas, thus affording a reliable method of administering a set amount of agent. DPI dispenses therapeutic agents in the form of a free flowing powder that can be dispersed in the patient's inspiratory air-stream during breathing by the device. In order to achieve a free flowing powder, the therapeutic agent is formulated with an excipient such as lactose. A measured amount of the therapeutic agent is stored in a capsule form and is dispensed with each actuation.

Recently, pharmaceutical formulations have been developed especially for drugs that show poor bioavailability based upon the principle that bioavailability can be increased by increasing the surface area i.e., decreasing particle size. For example, U.S. Pat. No. 4,107,288 describes a pharmaceutical formulation having particles in the size range from about 10 to about 1,000 nm in which the active material is supported on a crosslinked matrix of macromolecules. U.S. Pat. No. 5,145,684 describes the production of a pharmaceutical formulation in which the drug substance is pulverized to nanoparticles (average particle size of about 400 nm) in the presence of a surface modifier and then dispersed in a liquid medium to give a pharmaceutical formulation that exhibits remarkably high bioavailability.

### Combination Therapies

While the compounds of the embodiments can be administered as the sole active pharmaceutical agent, they can also be used in combination with one or more other agents used in the treatment of cancer. The compounds of the embodiments are also useful in combination with known therapeutic agents and anti-cancer agents, and combinations of the presently disclosed compounds with other anti-cancer or chemotherapeutic agents are within the scope of the embodiments. Examples of such agents can be found in Cancer Principles and Practice of Oncology, V. T. Devita and S. Hellman (editors), 6th edition (Feb. 15, 2001), Lippincott Williams & Wilkins Publishers. A person of ordinary skill in the art would be able to discern which combinations of agents would be useful based on the particular characteristics of the drugs and the cancer involved. Such anti-cancer agents include, but are not limited to, the following: estrogen receptor modulators, androgen receptor modulators, retinoid receptor modulators, cytotoxic/cytostatic agents, antiproliferative agents, prenyl-protein transferase inhibitors, HMG-CoA reductase inhibitors and other angiogenesis inhibitors, inhibitors of cell proliferation and survival signaling, apoptosis inducing agents and agents that interfere with cell cycle checkpoints. The compounds of the embodiments are also useful when co-administered with radiation therapy.

Therefore, in one embodiment, the compounds are also used in combination with known anticancer agents including, for example, estrogen receptor modulators, androgen receptor modulators, retinoid receptor modulators, cytotoxic agents, antiproliferative agents, prenyl-protein transferase inhibitors, HMG-CoA reductase inhibitors, HIV protease inhibitors, reverse transcriptase inhibitors, and other angiogenesis inhibitors.

Estrogen receptor modulators are compounds that can interfere with or inhibit the binding of estrogen to the receptor, regardless of mechanism. Examples of estrogen receptor modulators include, but are not limited to, tamoxifen, raloxifene, idoxifene, LY353381, LY117081, toremifene, fulvestrant, 4-[7-(2,2-dimethyl-1-oxopropoxy-4-methyl-2-[4-[2-(1-piperidinyl)ethoxy]phenyl]-2H-1-benzopyran-3-yl]-phenyl-2,2-di-methylpropanoate, 4,4′-dihydroxybenzophenone-2,4-dinitrophenyl-hydrazone, and SH646.

Androgen receptor modulators are compounds which can interfere with or inhibit the binding of androgens to an androgen receptor. Representative examples of androgen receptor modulators include finasteride and other 5α-reductase inhibitors, nilutamide, flutamide, bicalutamide, liarozole, and abiraterone acetate. Retinoid receptor modulators are compounds which interfere or inhibit the binding of retinoids to a retinoid receptor. Examples of retinoid receptor modulators include bexarotene, tretinoin, 13-cis-retinoic acid, 9-cis-retinoic acid, α-difluoromethylornithine, LX23-7553, trans-N-(4′-hydroxyphenyl)retinamide, and N4-carboxyphenyl retinamide.

Cytotoxic and/or cytostatic agents are compounds which can cause cell death or inhibit cell proliferation primarily by interfering directly with the cell's functioning or inhibit or interfere with cell mytosis, including alkylating agents, tumor necrosis factors, intercalators, hypoxia activatable compounds, microtubule inhibitors/microtubule-stabilizing agents, inhibitors of mitotic kinesins, inhibitors of kinases involved in mitotic progression, antimetabolites; biological response modifiers; hormonal/anti-hormonal therapeutic agents, haematopoietic growth factors, monoclonal antibody targeted therapeutic agents, topoisomerase inhibitors, proteasome inhibitors and ubiquitin ligase inhibitors. Examples of cytotoxic agents include, but are not limited to, sertenef, cachectin, ifosfamide, tasonermin, lonidamine, carboplatin, altretamine, prednimustine, dibromodulcitol, ranimustine, fotemustine, nedaplatin, oxaliplatin, temozolomide, heptaplatin, estramustine, improsulfan tosilate, trofosfamide, nimustine, dibrospidium chloride, pumitepa, lobaplatin, satraplatin, profiromycin, cisplatin, irofulven, dexifosfamide, cis-aminedichloro(2-methyl-pyridine)platinum, benzylguanine, glufosfamide, GPX100, (trans, trans, trans)-bis-mu-(hexane-1,6-diamine)-mu-[diamine-platinum(II)]bis[diamine(chloro)platinum (II)]tetrachloride, diarizidinylspermine, arsenic trioxide, 1-(11-dodecylamino-10-hydroxyundecyl)-3,7-dimethylxanthine, zorubicin, idarubicin, daunorubicin, bisantrene, mitoxantrone, pirarubicin, pinafide, valrubicin, amrubicin, antineoplaston, 3′-deamino-3′-morpholino-13-deoxo-10-hydroxycamino-mycin, annamycin, galarubicin, elinafide, MEN10755, and 4-demethoxy-3-deamino-3-aziridinyl-4-methylsulphonyl-daunorubicin (see WO 00/50032). A representative example of a hypoxia activatable compound is tirapazamine. Proteasome inhibitors include, but are not limited to, lactacystin and bortezomib. Examples of microtubule inhibitors/microtubule-stabilizing agents include paclitaxel, vindesine sulfate, 3′,4′-didehydro-4′-deoxy-8′-norvincaleukoblastine, docetaxol, rhizoxin, dolastatin, mivobulin isethionate, auristatin, cemadotin, RPR109881, BMS184476, vinflunine, cryptophycin, 2,3,4,5,6-pentafluoro-N-(3-fluoro-4-methoxyphenyl)benzene sulfonamide, anhydro-vinblastine, N,N-dimethyl-L-valyl-L-valyl-N-methyl-L-valyl-L-prolyl-L-proline-t-butyl-amide, TDX258, the epothilones (see for example U.S. Pat. Nos. 6,284,781 and 6,288,237) and BMS188797. Representative examples of topoisomerase inhibitors include topotecan, hycaptamine, irinotecan, rubitecan, 6-ethoxypropionyl-3′,4′-O-exo-benzylidene-chartreusin, 9-methoxy-N,N-dimethyl-5-nitropyrazolo[3,4,5-kl]acridine-2-(6H) propanamine, 1-amino-9-ethyl-5-fluoro-2,3-dihydro-9-hydroxy-4-methyl-1H,12H-benzo[de]pyrano[3′,4′:b,7]-indolizino[1,2b]quinoline-10,13(9H,15H)dione, lurtotecan, 7-[2-(N-isopropylamino)ethyl]-(20S)camptothecin, BNP1350, BNPI1100, BN80915, BN80942, etoposide phosphate, teniposide, sobuzoxane, 2′-dimethylamino-2′-deoxy-etoposide, GL331, N-[2-(dimethylamino)ethyl]-9-hydroxy-5,6-dimethyl-6H-pyrido[4,3-b]carbazole-1-carboxamide, asulacrine, (5a,5aB,8aa,9b)-9-[2-[N42-(dimethylamino)-ethyl]-N-methylamino]ethyl]-5-[4-hydroOxy-3,5-dimethoxyphenyl]-5,5a,6,8,8a,9-hexa-hydrofuro(3′,4′:6,7)naphtho(2,3-d)-1,3-dioxol-6-one, 2,3-(methylenedioxy)-5-methyl-7-hydroxy-8-methoxybenzo[c]-phenanthridinium, 6,9-bis[(2-aminoethyl)amino]benzo-[g]isoquinoline-5,10-dione, 5-(3-aminopropylamino)-7,10-dihydroxy-2-(2-hydroxyethyl-aminomethyl)-6H-pyrazolo[4,5,1′-de]acridin-6-one, N-[1-[2(diethylamino)ethylamino]-7-methoxy-9-oxo-9H-thioxanthen-4-ylmethyl]formamide, N-(2-(dimethylamino)ethyl)-acridine-4-carboxamide, 6-[[2-(dimethylamino)ethyl]amino]-3-hydroxy-7H-indeno[2,1-c]quinolin-7-one, and dimesna. Examples of inhibitors of mitotic kinesins, such as the human mitotic kinesin KSP, are described in PCT Publications WO 01/30768 and WO 01/98278, WO 03/050,064 (Jun. 19, 2003), WO 03/050,122 (Jun. 19, 2003), WO 03/049,527 (Jun. 19, 2003), WO 03/049,679 (Jun. 19, 2003), WO 03/049,678 (Jun. 19, 2003) and WO 03/39460 (May 15, 2003) and pending PCT Appl. Nos. US03/06403 (filed Mar. 4, 2003), US03/15861 (filed May 19, 2003), US03/15810 (filed May 19, 2003), US03/18482 (filed Jun. 12, 2003) and US03/18694 (filed Jun. 12, 2003). In an embodiment inhibitors of mitotic kinesins include, but are not limited to inhibitors of KSP, inhibitors of MKLP1, inhibitors of CENP-E, inhibitors of MCAK, inhibitors of Kif14, inhibitors of Mphosph1 and inhibitors of Rab6-KIFL.

Inhibitors of kinases involved in mitotic progression include, but are not limited to, inhibitors of aurora kinase, inhibitors of Polo-like kinases (PLK) (e.g., inhibitors of PLK-1), inhibitors of bub-1 and inhibitors of bub-1R. Antiproliferative agents include antisense RNA and DNA oligonucleotides such as G3139, ODN698, RVASKRAS, GEM231, and INX3001, and antimetabolites such as enocitabine, carmofur, tegafur, pentostatin, doxifluridine, trimetrexate, fludarabine, capecitabine, galocitabine, cytarabine ocfosfate, fosteabine sodium hydrate, raltitrexed, paltitrexid, emitefur, tiazofurin, decitabine, nolatrexed, pemetrexed, nelzarabine, 2′-deoxy-2′-methylidene-cytidine, 2′-fluoromethylene-2′-deoxycytidine, N-[5-(2,3-dihydro-benzofuryl)sulfonyl]-N′-(3,4-dichlorophenyl)urea, N6-[4-deoxy-4-[N2-[2(E),4(E)-tetradecadienoyl]glycyl-amino]-L-glycero-B-L-manno-heptopyranosyl]adenine, aplidine, ecteinascidin, troxa-citabine, 4-[2-amino-4-oxo-4,6,7,8-tetrahydro-3H-pyrimidino[5,4-b][1,4]thiazin-6-yl-(S)-ethyl]-2,5-thienoyl-L-glutamic acid, aminopterin, 5-fluorouracil, alanosine, 11-acetyl-8-(carbamoyloxymethyl)-4-formyl-6-methoxy-14-oxa-1,1-diazatetracyclo(7.4.1.0.0)-tetra-deca-2,4,6-trien-9-yl acetic acid ester, swainsonine, lometrexol, dexrazoxane, methioninase, 2′-cyano-2′-deoxy-N4-palmitoyl-1-B-D-arabino furanosyl cytosine and 3-aminopyridine-2-carboxaldehyde thiosemicarbazone. Examples of monoclonal antibody targeted therapeutic agents include those therapeutic agents which have cytotoxic agents or radioisotopes attached to a cancer cell specific or target cell specific monoclonal antibody. Examples include, for example, Bexxar. HMG-CoA reductase inhibitors are inhibitors of 3-hydroxy-3-methylglutaryl-CoA reductase. Compounds which have inhibitory activity for HMG-CoA reductase can be readily identified by using assays well-known in the art such as those described or cited in U.S. Pat. No. 4,231,938 and WO 84/02131. Examples of HMG-CoA reductase inhibitors that may be used include, but are not limited to, lovastatin (MEVACOR®; see U.S. Pat. Nos. 4,231,938, 4,294,926 and 4,319,039), simvastatin (ZOCOR®; see U.S. Pat. Nos. 4,444,784, 4,820,850 and 4,916,239), pravastatin (PRAVACHOL®; see U.S. Pat. Nos. 4,346,227, 4,537,859, 4,410,629, 5,030,447 and 5,180,589), fluvastatin (LESCOL®; see U.S. Pat. Nos. 5,354,772, 4,911,165, 4,929,437, 5,189,164, 5,118,853, 5,290,946 and 5,356,896) and atorvastatin (LIPITOR®; see U.S. Pat. Nos. 5,273,995, 4,681,893, 5,489,691 and 5,342,952). The structural formulas of these and additional HMG-CoA reductase inhibitors that may be used in the instant methods are described at page 87 of M. Yalpani, “Cholesterol Lowering Drugs”, Chemistry & Industry, pp. 85-89 (5 Feb. 1996) and U.S. Pat. Nos. 4,782,084 and 4,885,314. In an embodiment, the HMG-CoA reductase inhibitor is selected from lovastatin or simvastatin.

Prenyl-protein transferase inhibitors are compounds which inhibit any one or any combination of the prenyl-protein transferase enzymes, including farnesyl-protein transferase (FPTase), geranylgeranyl-protein transferase type I (GGPTase-I), and geranylgeranyl-protein transferase type-II (GGPTase-II, also called Rab GGPTase). Examples of prenyl-protein transferase inhibiting compounds include (±)-6-[amino(4-chlorophenyl)(1-methyl-1H-imidazol-5-yl)methyl]-4-(3-chlorophenyl)-1-methyl-2(1H)-quinolinone, (−)-6-[amino(4-chlorophenyl)(1-methyl-1H-imidazol-5-yl)methyl]-4-(3-chlorophenyl)-1-methyl-2(1H)-quinolinone, (+)-6-[amino(4-chlorophenyl)(1-methyl-1H-imidazol-5-yl)methyl]-4-(3-chlorophenyl)-1-methyl-2(1H)-quinolinone, 5(S)-n-butyl-1-(2,3-dimethylphenyl)-4-[1-(4-cyanobenzyl)-5-imidazolylmethyl-2-piperazinone, (S)-1-(3-chlorophenyl)-4-[1-(4-cyanobenzyl)-5-imidazolylmethyl]-5-[2-(ethanesulfonyl)methyl)-2-piperazinone, 5(S)-n-butyl-1-(2-methylphenyl)-4-[1-(4-cyanobenzyl)-5-imidazolylmethyl]-2-piperazinone, 1-(3-chlorophenyl)-4-[1-(4-cyanobenzyl)-2-methyl-5-imidazolylmethyl]-2-piperazinone, 1-(2,2-diphenylethyl)-3-[N-(1-(4-cyanobenzyl)-1H-imidazol-5-ylethyl)carbamoyl]piperidine, 4-{-[4-hydroxymethyl-4-(4-chloropyridin-2-ylmethyl)-piperidine-1-ylmethyl]-2-methylimidazol-1-ylmethyl}benzonitrile, 4-{-5-[4-hydroxymethyl-4-(3-chlorobenzyl)-piperidine-1-ylmethyl]-2-methylimidazol-1-yl-methyl}benzonitrile, 4-{3-[4-(2-oxo-2H-pyridin-1-yl)benzyl]-3H-imidazol-4-ylmethyl}-benzonitrile, 4-{3-[4-(5-chloro-2-oxo-2H-[1,2]bipyridin-5′-ylmethyl]-3H-imidazol-4-yl-methyl]benzonitrile, 4-{3-[4-(2-oxo-2H-[1,2]bipyridin-5′-ylmethyl]-3H-imidazol4-yl-methyl]benzonitrile, 4-[3-(2-oxo-1-phenyl-1,2-dihydropyridin-4-ylmethyl)-3H-imidazol-4-ylmethyl]benzonitrile, 18,19-dihydro-[9-oxo-5H,17H-6, 10:12,16-dimetheno-1H-imidazo[4,3-c][1,11,4] dioxaazacyclo-nonadecine-9-carbonitrile, (.+−.)-19,20-dihydro-[9-oxo-5H-18,21-ethano-12,14-etheno-6,10-metheno-22H-benzo[d]imidazo[4,3-k]-[1,6,9,12]oxatriaza-cyclooctadecine-9-carbonitrile, 19,20-dihydro-[9-oxo-5H,17H-18,21-ethano-6, 10:12,16-dimetheno-22H-imidazo[3,4-h][1,8,11,14]oxa-triazacycloeicosine-9-carbonitrile, and (.+−.)-19,20-dihydro-3-methyl-[9-oxo-5H-18,21-ethano-12,14-etheno-6,10-metheno-22H-benzo[d]imidazo[4,3-k][1,6,9,12]oxa-triazacyclooctadecine-9-carbonitrile. Other examples of prenyl-protein transferase inhibitors can be found in the following publications and patents: WO 96/30343, WO 97/18813, WO 97/21701, WO 97/23478, WO 97/38665, WO 98/28980, WO 98/29119, WO 95/32987, U.S. Pat. No. 5,420,245, U.S. Pat. No. 5,523,430, U.S. Pat. No. 5,532,359, U.S. Pat. No. 5,510,510, U.S. Pat. No. 5,589,485, U.S. Pat. No. 5,602,098, European Patent Publ. 0 618 221, European Patent Publ. 0 675 112, European Patent Publ. 0 604 181, European Patent Publ. 0 696 593, WO 94/19357, WO 95/08542, WO 95/11917, WO 95/12612, WO 95/12572, WO 95/10514, U.S. Pat. No. 5,661,152, WO 95/10515, WO 95/10516, WO 95/24612, WO 95/34535, WO 95/25086, WO 96/05529, WO 96/06138, WO 96/06193, WO 96/16443, WO 96/21701, WO 96/21456, WO 96/22278, WO 96/24611, WO 96/24612, WO 96/05168, WO 96/05169, WO 96/00736, U.S. Pat. No. 5,571,792, WO 96/17861, WO 96/33159, WO 96/34850, WO 96/34851, WO 96/30017, WO 96/30018, WO 96/30362, WO 96/30363, WO 96/31111, WO 96/31477, WO 96/31478, WO 96/31501, WO 97/00252, WO 97/03047, WO 97/03050, WO 97/04785, WO 97/02920, WO 97/17070, WO 97/23478, WO 97/26246, WO 97/30053, WO 97/44350, WO 98/02436, and U.S. Pat. No. 5,532,359. For an example of the role of a prenyl-protein transferase inhibitor on angiogenesis see European J. of Cancer 35(9):1394-1401 (1999).

Angiogenesis inhibitors refers to compounds that can inhibit the formation of new blood vessels, regardless of mechanism. Examples of angiogenesis inhibitors include, but are not limited to, tyrosine kinase inhibitors, such as inhibitors of the tyrosine kinase receptors Flt-1 (VEGFR1) and Flk-1/KDR (VEGFR2), inhibitors of epidermal-derived, fibroblast-derived, or platelet derived growth factors, MMP (matrix metalloprotease) inhibitors, integrin blockers, interferon-.alpha., interleukin-12, pentosan polysulfate, cyclooxygenase inhibitors, including nonsteroidal anti-inflammatories (NSAIDs) like aspirin and ibuprofen as well as selective cyclooxy-genase-2 inhibitors like celecoxib and rofecoxib (PNAS 89:7384 (1992); JNCI 69:475 (1982); Arch. Opthalmol. 108:573 (1990); Anat. Rec., (238):68 (1994); FEBS Letters 372:83 (1995); Clin, Orthop. 313:76 (1995); J. Mol. Endocrinol. 16:107 (1996); Jpn. J. Pharmacol. 75:105 (1997); Cancer Res. 57:1625 (1997); Cell 93:705 (1998); Intl. J. Mol. Med. 2:715 (1998); J. Biol. Chem. 274:9116 (1999)), steroidal anti-inflammatories (such as corticosteroids, mineralocorticoids, dexamethasone, prednisone, prednisolone, methylpred, betamethasone), carboxyamidotriazole, combretastatin A4, squalamine, 6-O-chloroacetyl-carbonylgumagillol, thalidomide, angiostatin, troponin-1, angiotensin II antagonists (see Fernandez et al., J. Lab. Clin. Med. 105:141-145 (1985)), and antibodies to VEGF (see, Nature Biotechnology, 17:963-968 (October 1999); Kim et al., Nature, 362:841-844 (1993); WO 00/44777; and WO 00/61186). Other therapeutic agents that modulate or inhibit angiogenesis and may also be used in combination with the compounds of the embodiments include agents that modulate or inhibit the coagulation and fibrinolysis systems (see review in Clin. Chem. La. Med. 38:679-692 (2000)). Examples of such agents that modulate or inhibit the coagulation and fibrinolysis pathways include, but are not limited to, heparin (see Thromb. Haemost. 80:10-23 (1998)), low molecular weight heparins and carboxypeptidase U inhibitors (also known as inhibitors of active thrombin activatable fibrinolysis inhibitor [TAFIa]) (see Thrombosis Res. 101:329-354 (2001)). TAFIa inhibitors have been described in PCT Publication WO 03/013,526 and U.S. Ser. No. 60/349,925 (filed Jan. 18, 2002). The embodiments also encompass combinations of the compounds of the embodiments with NSAIDs which are selective COX-2 inhibitors (generally defined as those which possess a specificity for inhibiting COX-2 over COX-1 of at least about 100 fold as measured by the ratio of IC₅₀ for COX-2 over IC₅₀ for COX-1 evaluated by cell or microsomal assays). Such compounds include, but are not limited to those disclosed in U.S. Pat. No. 5,474,995, issued Dec. 12, 1995, U.S. Pat. No. 5,861,419, issued Jan. 19, 1999, U.S. Pat. No. 6,001,843, issued Dec. 14, 1999, U.S. Pat. No. 6,020,343, issued Feb. 1, 2000, U.S. Pat. No. 5,409,944, issued Apr. 25, 1995, U.S. Pat. No. 5,436,265, issued Jul. 25, 1995, U.S. Pat. No. 5,536,752, issued Jul. 16, 1996, U.S. Pat. No. 5,550,142, issued Aug. 27, 1996, U.S. Pat. No. 5,604,260, issued Feb. 18, 1997, U.S. Pat. No. 5,698,584, issued Dec. 16, 1997, U.S. Pat. No. 5,710,140, issued Jan. 20, 1998, WO 94/15932, published Jul. 21, 1994, U.S. Pat. No. 5,344,991, issued Jun. 6, 1994, U.S. Pat. No. 5,134,142, issued Jul. 28, 1992, U.S. Pat. No. 5,380,738, issued Jan. 10, 1995, U.S. Pat. No. 5,393,790, issued Feb. 20, 1995, U.S. Pat. No. 5,466,823, issued Nov. 14, 1995, U.S. Pat. No. 5,633,272, issued May 27, 1997, and U.S. Pat. No. 5,932,598, issued Aug. 3, 1999, all of which are hereby incorporated by reference. Representative inhibitors of COX-2 that are useful in the methods of the embodiments include 3-phenyl-4-(4-(methylsulfonyl)phenyl)-2-(5H)-furanone; and 5-chloro-3-(4-methylsulfonyl)phenyl-2-(2-methyl-5-pyridinyl)pyridine. Compounds which are described as specific inhibitors of COX-2 and are therefore useful in the embodiments, and methods of synthesis thereof, can be found in the following patents, pending applications and publications, which are herein incorporated by reference: WO 94/15932, published Jul. 21, 1994, U.S. Pat. No. 5,344,991, issued Jun. 6, 1994, U.S. Pat. No. 5,134,142, issued Jul. 28, 1992, U.S. Pat. No. 5,380,738, issued Jan. 10, 1995, U.S. Pat. No. 5,393,790, issued Feb. 20, 1995, U.S. Pat. No. 5,466,823, issued Nov. 14, 1995, U.S. Pat. No. 5,633,272, issued May 27, 1997, U.S. Pat. No. 5,932,598, issued Aug. 3, 1999, U.S. Pat. No. 5,474,995, issued Dec. 12, 1995, U.S. Pat. No. 5,861,419, issued Jan. 19, 1999, U.S. Pat. No. 6,001,843, issued Dec. 14, 1999, U.S. Pat. No. 6,020,343, issued Feb. 1, 2000, U.S. Pat. No. 5,409,944, issued Apr. 25, 1995, U.S. Pat. No. 5,436,265, issued Jul. 25, 1995, U.S. Pat. No. 5,536,752, issued Jul. 16, 1996, U.S. Pat. No. 5,550,142, issued Aug. 27, 1996, U.S. Pat. No. 5,604,260, issued Feb. 18, 1997, U.S. Pat. No. 5,698,584, issued Dec. 16, 1997, and U.S. Pat. No. 5,710,140, issued Jan. 20, 1998. Other examples of angiogenesis inhibitors include, but are not limited to, endostatin, ukrain, ranpirnase, IM862, 5-methoxy-4-[2-methyl-3-(3-methyl-2-butenyl)oxiranyl]-1-oxaspiro[2,5]oct-6-yl(chloroacetyl)carbamate, acetyldinanaline, 5-amino-1-[[3,5-dichloro-4-(4-chlorobenzoyl)phenyl]methyl]-1H-1,2,3-triazole-4-carboxamide, CM101, squalamine, combretastatin, RPI4610, NX31838, sulfated mannopentaose phosphate, 7,7-(carbonyl-bis[imino-N-methyl-4,2-pyrrolo-carbonylimino[N-methyl-4,2-pyrrole]-carbonylimino]-bis-(1,3-naphthalene disulfonate), and 3-[(2,4-dimethylpyrrol-5-yl)methylene]-2-indolinone (SU5416).

Agents that interfere with cell cycle checkpoints are compounds that can inhibit protein kinases that transduce cell cycle checkpoint signals, thereby sensitizing the cancer cell to DNA damaging agents. Such agents include inhibitors of ATR, ATM, the Chk1 and Chk2 kinases and cdk and cdc kinase inhibitors and are specifically exemplified by 7-hydroxystaurosporin, flavopiridol, CYC202 (Cyclacel) and BMS-387032.

Inhibitors of cell proliferation and survival signaling pathway can be pharmaceutical agents that can inhibit cell surface receptors and signal transduction cascades downstream of those surface receptors. Such agents include inhibitors of inhibitors of EGFR (for example gefitinib and erlotinib), inhibitors of ERB-2 (for example trastuzumab), inhibitors of IGFR, inhibitors of cytokine receptors, inhibitors of MET, inhibitors of PI3K (for example LY294002), serine/threonine kinases (including but not limited to inhibitors of Akt such as described in WO 02/083064, WO 02/083139, WO 02/083140 and WO 02/083138), inhibitors of Raf kinase (for example BAY-43-9006), inhibitors of MEK (for example CI-1040 and PD-098059) and inhibitors of mTOR (for example Wyeth CCI-779). Such agents include small molecule inhibitor compounds and antibody antagonists.

Apoptosis inducing agents include activators of TNF receptor family members (including the TRAIL receptors).

In certain embodiments, representative agents useful in combination with the compounds of the embodiments for the treatment of cancer include, for example, irinotecan, topotecan, gemcitabine, 5-fluorouracil, leucovorin carboplatin, cisplatin, taxanes, tezacitabine, cyclophosphamide, vinca alkaloids, imatinib (Gleevec), anthracyclines, rituximab, trastuzumab, as well as other cancer chemotherapeutic agents.

The above compounds to be employed in combination with the compounds of the embodiments can be used in therapeutic amounts as indicated in the Physicians' Desk Reference (PDR) 47th Edition (1993), which is incorporated herein by reference, or such therapeutically useful amounts as would be known to one of ordinary skill in the art.

The compounds of the embodiments and the other anticancer agents can be administered at the recommended maximum clinical dosage or at lower doses. Dosage levels of the active compounds in the compositions of the embodiments may be varied so as to obtain a desired therapeutic response depending on the route of administration, severity of the disease and the response of the patient. The combination can be administered as separate compositions or as a single dosage form containing both agents. When administered as a combination, the therapeutic agents can be formulated as separate compositions, which are given at the same time or different times, or the therapeutic agents, can be given as a single composition.

### General Synthetic Methods

The compounds disclosed herein can be prepared from readily available starting materials using the following general methods and procedures. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvent used, but such conditions can be determined by one skilled in the art by routine optimization procedures.

Additionally, as will be apparent to those skilled in the art, conventional protecting groups may be necessary to prevent certain functional groups from undergoing undesired reactions. Suitable protecting groups for various functional groups as well as suitable conditions for protecting and deprotecting particular functional groups are well known in the art. For example, numerous protecting groups are described in T. W. Greene and G. M. Wuts, Protecting Groups in Organic Synthesis, Third Edition, Wiley, New York, 1999, and references cited therein.

Furthermore, the compounds disclosed herein may contain one or more chiral centers. Accordingly, if desired, such compounds can be prepared or isolated as pure stereoisomers, i.e., as individual enantiomers or diastereomers, or as stereoisomer-enriched mixtures. All such stereoisomers (and enriched mixtures) are included within the scope of the embodiments, unless otherwise indicated. Pure stereoisomers (or enriched mixtures) may be prepared using, for example, optically active starting materials or stereoselective reagents well-known in the art. Alternatively, racemic mixtures of such compounds can be separated using, for example, chiral column chromatography, chiral resolving agents and the like.

The starting materials for the following reactions are generally known compounds or can be prepared by known procedures or obvious modifications thereof. For example, many of the starting materials are available from commercial suppliers such as Aldrich Chemical Co. (Milwaukee, Wis., USA), Bachem (Torrance, Calif., USA), Emka-Chemce or Sigma (St. Louis, Mo., USA). Others may be prepared by procedures, or obvious modifications thereof, described in standard reference texts such as Fieser and Fieser's Reagents for Organic Synthesis, Volumes 1-15 (John Wiley and Sons, 1991), Rodd's Chemistry of Carbon Compounds, Volumes 1-5 and Supplementals (Elsevier Science Publishers, 1989), Organic Reactions, Volumes 1-40 (John Wiley and Sons, 1991), March's Advanced Organic Chemistry, (John Wiley and Sons, 4^{th} Edition), and Larock's Comprehensive Organic Transformations (VCH Publishers Inc., 1989).

The various starting materials, intermediates, and compounds of the embodiments may be isolated and purified where appropriate using conventional techniques such as precipitation, filtration, crystallization, evaporation, distillation, and chromatography. Characterization of these compounds may be performed using conventional methods such as by melting point, mass spectrum, nuclear magnetic resonance, and various other spectroscopic analyses.

Compounds of the embodiments may generally be prepared using a number of methods familiar to one of skill in the art, and may generally be made in accordance with the following reaction Schemes 1-6, which are described in detail in the Examples below.

### General Schemes:

Schemes 1-6 illustrate general methods for the preparation of intermediates and compounds of the embodiments. These compounds are prepared from starting materials either known in the art for commercially available. The specific compounds are for illustrative purposes only.

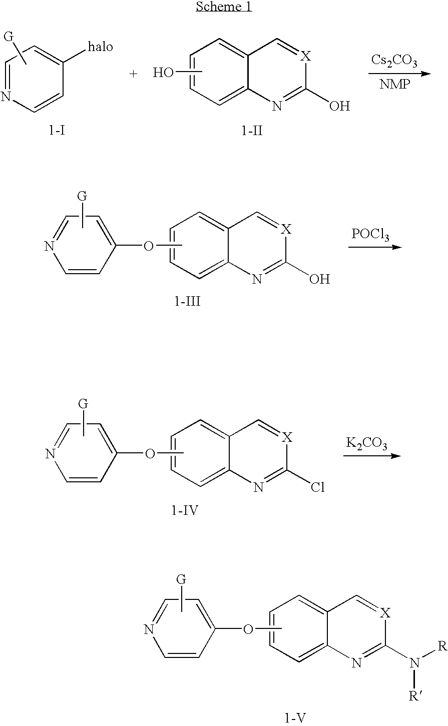

As shown in Scheme 1, compounds of the invention of formula I-V, can be prepared by methods know to those trained in the art starting from a hydroxyl-2-quinazalone (I-II, X═N), or derivative thereof, or hydroxyl-2-quinalone (I-II, X═CH), or derivative thereof, and reacting with a substituted or unsubstituted a halopyridine 1-I, or derivative thereof, to form intermediates of formula I-III. Treatment of intermediate of formula I-III, with POCl₃, or equivalent reagent, then provide the chloro quin(az)oline intermediate of formula I-IV. Subsequent treatment with an primary or secondary amine in a solvent such as, for example, DMF or NMP, and a base such as, for example, K₂CO_{3 or Cs}₂CO₃ at temperatures between room temperature to 200° C. provides compounds of the invention of general structure 1-V. It is fully anticipated that the halopyridine 1-I can be replaced with alternately substituted halopyridines, halopiperazines and halopyrimidines to provide the associated analogs of structure 1-V. These reactions are well-known conversions to one skilled in the art.

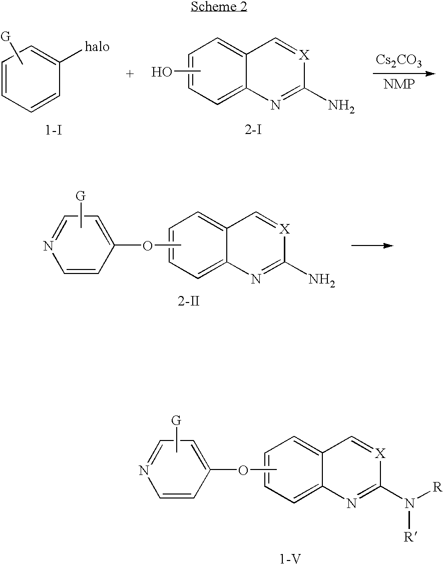

Alternative as shown in Scheme 2, compounds of the invention of formula 1-V, can be prepared by methods know to those trained in the art starting from a hydroxyl-2-aminoquinazoline (2-I, X═N), or derivative thereof, or hydroxyl-2-aminoquinoline (2-I, X═CH), or derivative thereof, and reacting with a substituted or unsubstituted a halopyridine 1-I, or derivative thereof, to form intermediates of formula 2II. Treatment of intermediate of formula 2-II, with an aldehyde or ketone in the presence of a reducing agent such as, for example, NaHB(OAc)₃ or NaH₃B(CN) in a solvent such as, for example, THF or dioxane provides compound of structure 1-V. It is fully anticipated that the halopyridine 1-I can be replaced with alternately substituted halopyridines, halopiperazines and halopyrimidines to provide the associated analogs of structure 1-V. These reactions are well-known conversions to one skilled in the art.

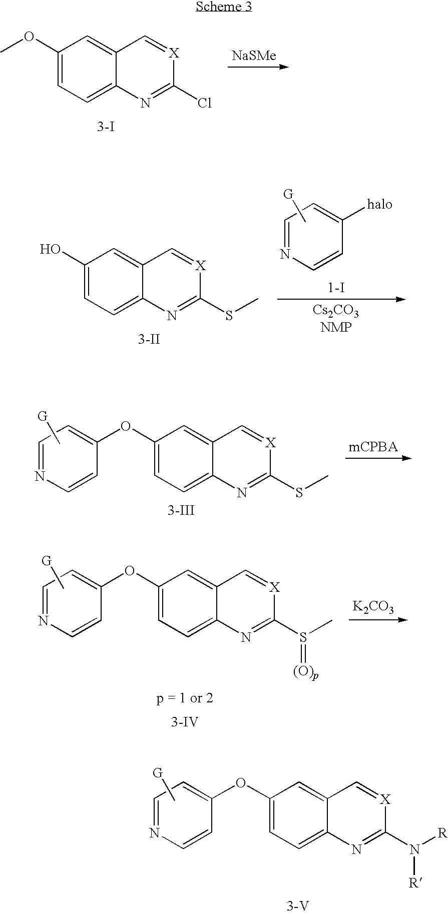

As shown in Scheme 3, compounds of the invention of formula 3-V, can be prepared by methods know to those trained in the art starting from a 6-methoxy-2-haloquinazolines (3-I, X═N), or derivative thereof, or 6-methoxy-2-haloquinolines (3I, X═CH), or derivative thereof, and reacting with a reagent such as, for example, sodium methylsulfide to form intermediates of formula 3-II. Treatment of intermediate of formula 3-II, with a halopyridine 1-I, or derivative thereof, in the presence of a base such as, for example, Cs₂CO₃ in a solvent such as, for example, NMP provides intermediates of formula 3-III. Oxidation with a reagent such as mCPBA, or equivalent, in a solvent such as CH₂Cl₂ provides intermediates of formula 3-IV. Treatment of intermediates of formula 3-IV with a primary or secondary amine in the presence of a base such as, for example, K₂CO₃ or iPr₂NEt in a solvent such as, for example, NMP or dioxane provides compounds of the invention of structure 3-V. It is fully anticipated that the halopyridine 1-I can be replaced with alternately substituted halopyridines, halopiperazines and halopyrimidines to provide the associated analogs of structure 3-V. These reactions are well-known conversions to one skilled in the art.

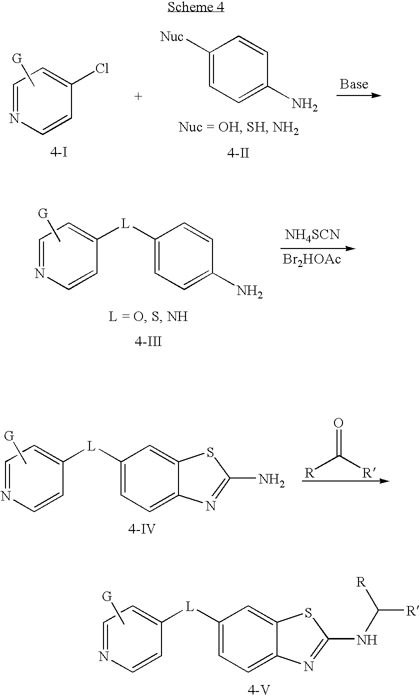

As shown in Scheme 4, compounds of the invention of formula 4-V, can be prepared by methods know to those trained in the art starting from a substituted anilines of formula 4-II, or derivative thereof, and reacting with a halopyridine, such as formula 4-I, or derivative thereof, with a base such as, for example, K₂CO₃, CsCO₃ or iPr₂NEt in a solvent such as, for example, DMF, NMP or dioxane at room temperature to 200° C. Intermediates of formula 4-III can be treated with NH₄SCN in the presence of Br₂ in HOAc at temperatures ranging from −20 to 200° C. to form intermediates of formula 4-IV. Treatment of intermediate of formula 4-IV, with an aldehyde or ketone in the presence of a reducing agent such as, for example, NaHB(OAc)₃ or NaH₃B(CN) in a solvent such as, for example, THF or dioxane provides compound of structure 4-V. It is fully anticipated that the halopyridine, such as 4-I, can be replaced with alternately substituted halopyridines, halopiperazines and halopyrimidines to provide the associated analogs of structure 4-V. These reactions are well-known conversions to one skilled in the art.

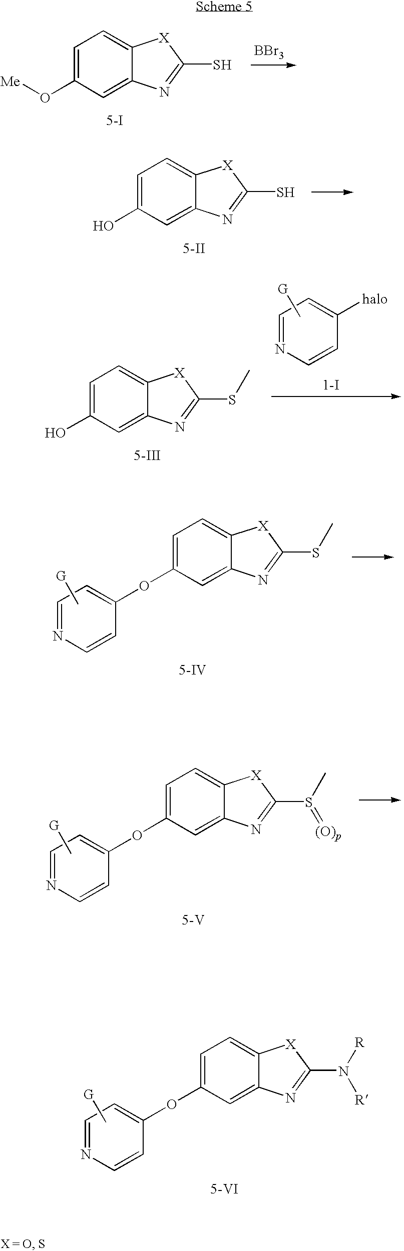

As shown in Scheme 5, compounds of the invention of formula 5-VI, can be prepared by methods know to those trained in the art starting from 6-methoxy-2-thiobenzazoles of formula 5-I. Treatment of compounds of formula 5-I with a reagent such as BBr₃, or equivalent, in a solvent such as, for example, dioxane or toluene at room temperature to 150° C. provides intermediates of formula 5-II. Treatment of intermediates of formula 5II with methyl iodide, or equivalent, in a solvent such as dichlormethane at −20° C. to room temperature provides intermediates of formula 5-III. Subsequent treatment with a halopyridine of formula 1-I, or derivative thereof, in the presence of a base such as, for example, Cs₂CO₃ in a solvent such as, for example, DMF or NMP at temperatures typically between 50 to 150° C. provides intermediates of formula 5-IV. Oxidation with a reagent such as, for example, mCPBA in a solvent such as dichloromethane at room temperature provides intermediates of formula 5-V. Treatment of intermediates of formula 5-V with a primary or secondary amine in the presence of a base such as, for example, K₂CO₃ or iPr₂NEt in a solvent such as, for example, NMP or dioxane provides compounds of the invention of structure 5-VI. It is fully anticipated that the halopyridine 1-I can be replaced with alternately substituted halopyridines, halopiperazines and halopyrimidines to provide the associated analogs of structure 5-VI. These reactions are well-known conversions to one skilled in the art.

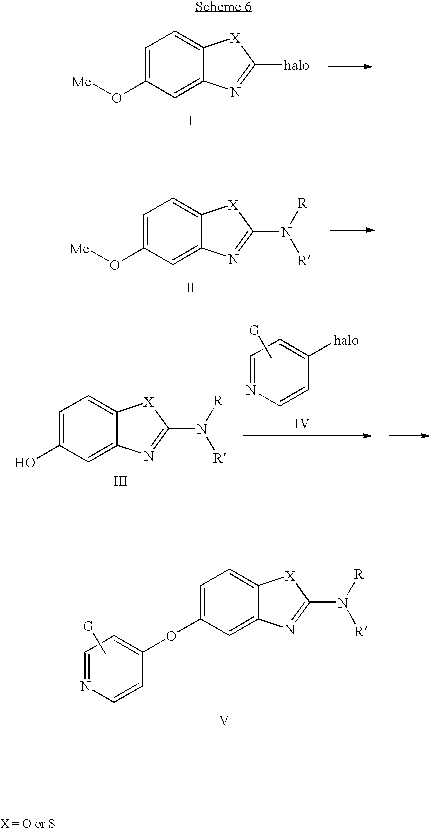

Alternatively, as shown in Scheme 6, compounds of the invention of formula 5-VI, can be prepared by methods know to those trained in the art starting from a 6-methoxy-2-halobenzazoles of formula 6-I, or derivative thereof. Reacting intermediates of formula 6-I with a primary or secondary amine in the presence of a base such as, for example, Cs₂CO₃ or iPr₂NEt in a solvent such as, for example, DMF, NMP or dioxane at temperatures typically between 90° C. to 250° C. provides intermediates of formula 6-II. Treatment of intermediates of formula 6-II with a reagent such as, for example, BBr₃ in a solvent such as, for example, dioxane or toluent at temperatures typically between room temperature to 150° C. provides intermediates of formula 6-III. Alternatively treatment of intermediates of formula II with a NaSMe, or equivalent, in a solvent such as, for example, methanol at temperatures typically between room temperature to 100° C. provides intermediates of formula 6-III. Subsequent treatment with a halopyridine of formula I-I, or derivative thereof, in the presence of a base such as, for example, Cs₂CO₃ in a solvent such as, for example, DMF or NMP at temperatures typically between 50 to 150° C. provides intermediates of formula 5-VI. It is fully anticipated that the halopyridine 1-I can be replaced with alternately substituted halopyridines, halopiperazines and halopyrimidines to provide the associated analogs of structure 5-VI. These reactions are well-known conversions to one skilled in the art.

Compounds with other five-five, five-six, six-five and six-six bicyclic systems as HET¹ in formula (I) or HET in formula (II) or the bicyclic systems in any one of formulas (III)-(VII) or in the compounds in Tables 1-4 may be prepared using general approaches and methods similar to those described above.

In addition to the generalized Schemes 1-6 provided above, compounds of the invention can be prepared following methods outlined in the Examples.

### EXAMPLES

Referring to the examples that follow, compounds of the preferred embodiments were synthesized using the methods described herein, or other methods, which are known in the art.

The compounds and/or intermediates were characterized by high performance liquid chromatography (HPLC) using a Waters Millenium chromatography system with a 2695 Separation Module (Milford, Mass.). The analytical columns were reversed phase Phenomenex Luna C18-5μ, 4.6×50 mm, from Alltech (Deerfield, Ill.). A gradient elution was used (flow 2.5 mL/min), typically starting with 5% acetonitrile/95% water and progressing to 100% acetonitrile over a period of 10 minutes. All solvents contained 0.1% trifluoroacetic acid (TFA). Compounds were detected by ultraviolet light (UV) absorption at either 220 or 254 nm. HPLC solvents were from Burdick and Jackson (Muskegan, Mich.), or Fisher Scientific (Pittsburgh, Pa.).

In some instances, purity was assessed by thin layer chromatography (TLC) using glass or plastic backed silica gel plates, such as, for example, Baker-Flex Silica Gel 1B2-F flexible sheets. TLC results were readily detected visually under ultraviolet light, or by employing well known iodine vapor and other various staining techniques.

Mass spectrometric analysis was performed on one of two LCMS instruments: a Waters System (Alliance HT HPLC and a Micromass ZQ mass spectrometer; Column: Eclipse XDB-C18, 2.1×50 mm; gradient: 5-95% (or 35-95%, or 65-95% or 95-95%) acetonitrile in water with 0.05% TFA over a 4 min period; flow rate 0.8 mL/min; molecular weight range 200-1500; cone Voltage 20 V; column temperature 40° C.) or a Hewlett Packard System (Series 1100 HPLC; Column: Eclipse XDB-C18, 2.1×50 mm; gradient: 5-95% acetonitrile in water with 0.05% TFA over a 4 min period; flow rate 0.8 mL/min; molecular weight range 150-850; cone Voltage 50 V; column temperature 30° C.). All masses were reported as those of the protonated parent ions.

GCMS analysis was or can be performed on a Hewlett Packard instrument (HP6890 Series gas chromatograph with a Mass Selective Detector 5973; injector volume: 1 μL; initial column temperature: 50° C.; final column temperature: 250° C.; ramp time: 20 minutes; gas flow rate: 1 mL/min; column: 5% phenyl methyl siloxane, Model No. HP 190915-443, dimensions: 30.0 m×25 m×0.25 m).

Nuclear magnetic resonance (NMR) analysis was performed on some of the compounds with a Varian 300 MHz NMR (Palo Alto, Calif.). The spectral reference was either TMS or the known chemical shift of the solvent. Some compound samples were run at elevated temperatures (e.g., 75° C.) to promote increased sample solubility.

The purity of some of the compounds is assessed by elemental analysis (Desert Analytics, Tucson, Ariz.).

Melting points are determined on a Laboratory Devices MeI-Temp apparatus (Holliston, Mass.).

Preparative separations are carried out using a Flash 40 chromatography system and KP-Sil, 60A (Biotage, Charlottesville, Va.), or by flash column chromatography using silica gel (230-400 mesh) packing material, or by HPLC using a Waters 2767 Sample Manager, C-18 reversed phase column, 30×50 mm, flow 75 mL/min. Typical solvents employed for the Flash 40 Biotage system and flash column chromatography are dichloromethane, methanol, ethyl acetate, hexane, acetone, aqueous ammonia (or ammonium hydroxide), and triethyl amine. Typical solvents employed for the reverse phase HPLC are varying concentrations of acetonitrile and water with 0.1% trifluoroacetic acid.

It should be understood that the organic compounds according to the preferred embodiments may exhibit the phenomenon of tautomerism. As the chemical structures within this specification can only represent one of the possible tautomeric forms, it should be understood that the preferred embodiments encompasses any tautomeric form of the drawn structure.

It is understood that the invention is not limited to the embodiments set forth herein for illustration, but embraces all such forms thereof as come within the scope of the above disclosure.

The examples below as well as throughout the application, the following abbreviations have the following meanings. If not defined, the terms have their generally accepted meanings

### Abbreviations

ACN Acetonitrile
BINAP 2,2′-bis(diphenylphosphino)-1,1′-binapthyl
DCM Dichloromethane
DIEA diisopropylethylamine
DIPEA N,N-diisopropylethylamine
DME 1,2-dimethoxyethane
DMF N,N-dimethylformamide
DMSO dimethyl sulfoxide
DPPF 1,1′-bis(diphenylphosphino)ferrocene
EtOAc ethyl acetate
EtOH ethanol
HATU 2-(7-Aza-1H-benzotriazole-1-yl)-1,1,3,3-tetramethyluronium hexafluorophosphate
HPLC high performance liquid chromatography
IPA isopropanol
mCPBA meta-chloroperoxybenzoic acid
MeOH methanol
NBS N-bromosuccinimide
NMP N-methyl-2-pyrrolidone
RT or room temp. room temperature
THF tetrahydrofuran

### Example 1

### 4-(2-((1R,2R)-2-hydroxycyclohexylamino)quinolin-6-yloxy)-n-methylpicolinamide

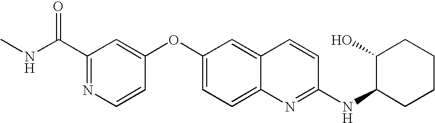

The subject compound was prepared by the general scheme below:

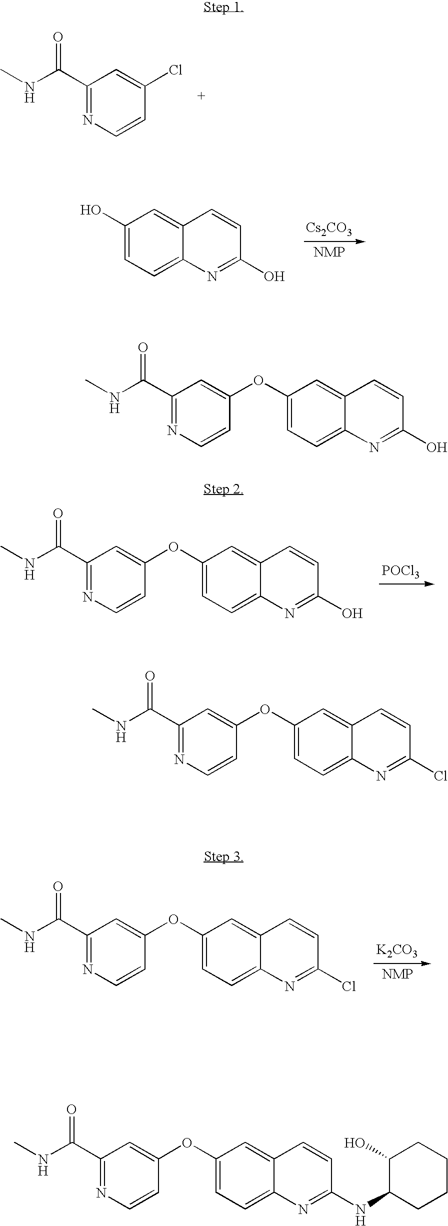

### Step 1. Preparation of 4-(2-hydroxyquinolin-6-yloxy)-N-methylpicolinamide

To a solution of 2,6-Quinolinediol (500 mg, 3.10 mmol, 1.0 eq) in 10 mL of NMP at room temp. was added Cs₂CO₃ (2.52 g, 7.75 mmol, 2.5 eq.) and 4-chloro-N-methylpicolinamide (632 mg, 3.72 mmol, 1.2 eq.) and the reaction was allowed to stir at 80° C. for ca. 12 hours. Thereafter, the reaction was quenched with water (ca. 100 mL). and extracted with EtOAc (3×100 mL). The combined organics were dried over Na₂SO₄ and condensed in vaccuo to yield the title compound which was taken to the next step without further purification. M+H=[296]

### Step 2. Preparation of 4-(2-chloroquinolin-6-yloxy)-N-methylpicolinamide

The solution of 4-(2-hydroxyquinolin-6-yloxy)-N-methylpicolinamide (600 mg, 2.03 mmol) in POCl₃ was heated in a sealed glass bomb at 80° C. for ca. 1 hour. After bringing the reaction to an ambient temperature, POCl₃ was removed in vaccuo and the residue after being neutralized with saturated NaHCO₃ solution (200 mL), was extracted with EtOAc (3×250 mL) from the aqueous phase; thereafter, the combined organic layers were dried over Na₂SO₄ and condensed under vacuum to yield the title compound which was pure enough for the next reaction. M+H=[314.0]

### Step 3. Preparation of 4-(2-((1R,2R)-2-hydroxycyclohexylamino)quinolin-6-yloxy)-Nmethyl picolinamide

To a solution of 4-(2-chloroquinolin-6-yloxy)-N-methylpicolinamide (25 mg, 0.079 mmol) in ca. 1 mL NMP was added Hunig's Base (41 μL, 0.237 mmol, 3 eq) and (1R,2R)-2-amino cyclohexanol-HCl (18 mg, 0.119 mmol., 1.5 eq). The reaction was allowed to stir at 110° C. for a few days. Thereafter K₂CO₃ (32 mg, 0.237 mmol, 3 eq) was added and after further heating for more than 48 hours at the same temperature, the product was isolated via reverse phase chromatography. M+H=[393.1]

### Example 2

### 4-(2-((1R,2R)-2-hydroxycyclohexylamino)quinazolin-6-yloxy)-N-methylpicolinamide

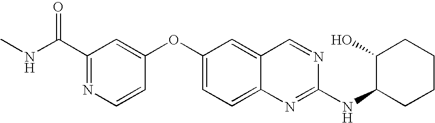

The subject compound was prepared by general scheme below:

### Step 1. Preparation of 2-(methylthio)quinazolin-6-ol

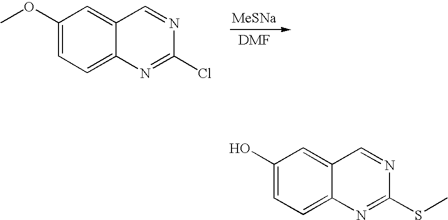

To a solution of 2-chloro-6-methoxy quinazoline (500 mg, 2.56 mmol) in DMF was added NaSMe (630 mg, 8.99 mmol, 3.5 eq) and the reaction was heated at 80° C. for ca. 16 hours. Thereafter, the reaction was quenched with water (ca. 100 mL). The aqueous layer was neutralized with few drops of HCl and was extracted with EtOAc (3×100 mL). The combined organics were dried over Na₂SO₄ and condensed in vaccuo to yield the title compound which was taken to the next step without further purification. M+H=[193.1]

### Step 2. Preparation of N-methyl-4-(2-(methylthio)quinazolin-6-yloxy)picolinamide

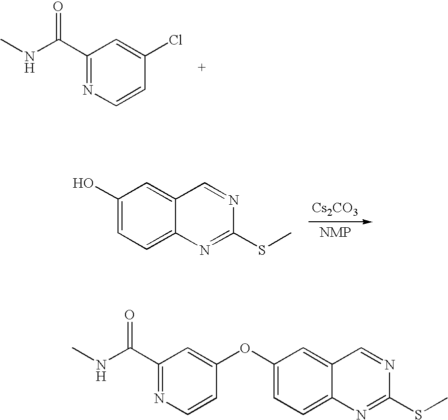

To a solution of 2-(methylthio)quinazolin-6-ol (250 mg, 1.30 mmol, 1.0 eq) in 10 mL of NMP at room temperature was added Cs₂CO₃ (1.05 g, 3.25 mmol, 2.5 eq.) and 4-chloro-N-methylpicolinamide (243 mg, 1.43 mmol, 1.1 eq.) and the reaction was allowed to stir at 80° C. for ca. 12 hours. Thereafter, the reaction was quenched with water (ca. 100 mL) and aqueous layer was extracted with EtOAc (3×100 mL). The combined organics were dried over Na₂SO₄ and condensed in vaccuo to yield the title compound which was taken to the next step without further purification. M+H=[327.1]

### Step 3. Preparation of N-methyl-4-(2-(methylsulfinyl)quinazolin-6-yloxy)picolinamide

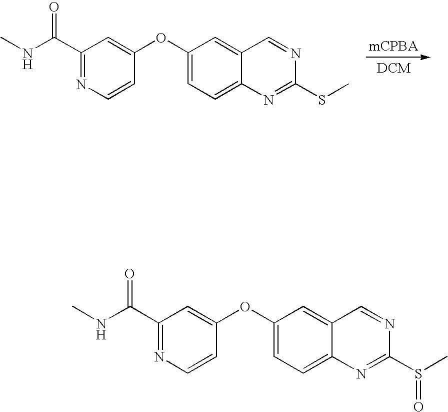

To a solution of N-methyl-4-(2-(methylthio)quinazolin-6-yloxy)picolinamide (423 mg, 1.30 mmol, 1.0 eq) in DCM at 0° C. was added (77%) mCPBA in small batches (319 mg, 1.42 mmol, 1.1 eq) and the reaction was allowed to stir at room temperature for 30-45 min. Thereafter, the reaction was quenched with NaHCO₃ (ca. 100 mL) and the aqueous layer was extracted with DCM (3×100 mL). The combined organics were dried over Na₂SO₄ and condensed in vaccuo to yield the title compound which was taken to the next step without further purification. M+H=[343.3]

### Step 4. Preparation of 4-(2-((1R,2R)-2-hydroxycyclohexylamino)quinazolin-6-yloxy)-N-methyl picolinamide

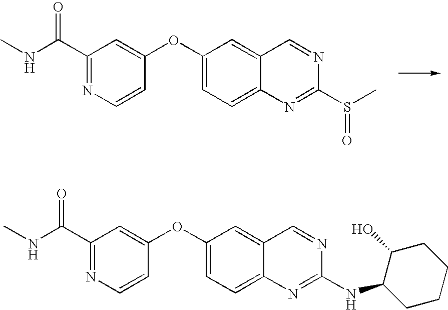

To a solution of N-methyl-4-(2-(methylsulfinyl)quinazolin-6-yloxy)picolinamide (25 mg, 0.073 mmol) in ca. 1 mL NMP was added Hunig's Base (38 μL, 0.219 mmol, 3 eq) and (1R,2R)-2-amino cyclohexanol-HCl (16 mg, 0.109 mmol, 1.5 eq) and the reaction was allowed to stir at 110° C. for ca. 16 hours and thereafter the product was isolated via reverse phase chromatography. M+H=[394.1]

### Example 3

### 4-(2-(cyclohexylmethylamino)quinazolin-6-yloxy)-N-methylpicolinamide

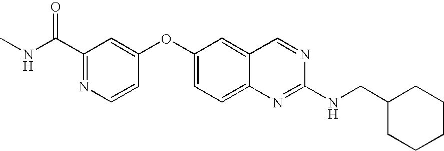

The subject compound was prepared by the general scheme below

### Step 1. Preparation of N-methyl-4-(2-(methylsulfonyl)quinazolin-6-yloxy)picolinamide

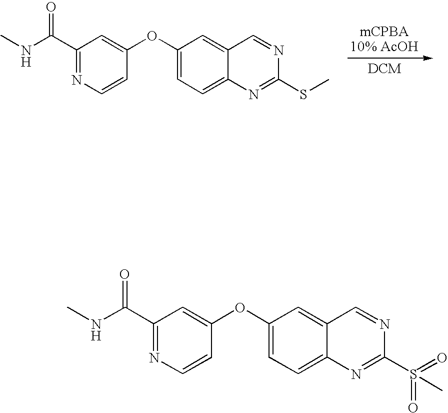

To a solution of N-methyl-4-(2-(methylthio)quinazolin-6-yloxy)picolinamide (1.0 g, 3.06 mmol, 1.0 eq) in 40.5 mL DCM at 0° C. was added 4.05 mL of AcOH (10%) and the mixture was allowed to stir for 15 min. Thereafter mCPBA (1.59 g, 9.20 mmol, 3.0 eq) was added in small batches and reaction allowed to stir at room temp. for 1 hour. The reaction was quenched with H₂O (100 mL), extracted with more DCM (3×100 mL). The combined organics were washed with a saturated solution of NaHCO₃ (1×100 mL) and brine (1×100 mL), dried over Na₂SO₄ and condensed in vaccuo to yield the title compound as a white solid, which was taken to the next step without further purification. M+H=[358]

### Step 2. Preparation of 4-(2-(cyclo hexyl methyl amino)quinazolin-6-yloxy)-N-methyl picolinamide

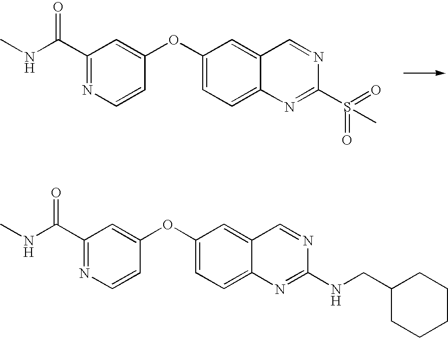

To a solution of N-methyl-4-(2-(methylsulfonyl)quinazolin-6-yloxy)picolinamide (100 mg, 0.279 mmol) in ca. 1 mL DMF was added cyclohexylmethanamine (363 μL, 2.79 mmol., 10 eq) and the reaction was heated in microwave at 170° C. for 15 min. Thereafter the product was isolated via reverse phase chromatography. M+H=[392.1]

### Example 4

### 4-(2-((2,3-dihydrobenzo[b][1,4]dioxin-5-yl)methylamino)quinazolin-6-yloxy)-N-methylpicolinamide

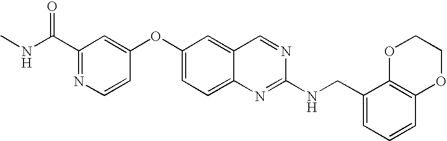

### Preparation of 4-(2-((2,3-dihydrobenzo[b][1,4]dioxin-5-yl)methylamino)quinazolin-6-yloxy)-N-methylpicolinamide

To a solution of N-methyl-4-(2-(methylsulfonyl)quinazolin-6-yloxy)picolinamide (Example 3, Step 1; 20 mg, 0.055 mmol) in ca. 400 μL DMF was added (2,3-dihydrobenzo[b][1,4]dioxin-5-yl)methanamine HCl (92 mg, 0.558 mmol, 10 eq) and TEA (78 μL, 0.558, 10 eq) and the reaction was heated in microwave at 170° C. for 15 min. Thereafter the reaction was quenched with H₂O (10 mL) and extracted with EtOAc (3×10 mL). The combined organics were washed with brine (1×10 mL) and dried over Na₂SO₄ and the product was isolated via reverse phase chromatography. M+H=[444.1]

### Example 5

### 4-(2-(2-methoxyphenylamino)quinazolin-6-yloxy)-N-methylpicolinamide

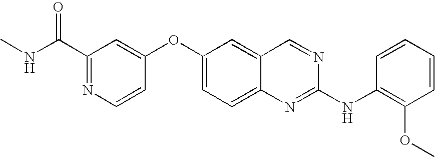

### Preparation of 4-(2-(2-methoxy phenylamino)quinazolin-6-yloxy)-N-methyl picolinamide

To a solution of N-methyl-4-(2-(methylsulfonyl)quinazolin-6-yloxy)picolinamide (Example 3, Step 1; 10 mg, 0.027 mmol) in ca. 1 mL DMF was added NaH (1.2 mg, 0.054 mmol, 2 eq) and 2-methoxyaniline (17 μL, 0.139 mmol, 5 eq) and the reaction was heated in microwave at 170° C. for 10 min. Thereafter the reaction was quenched with H₂O (10 mL) and extracted with EtOAc (3×10 mL). The combined organics were washed with brine (1×10 mL) and dried over Na₂SO₄ and the product was isolated via reverse phase chromatography. M+H=[402.2]

### Example 6

### N-methyl-4-(2-(1-(thiazol-2-yl)ethylamino)quinazolin-6-yloxy)picolinamide

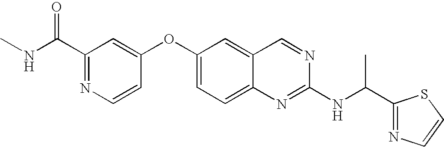

The title compound was prepared by the general scheme below

### Step 1. Preparation of 1-(thiazol-2-yl)ethanamine

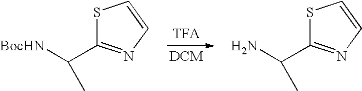

To a solution of tert-butyl 1-(thiazol-2-yl)ethylcarbamate (31.7 mg, 0.138 mmol, 1 eq) in 1 mL DCM was added 20% TFA and the reaction was stirred at room temp. for 1 hour. Thereafter, the product was dried under reduced pressure and free amine was used as is for the next step.

### Step 2. Preparation of N-methyl-4-(2-(1-(thiazol-2-yl)ethyl amino)quinazolin-6-yloxy)picolinamide

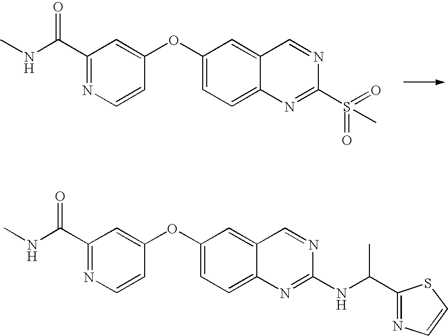

To a solution of N-methyl-4-(2-(methylsulfonyl)quinazolin-6-yloxy)picolinamide (Example 3, Step 1; 20 mg, 0.055 mmol) in ca. 1 mL DMF was added K₂CO₃ (15 mg, 0.111 mmol, 2 eq) and 1-(thiazol-2-yl)ethanamine (18 mg, 0.137 mmol, 2.5 eq) and the reaction was heated in microwave at 170° C. for 10 min. Thereafter the reaction was quenched with H₂O (10 mL) and extracted with EtOAc (3×10 mL). The combined organics were washed with brine (1×10 mL) and dried over Na₂SO₄ and the product was isolated via reverse phase chromatography. M+H=[407.1]

### Example 7

N-methyl-4-(2-(m-tolylamino)quinazolin-6-yloxy)picolinamide

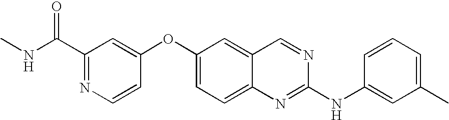

### Preparation of N-methyl-4-(2-(m-tolylamino)quinazolin-6-yloxy)picolinamide

To a solution of N-methyl-4-(2-(methylsulfonyl)quinazolin-6-yloxy)picolinamide (Example 3, Step 1; 20 mg, 0.055 mmol) in ca. 1 mL DMF was added NaH (2.6 mg, 0.110 mmol, 2 eq) and o-toluidine (12 μL, 0.110 mmol, 2 eq) and the reaction was heated in microwave at 170° C. for 10 min. Thereafter the reaction was quenched with H₂O (10 mL) and extracted with EtOAc (3×10 mL). The combined organics were washed with brine (1×10 mL) and dried over Na₂SO₄ and the product was isolated via reverse phase chromatography. M+H=[386.2]

### Example 8

N-methyl-4-(2-(2-(4-methylpiperazin-1-yl)phenylamino)quinazolin-6-yloxy)picolinamide

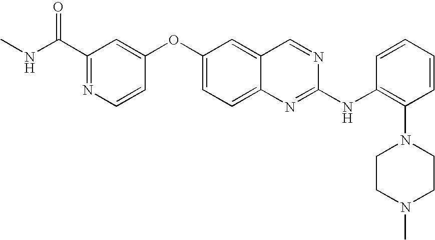

The title compound is prepared by the general scheme

### Step 1. Preparation of 1-methyl-4-(2-nitrophenyl)piperazine

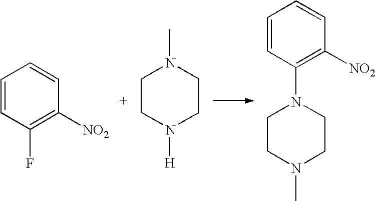

To a solution of 1-fluoro-2-nitrobenzene (100 μL, 0.949 mmol) in 1 mL NMP was added 1-methylpiperazine (126 μL, 1.13 mmol, 1.2 eq) and TEA (397 μL, 2.84 mmol, 3 eq) and the reaction was heated at 60° C. for 12 hours. Thereafter the reaction was quenched with H₂O (10 mL) and extracted with EtOAc (3×10 mL). The combined organics were washed with brine (1×10 mL) and dried over Na₂SO₄ and the crude product used as without further purification.

### Step 2. Preparation of 2-(4-methylpiperazin-1-yl)aniline

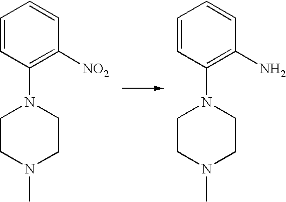

To a solution of 1-methyl-4-(2-nitrophenyl)piperazine (180 mg, 0.813 mmol) in 5 mL EtOH was added 10% Pd/C (36 mg, 20% by wt) under argon and the mixture was subjected to hydrogenation under balloon for 12 hours at room temperature. Thereafter, the reaction mixture filtered over celite and the filtrate was condensed under vacuum to yield the amine which was used as is in the next step.

### Step 3. Preparation of N-methyl-4-(2-(2-(4-methylpiperazin-1-yl)phenylamino)quinazolin-6-yloxy)picolinamide

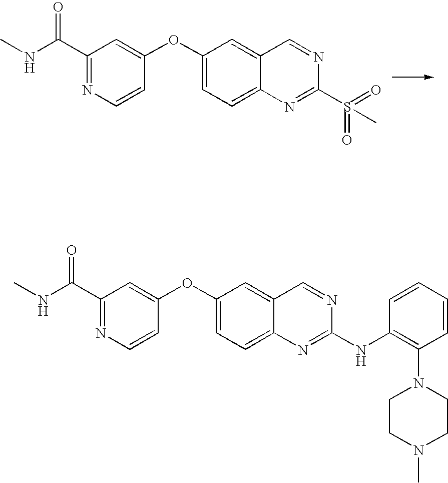

The title compound was prepared following the method described for Example 3, Step 2. M+H=[470.1]

### Example 9

### 4-(2-(cyclohexylmethoxy)quinazolin-6-yloxy)-N-methylpicolinamide

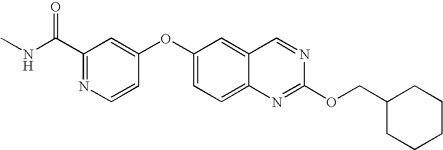

### Preparation of 4-(2-(cyclohexylmethoxy)quinazolin-6-yloxy)-N-methylpicolinamide

To a solution of N-methyl-4-(2-(methylsulfonyl)quinazolin-6-yloxy)picolinamide (Example 3, Step 1; 20 mg, 0.055 mmol) in ca. 1 mL DMF was added cyclohexylmethanol (103 μL, 0.837 mmol., 15 eq) and the reaction was heated in microwave at 170° C. for 10 min. Thereafter the reaction was quenched with H₂O (10 mL) and extracted with EtOAc (3×10 mL). The combined organics were washed with saturated NaHCO₃ solution (1×10 mL), brine (1×10 mL) and dried over Na₂SO₄ and the product was isolated via reverse phase chromatography. M+H=[393.1]

### Example 10

### 4-(2-(cyclohexanecarboxamido)quinazolin-6-yloxy)-N-methylpicolinamide

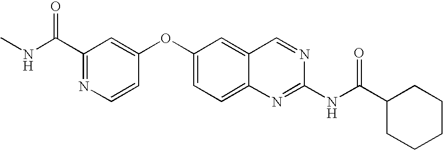

The title compound was prepared by the general scheme

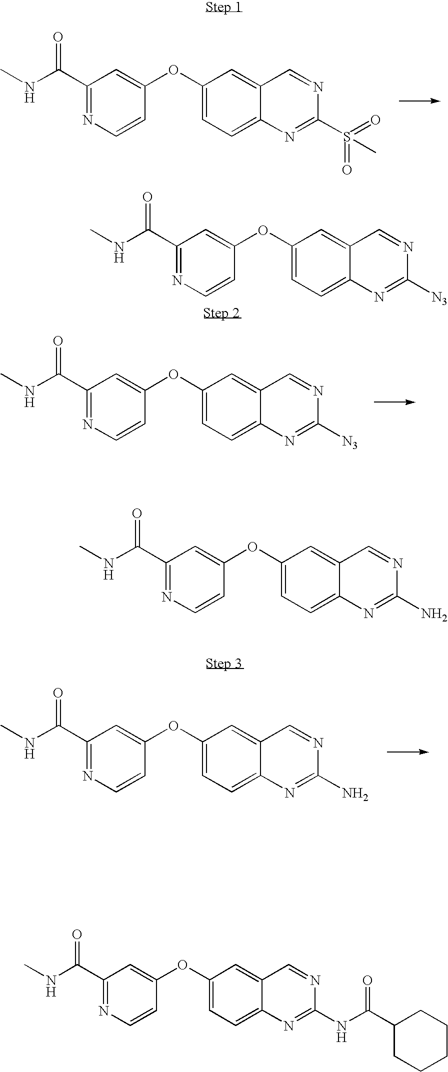

### Step 1. Preparation of 4-(2-azidoquinazolin-6-yloxy)-N-methylpicolinamide

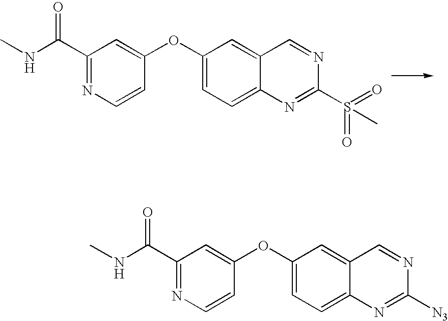

To a solution of N-methyl-4-(2-(methylsulfonyl)quinazolin-6-yloxy)picolinamide (100 mg, 0.279 mmol) in 2 mL NMP was added sodium azide (91 mg, 1.39 mmol, 5 eq) and the reaction was heated at 80° C. for 12 hours. Thereafter, H₂O (10 mL) was added and the mixture was extracted with EtOAc (3×25 mL). The combined organics were washed with saturated NaHCO₃ (1×25 mL) and brine (1×25 mL) and dried over Na₂SO₄. The product was purified via reverse phase chromatography.

### Step 2. Preparation of 4-(2-aminoquinazolin-6-yloxy)-N-methylpicolinamide

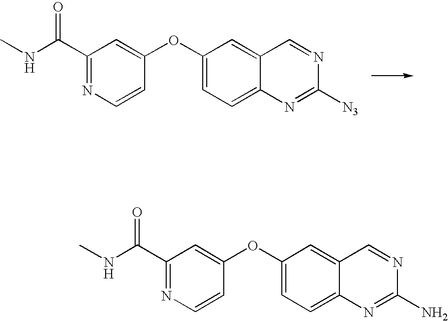

To a solution of 4-(2-azidoquinazolin-6-yloxy)-N-methylpicolinamide (200 mg, 0.622 mmol) in 1.5 mL THF and 1.5 mL H₂O was added PPh₃ (200 mg, 0.762 mmol, 1.2 eq) and the reaction was heated at 65° C. for 12 hours. Thereafter, H₂O (20 mL) was added and the pH was adjusted to 3 using 6N HCl, bringing the amine to the aqueous phase. The PPh₃O was removed by extractions with DCM (3×25 mL). The aqueous phase was treated with NH₄OH to adjust the pH to 7 and the amine was extracted into fresh DCM (3×25 mL). The combined DCM layers containing the amine were washed with brine and dried over Na₂SO₄. Removal of solvents yielded a green solid which was pure enough for the next step.

### Step 3. Preparation of 4-(2-(cyclohexanecarboxamido)quinazolin-6-yloxy)-N-methylpicolinamide

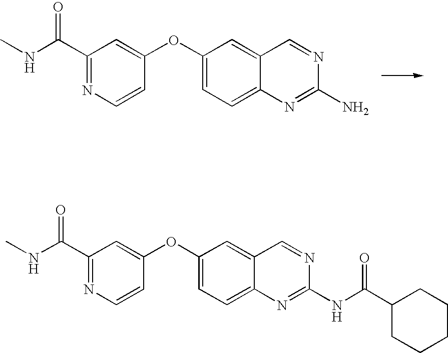

To a solution of 4-(2-aminoquinazolin-6-yloxy)-N-methylpicolinamide (18 mg, 0.060 mmol) in ca. 1 mL NMP was added cyclohexanecarbonyl chloride (50 μL, 0.365 mmol, 6 eq) and TEA (60 μL, 0.42 mmol, 7 eq). The reaction was heated in microwave at 170° C. for 10 min. Thereafter the reaction was quenched with H₂O (10 mL) and extracted with DCM (3×10 mL). The combined organics were washed with saturated NaHCO₃ solution (1×10 mL), brine (1×10 mL) and dried over Na₂SO₄ and the product was isolated via reverse phase chromatography. M+H=[406.0]

### Example 11

### 4-(2-(3-cyclohexylureido)quinazolin-6-yloxy)-N-methylpicolinamide

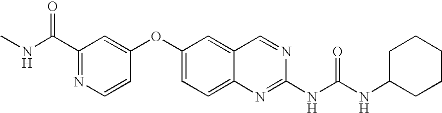

### Preparation of 4-(2-(3-cyclohexylureido)quinazolin-6-yloxy)-N-methylpicolinamide

To the solution of 4-(2-aminoquinazolin-6-yloxy)-N-methylpicolinamide (Example 10, Step 2, 18 mg, 0.060 mmol) in ca. 1 mL THF was added isocyanatocyclohexane (75 μL, 0.60 mmol, 10 eq) and the reaction was heated in microwave at 120° C. for 10 min. Thereafter the reaction was quenched with H₂O (10 mL) and extracted with DCM (3×10 mL). The combined organic layers were washed with saturated NaHCO₃ solution (1×10 mL) and brine (1×10 mL) and dried over Na₂SO₄. The product was isolated via reverse phase chromatography. M+H=[421.1]

### Example 12

### 4-(2-(2-(2-methoxyethoxy)phenylamino)quinazolin-6-yloxy)-N-methylpicolinamide

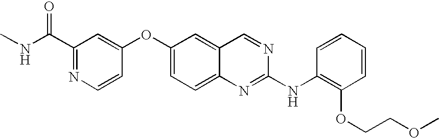

The title compound was prepared by the general scheme

### Step 1. Preparation of 1-(2-methoxyethoxy)-2-nitrobenzene

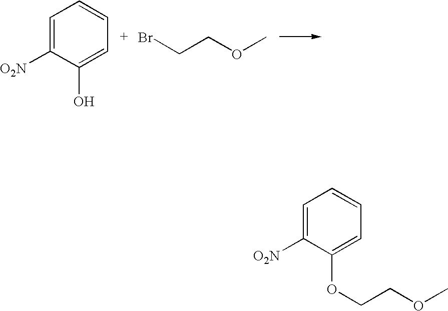

To a solution of 2-nitrophenol (1.0 g, 7.20 mmol) in 15 mL methylethyl ketone was added 1-bromo-2-methoxyethane (1.1 g, 7.9 mmol, 1.1 eq), K₂CO₃ (2 g, 14.4 mmol, 2 eq) and KI (120 mg, 0.72 mmol, 0.1 eq). The reaction was heated at 80° C. for 12 hours. Thereafter methylethyl ketone was removed under vacuum, the reaction was quenched with H₂O (50 mL) and extracted with EtOAc (3×50 mL). The combined organic layers were washed with saturated NaHCO₃ solution (1×50 mL) and brine (1×50 mL) and dried over Na₂SO₄. The crude product was used as without further purification.

### Step 2. Preparation of 2-(2-methoxyethoxy)aniline

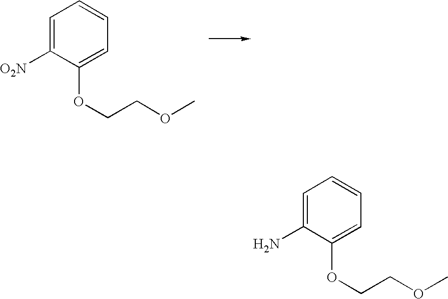

To a solution of 1-(2-methoxyethoxy)-2-nitrobenzene (766 mg, 3.88 mmol) in 10 mL of EtOH was added 10% Pd/C (153 mg, 20% by wt) under argon and the mixture was subjected to hydrogenation under balloon for 12 hours at room temp. Thereafter, the reaction mixture was filtered over celite and the filtrate was condensed under vacuum to yield the amine which was purified on Si-Gel column using DCM:MeOH:NH₃ solvent gradient in a 97:3:0.1 ratio.

### Step 3. Preparation of 4-(2-(2-(2-methoxyethoxy)phenylamino)quinazolin-6-yloxy)-N-methylpicolinamide

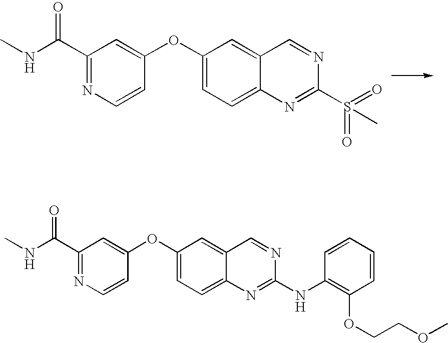

Prepared following methods described in Example 3, Step 2.

### Example 13

### 4-(2-(2-(2-hydroxyethoxy)phenylamino)quinazolin-6-yloxy)-N-methylpicolinamide

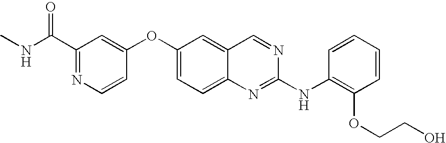

The title compound was prepared by the general scheme

### Step 1. Preparation of 2-(2-nitrophenoxy)ethanol

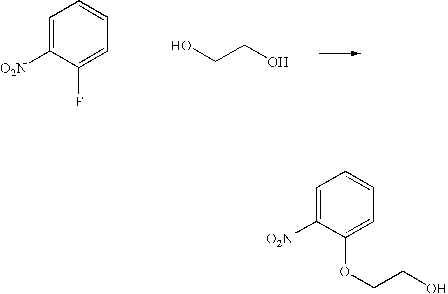

To a solution of 1-fluoro-2-nitrobenzene (2.0 g, 14.17 mmol) in 10 mL DMF was added ethylene glycol (1.0 g, 17.0 mmol, 1.2 eq) and K₂CO₃ (3.9 g, 28.4 mmol, 2 eq). The reaction was heated at 80° C. for 12 hours. Thereafter the reaction was quenched with H₂O (50 mL) and extracted with EtOAc (3×50 mL). The combined organics were washed with saturated NaHCO₃ solution (1×50 mL) and brine (1×50 mL) and dried over Na₂SO₄. The crude product was used as is without further purification.

### Step 2. Preparation of tert-butyldimethyl (2-(2-nitrophenoxy)ethoxy)silane

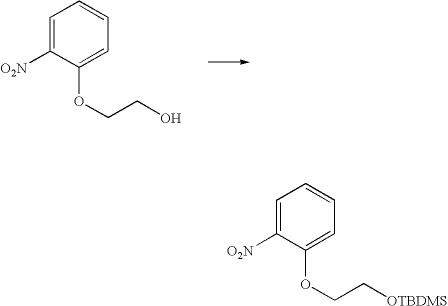

To a solution of 2-(2-nitrophenoxy)ethanol (200 mg, 1.1 mmol) in 7 mL DMF was added imidazole (150 mg, 2.2 mmol, 2.0 eq) and TBDMS-Cl (240 mg, 1.7 mmol, 1.5 eq) and DMAP (13 mg, 0.11 mmol, 0.1 eq). The reaction was stirred under N₂ for 2 hours. Thereafter the reaction was quenched with H₂O (50 mL) and extracted with EtOAc (3×50 mL). The combined organics was washed with saturated NaHCO₃ solution (1×50 mL), brine (1×50 mL) and dried over Na₂SO₄ and crude was purified on SiGel column using EtOAc:Hexane::1:9 gradient yielding pure product as a yellow solid after removal of solvents.

### Step 3. Preparation of 2-(2-(tert-butyldimethylsilyloxy)ethoxy)aniline

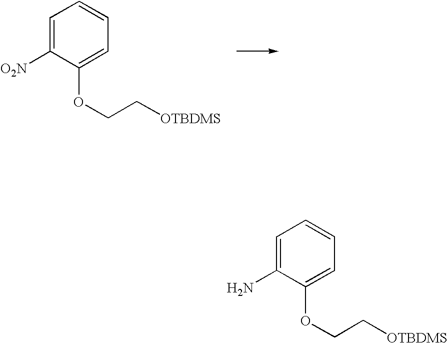

The compound was prepared following the procedure described in Step 2 of Example 12.

### Step 4. Preparation of 4-(2-(2-(2-(tert-butyl dimethyl silyloxy)ethoxy)phenylamino) quinazolin-6-yloxy)-N-methyl picolin amide

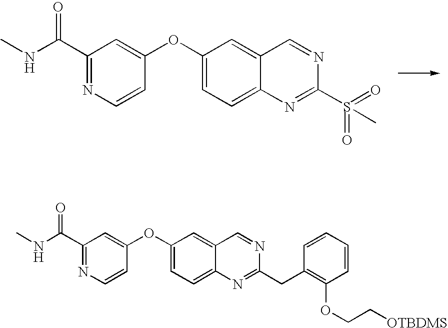

To a solution of N-methyl-4-(2-(methylsulfonyl)quinazolin-6-yloxy) picolinamide (20 mg, 0.055 mmol) in ca. 1 mL DMF was added 2-(2-(tert-butyldimethylsilyloxy)ethoxy)aniline (80 mg, 0.300 mmol, 5 eq) and the reaction was heated in microwave at 170° C. for 15 min. Thereafter the reaction quenched with H₂O (10 mL), extracted with EtOAc (3×10 mL), combined organics washed with NaHCO₃ saturated solution (1×10 mL), brine (1×10 mL) and dried over Na₂SO₄ and crude was purified on Si-Gel column using EtOAc:Hexane::1:9 gradient yielding product as brown oil.

### Step 5. Preparation of 4-(2-(2-(2-hydroxyethoxy)phenyl amino)quinazolin-6-yloxy)-N-methyl picolin amide

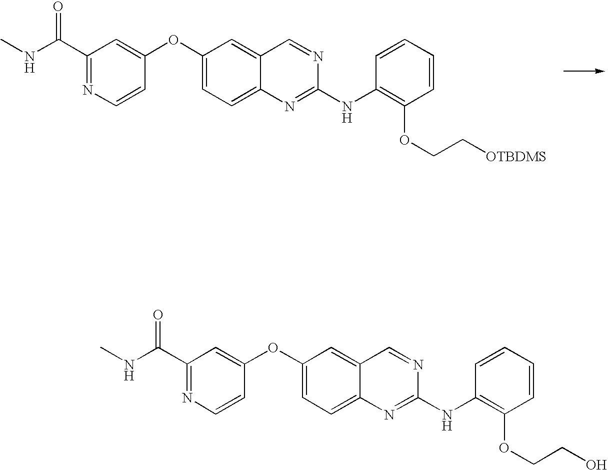

To a solution of 4-(2-(2-(2-(tert-butyl dimethyl silyloxy)ethoxy)phenylamino)quinazolin-6-yloxy)-N-methyl picolinamide (24 mg, 0.043 mmol) in ca. 1 mL THF at 0° C. was added TBAF (56 mg, 0.219 mmol, 5 eq) and the reaction was allowed to warm up to room temp. and stirred for 30 min. Thereafter the reaction was quenched with NH₄Cl (10 mL) and extracted with EtOAc (3×10 mL). The combined organics were washed with saturated NaHCO₃ solution (1×10 mL), brine (1×10 mL) and dried over Na₂SO₄. The crude material was purified on reverse phase chromatography. M+H=[432.4]

### Example 14

### 4-(2-benzamidoquinazolin-6-yloxy)-N-methylpicolinamide

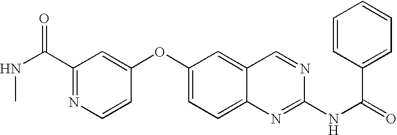

The title compound was prepared by the following steps

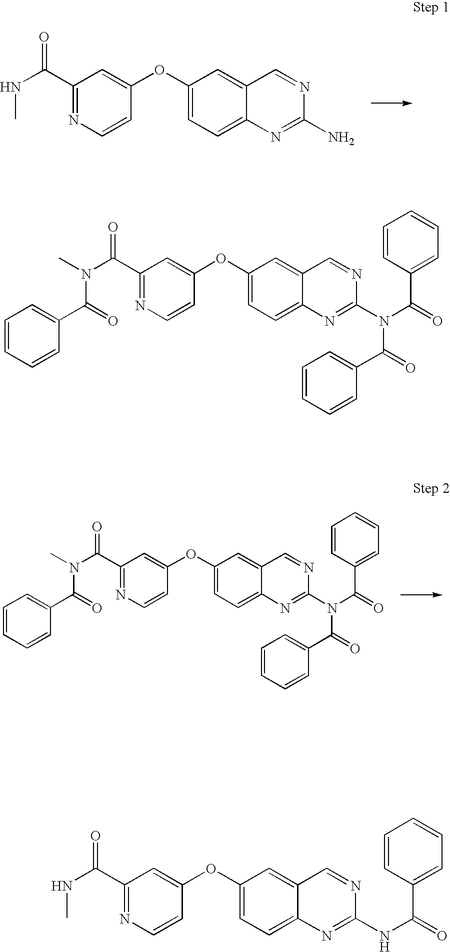

### Step 1. Preparation of N-benzoyl-4-(2-(N-benzoylbenzamido)quinazolin-6-yloxy)-N-methyl picolin amide

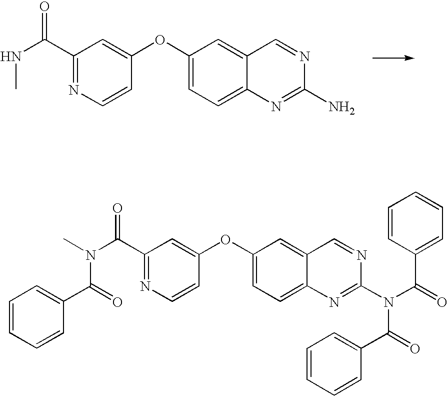

To a solution of 4-(2-aminoquinazolin-6-yloxy)-N-methylpicolinamide (Example 10, Step 2; 18 mg, 0.060 mmol) in ca. 1 mL dioxane was added benzoyl chloride (11 mg, 0.08 mmol, 1.3 eq), TEA (10 mg, 0.12 mmol, 2.0 eq) and DMAP (1 mg, 0.006 mmol, 0.1 eq). The reaction was heated at 100° C. for more than 6 hours. Thereafter the reaction was quenched with H₂O (10 mL) and extracted with EtOAc (3×10 mL). The combined organics were washed with saturated NaHCO₃ solution (1×10 mL), brine (1×10 mL) and dried over Na₂SO₄ and the product was used as is without further purification.

### Step 2. Preparation of 4-(2-benzamidoquinazolin-6-yloxy)-N-methylpicolinamide

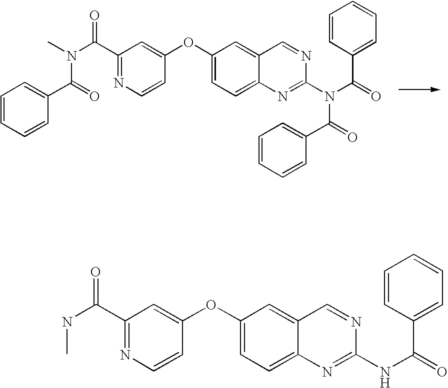

To a solution of N-benzoyl-4-(2-(N-benzoylbenzamido)quinazolin-6-yloxy)-N-methyl picolinamide (30 mg, 0.049 mmol) in ca. 1 mL THF was added 500 μL of 1N NaOH and the reaction was stirred at room temp for 45 min. Thereafter the reaction was quenched with H₂O (5 mL) and extracted with EtOAc (3×10 mL). The combined organics were washed with saturated NaHCO₃ solution (1×10 mL), brine (1×10 mL) and dried over Na₂SO₄ and the product was purified with reverse phase chromatography. M+H=[400.1]

### Example 15

### 2-N-Substituted 4-(2-aminoquinazolin-6-yloxy)-N-methyl picolinamides

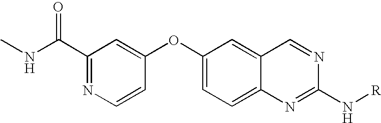

The title compound was prepared by the general procedure in the final step as follows

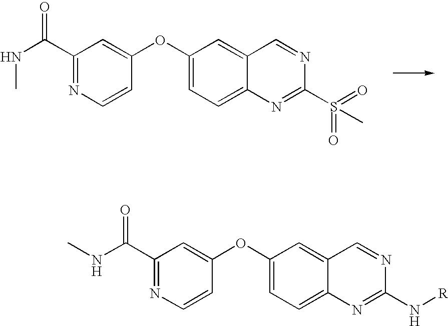

General Method: To a solution of N-methyl-4-(2-(methylsulfonyl)quinazolin-6-yloxy)picolinamide (1 eq) in ca. 1 mL DMF was added amine NH₂R (2 eq to 5 eq) and the reaction was heated in microwave at 170° C. for 15 min. Thereafter the product was isolated via reverse phase chromatography.

### Example 16

### Synthesis of 4-(2-(2-morpholinophenylamino)quinazolin-6-yloxy)-N,N-dimethylpyridine-2-carboxamide

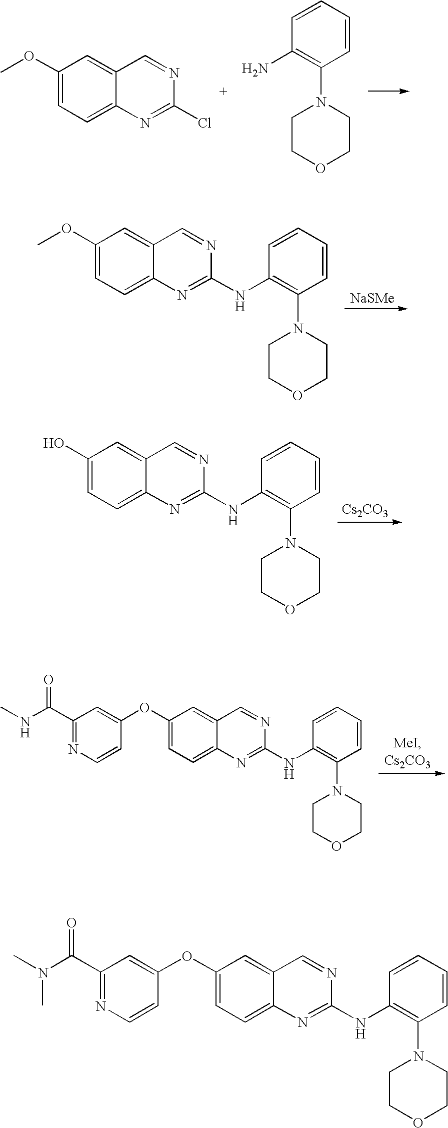

### Step 1. Synthesis of 6-methoxy-N-(2-morpholinophenyl)quinazolin-2-amine

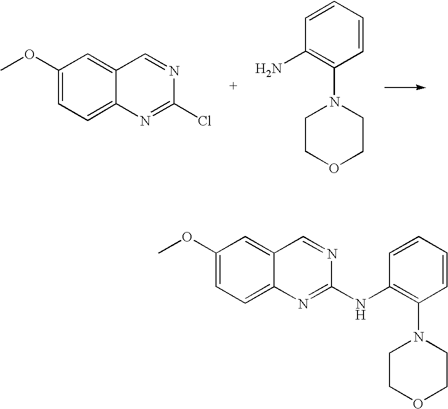

To a solution of the 2-chloro-6-methoxyquinazoline (4.0 g, 20.6 mmol) in 80 mL of ethanol was added the 2-morpholinoaniline (7.33 g, 41.1 mmol). The reaction mixture was stirred under reflux for two days. The solvent was removed. To the residue was added 100 mL of ethyl acetate and 20 mL of aq. sodium bicarbonate. The resulting mixture was stirred for 5 minutes. The organic layer was separated and washed with water and brine, dried over MgSO₄, filtered, and concentrated. The crude product was purified by column chromatography to give the titled compound. MS: MH⁺=337.2.

### Step 2: Synthesis of 2-(2-morpholinophenylamino)quinazolin-6-ol

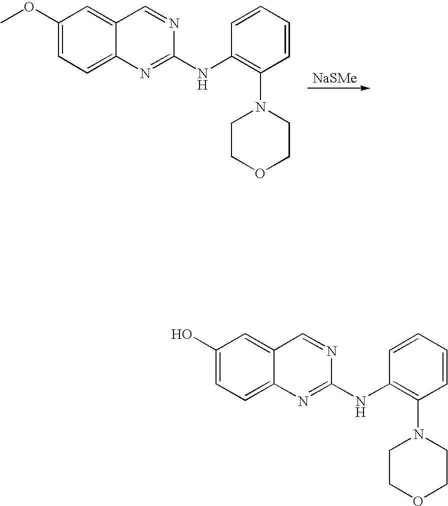

To a solution of the 6-methoxy-N-(2-morpholinophenyl)quinazolin-2-amine (648 mg, 1.93 mmol, 1.0 eq) in 10 mL of N,N-dimethylformamide was added sodium thiomethoxide (1.35 g, 19.3 mmol, 10 eq) at 155° C. The mixture was stirred at that temperature for 50 minutes. After the mixture was cooled to room temperature, 20 mL of saturated aqueous ammonium chloride solution was added. The resulting mixture was extracted with ethyl acetate (2×60 mL). The combined organic layers were washed with water (10 mL), brine (10 mL), then dried over MgSO₄, filtered, and evaporated under reduced pressure to give crude product, which was purified by silica gel column eluted with ethyl acetate and hexane to give the titled compound. MS: MH⁺=323

### Step 3: Synthesis of 4-(2-(2-morpholinophenylamino)quinazolin-6-yloxy)-N-methylpyridine-2-carboxamide

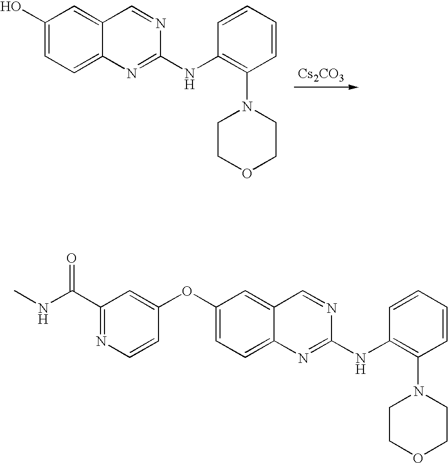

A solution of 2-(2-morpholinophenylamino)quinazolin-6-ol (320 mg, 1.0 mmol, 1 eq), 4-chloro-N-methylpyridine-2-carboxamide (179 mg, 1.05 mml, 1.05 eq) and cesium carbonate (587 mg, 1.8 mmol, 1.8 eq) in 2 mL of N,N-dimethylformaide was heated in the microwave at 150° C. for 1200 seconds. To the mixture was added 5 mL of water and the resulting mixture was extracted with ethyl acetate (3×50 mL). The combined organic layers were washed with water (10 mL), brine (10 mL), then dried over MgSO₄, filtered, and evaporated under reduced pressure to give crude product, which was purified by silica gel column eluted with ethyl acetate and hexane to give the titled compound. MS: MH⁺=457

### Step 4: Synthesis of 4-(2-(2-morpholinophenylamino)quinazolin-6-yloxy)-N,N-dimethylpyridine-2-carboxamide

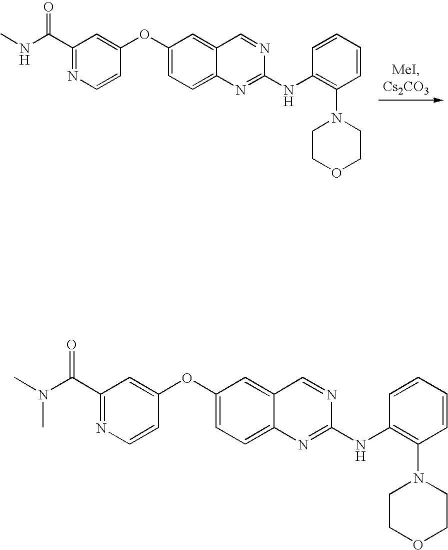

To a solution of the 4-(2-(2-morpholinophenylamino)quinazolin-6-yloxy)-N-methylpyridine-2-carboxamide (15 mg, 0.0328 mmol, 1.0 eq) in 2 mL of N,N-dimethylformamide was added cesium carbonate (21.4 mg, 0.0656 mmol, 2.0 eq) and methyl iodide (9.3 mg, 0.0656 mmol, 2.0 eq) at room temperature. The reaction mixture was stirred at that temperature overnight. To the mixture was added 5 mL of water. The resulting mixture was extracted with ethyl acetate (3×20 mL). The combined organic layers were washed with brine (10 mL), then dried over MgSO₄, filtered, and evaporated under reduced pressure to give the crude product, which was purified by preparative TLC sheet to give the titled compound. MS: MH⁺=471.

### Example 17

### N-(2-Morpholinophenyl)-6-(pyridin-4-yloxy)quinazolin-2-amine

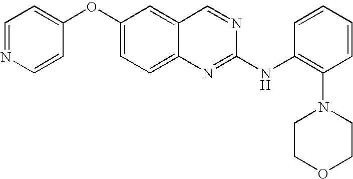

The title compound was prepared following methods described in Example 16, Step 3. MS: MH⁺=400.

### Example 18

### 6-(2-Aminopyridin-4-yloxy)-N-(2-morpholinophenyl)quinazolin-2-amine

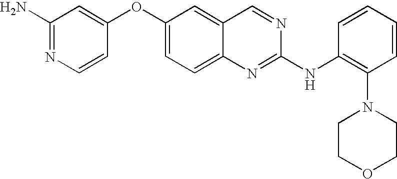

The title compound was prepared following methods described in Example 16, Step 3. MS: MH⁺=415.

### Example 19

### Synthesis of N-(4-(2-(2-morpholinophenylamino)quinazolin-6-yloxy)pyridin-2-yl)acetamide

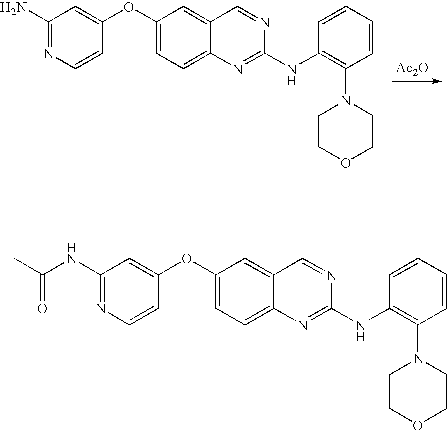

To a solution of 6-(2-aminopyridin-4-yloxy)-N-(2-morpholinophenyl)quinazolin-2-amine (Example 18, 5.0 mg, 0.013 mmol, 1.0 eq) in 2 mL of methylene chloride was added acetic anhydride (2.6 mg, 0.026 mmol, 2.0 eq) and triethyl amine (5.2 mg, 0.051 mmol, 4.0 eq) at room temperature. The reaction mixture was stirred at that temperature overnight. Then the mixture was diluted with 20 mL of methylene chloride. The resulting mixture was washed with aqueous sodium bicarbonate (5 mL), brine (5 mL), then dried over MgSO₄, filtered, and evaporated under reduced pressure to give the crude product, which was purified by preparative TLC sheet to give the titled compound. MS: MH⁺=457.

### Example 20

### Synthesis of 4-(2-(2-morpholinophenylamino)quinazolin-6-yloxy)pyridine-2-carboxamide

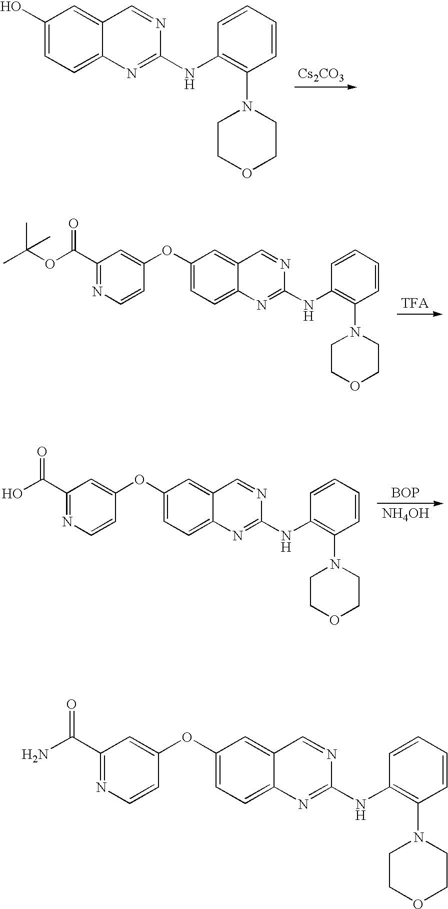

### Step 1. Synthesis of tert-butyl 4-(2-(2-morpholinophenylamino)quinazolin-6-yloxy)pyridine-2-carboxylate

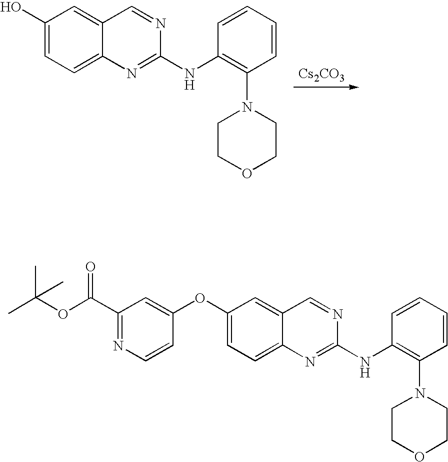

A solution of 2-(2-morpholinophenylamino)quinazolin-6-ol (32 mg, 0.10 mmol, 1.0 eq), tert-butyl 4-chloropyridine-2-carboxylate (42 mg, 0.20 mml, 2.0 eq) and cesium carbonate (97 mg, 0.30 mmol, 3.0 eq) in 1 mL of N,N-dimethylacetamide was heated in the microwave at 160° C. for 1500 seconds. To the mixture was added 5 mL of water and the resulting mixture was extracted with ethyl acetate (3×50 mL). The combined organic layers were washed with water (10 mL), brine (10 mL), then dried over MgSO₄, filtered, and evaporated under reduced pressure to give the crude product, which was purified by silica gel column eluted with ethyl acetate and hexane to give the titled compound. MS: MH⁺=500.

### Step 2. Synthesis of 4-(2-(2-morpholinophenylamino)quinazolin-6-yloxy)pyridine-2-carboxylic acid

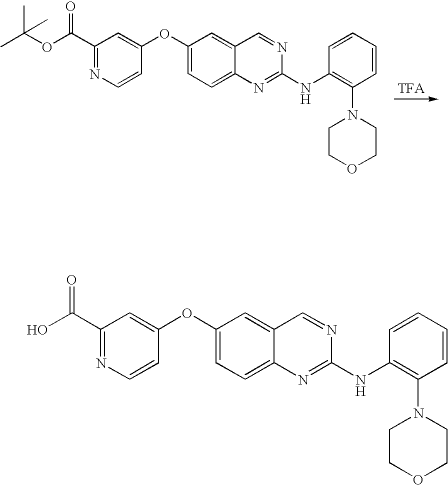

To a solution of tent-butyl 4-(2-(2-morpholinophenylamino)quinazolin-6-yloxy)pyridine-2-carboxylate in 3 mL of methylene chloride was added 1 mL 2,2,2-trifluoroacetic acid. The reaction mixture was stirred at room temperature overnight. Then additional 1 mL of 2,2,2-trifluoroacetic acid was added. After the reaction mixture was stirred for 3 hours, the solvents were removed. The crude product was used to the next step directly without further purification. MS: MH⁺=444.

### Step 3. Synthesis of 4-(2-(2-morpholinophenylamino)quinazolin-6-yloxy)pyridine-2-carboxamide

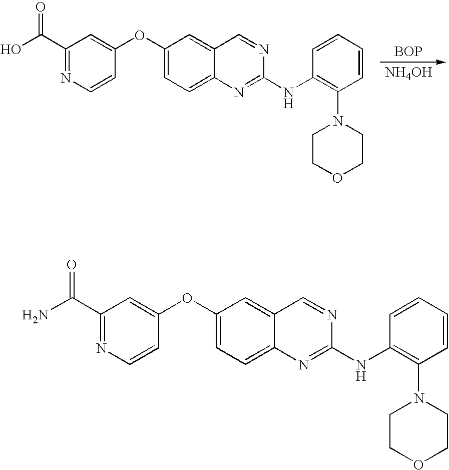

To a solution of the 4-(2-(2-morpholinophenylamino)quinazolin-6-yloxy)pyridine-2-carboxylic acid (11 mg, 0.025 mmol, 1.0 eq) in 1 mL of N,N-dimethylformamide was added benzotriazol-1-yloxytris(dimethylamino)-phosphonium hexafluorophosphate (22 mg, 0.050 mmol, 2.0 eq) and ammonium hydroxide (28%, 0.3 mL) at room temperature. The reaction mixture was stirred at that temperature for 4 hours. The mixture was concentrated and purified by reverse phase prep HPLC to give the title compound. MS: MH⁺=443.

### Example 21

### 4-(2-(2-Morpholinophenylamino)quinazolin-6-yloxy)-N-ethylpyridine-2-carboxamide

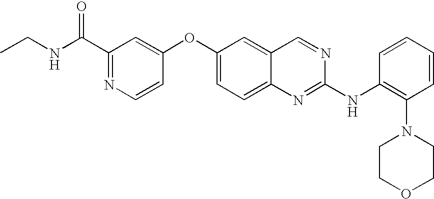

The title compound was prepared following methods described in Example 20, Step 3. MS: MH⁺=471.

### Example 22

### Synthesis of (2-(cyclohexylmethylamino)benzo[d]thiazol-6-yl)(pyridin-4-yl)methanone

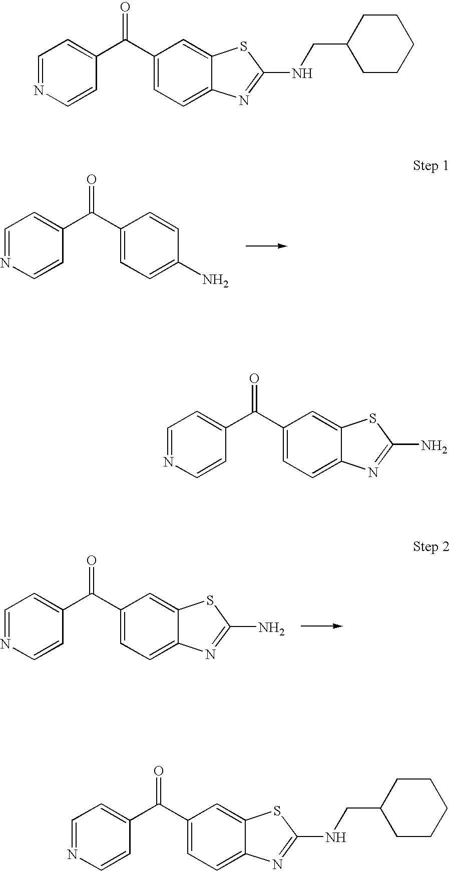

### Step 1. Preparation of (2-aminobenzo[d]thiazol-6-yl)(pyridin-4-yl)methanone

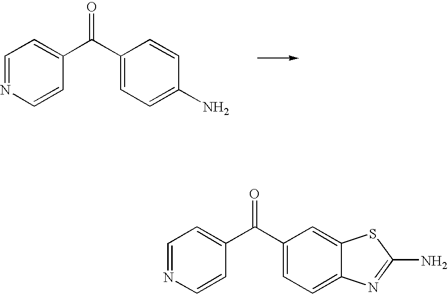

In a cold water bath, to a solution of (4-aminophenyl)(4-pyridyl)methanone (25 mg, 0.126 mmol, 1.0 eq) in 5 mL of AcOH in round bottom flask was added NH₄SCN (23 mg, 0.315 mmol, 2.5 eq) and the reaction was allowed to stir for 10 minutes after which Br₂ (24 mg, 0.151 mmol, 1.2 eq) was added and the reaction was further stirred at room temp. Reaction progress was followed via LCMS. Subsequent minor additions of NH₄SCN and Br₂ led to completion within an hour. Thereafter, AcOH was removed in vaccuo and the reaction mixture was quenched with saturated NaHCO₃ (25 mL) and the product was extracted with EtOAc (3×50 mL). The combined organic extracts were washed with water (50 mL) and brine (50 mL) and dried over Na₂SO₄. The crude product obtained was pure enough for the next step. MH⁺=256.0

### Step 2. Preparation of (2-(cyclohexylmethylamino)benzo[d]thiazol-6-yl)(pyridin-4-yl)methanone

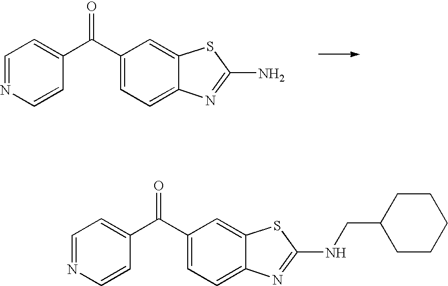

To a solution of (2-aminobenzo[d]thiazol-6-yl)(pyridin-4-yl)methanone (30 mg, 0.117 mmol, 1 eq) in 1 mL of DMF was added K₂CO₃ (32 mg, 0.234 mmol, 2 eq) and bromomethyl cyclohexane (31 mg, 0.175 mmol, 1.5 eq) and the reaction was heated at 80° C. for over 12 hours. Thereafter, the product was isolated via auto prep. MH⁺=351.47

### Example 23

### Synthesis of (2-(cyclohexylmethylamino)benzo[d]thiazol-6-yl)(pyridin-4-yl)methanol

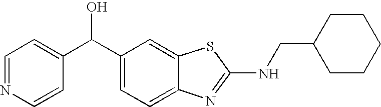

To a solution of (2-(cyclohexylmethylamino)benzo[d]thiazol-6-yl)(pyridin-4-yl)methanone (ca. 15 mg) in 1 mL EtOH was added excess NaBH₄ and the reaction was stirred at room temp. for 10-15 minutes. Thereafter, product was isolated on auto prep. MH⁺=354.1

### Example 24

### Synthesis of N-(cyclohexylmethyl)-6-(pyridin-4-ylmethyl)benzo[d]thiazol-2-amine

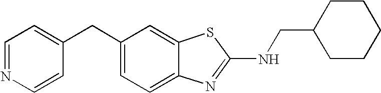

The title compound was prepared following the procedures described in Tetrahedron, 41(9):1753-1762, 1985. MH⁺=338.1

### Example 25

### 4-(2-(cyclohexylmethylamino)benzo[d]thiazol-6-ylthio)-N-methylpicolinamide

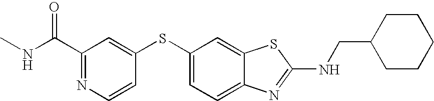

### Step 1. Preparation of 4-(4-aminophenylthio)-N-methylpicolinamide

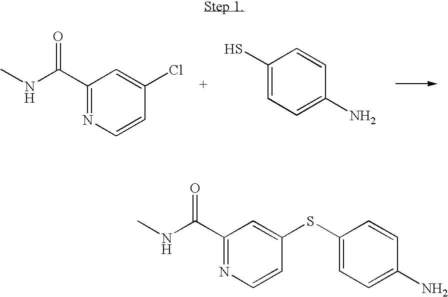

This compound was prepared following the procedures described in the Journal of Medicinal Chemistry, 48(5):1359-1366, 2005.

### Step 2 & 3

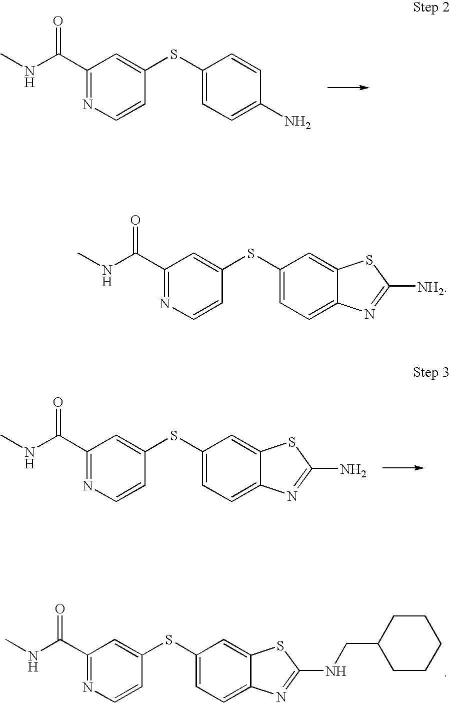

The above transformations were carried out as exemplified in Steps 1 & 2 of Example 22. MH⁺=413.1

### Example 26

### Synthesis of 4-(2-[((1R,2R)-2-hydroxycyclohexylamino)benzo[d]thiazol-6-ylamino)-N-methylpicolinamide

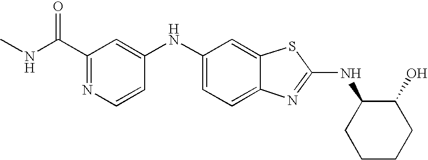

### Step 1. Preparation of N-methyl-4-(2-(methylthio)benzo[d] thiazol-6-yl amino)picolin amide

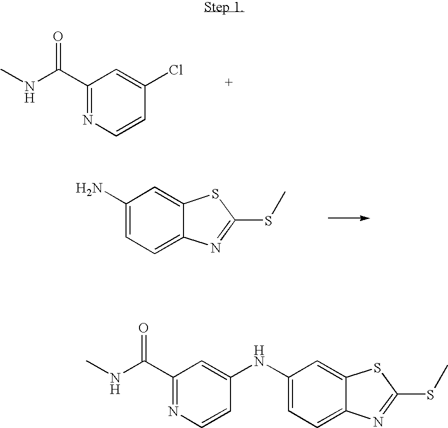

To a solution of 2-(methylthio)-1-3-benzothiazole-6-amine (423 mg, 2.48 mmol, 1.0 eq) and 4-chloro-N-methylpicolinamide (486 mg, 2.48 mmol, 1.0 eq) in 5 mL of IPA was added 500 μL of conc. HCl and 1 mL of H₂O. The reaction was allowed to stir at 90° C. for 12 hours. Thereafter, the reaction mixture was condensed in vaccuo and quenched with saturated sodium bicarbonate (50 mL), and extracted with EtOAc (3×50 mL). The combined organic extracts were washed with brine (1×50 mL) dried over Na₂SO₄ and condensed in vaccuo. Pure product was obtained after purification on ISCO using 0%-100%-EtOAc-Hexane gradient. MH⁺=331.1

### Step 2 & 3

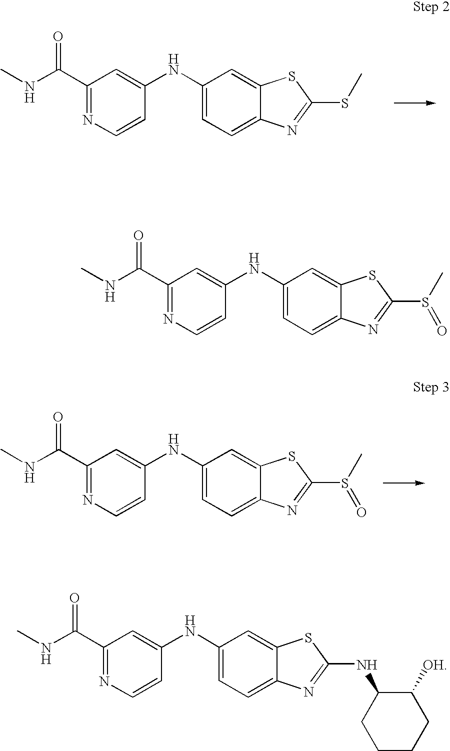

The above transformations were carried out following the methods described in Steps 2 & 3 of Example 2. MH⁺=398.1

### Example 27

### 4-(2-((1R,2R)-2-hydroxycyclohexylamino)benzo[d] thiazol-5-yloxy)-N-methylpicolinamide

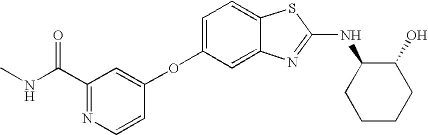

The title compound was prepared by the general scheme

### Step 1. Preparation of 2-mercaptobenzo[d]thiazol-5-ol

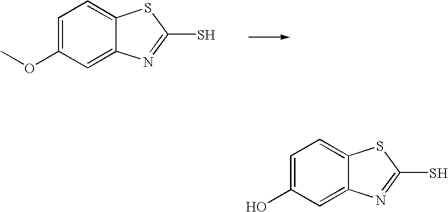

This compound was prepared following the procedures described in U.S. Pat. No. 4,873,346 -Substituted Benzothiazoles, Benzimidazoles and Benzoxazoles; Anderson, David J.; The Upjohn Company, Kalamazoo, Mich.; issued Oct. 10, 1989.

Please note that in this case the reaction was complete within 1 hour after refluxing at 110° C. in oil bath. MH⁺=184.0

### Step 2. Preparation of 2-(methylthio)benzo[d]thiazol-5-ol

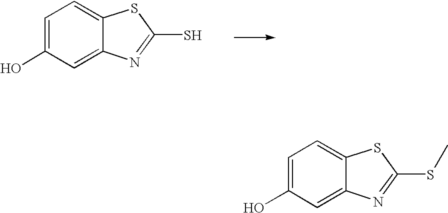

To a ice cooled solution of 2-Mercapto-benzothiazol-6-ol (250 mg, 1.37 mmol, 1.0 eq) in 5 mL of DCM at 0° C. was added triethylamine (385 μL, 2.74 mmol, 2.0 eq) followed by slow dropwise addition of iodomethane (102 μL, 1.64 mmol, 1.2 eq). After complete addition, the reaction was left stirring from 0° C. to room temp. for 30 min. The solvent was removed in vaccuo, and the reside was quenched with water (ca. 20 mL), and extraction with ethyl acetate (3×10 mL). The combined organic extracts was dried over sodium sulfate and concentrated in vaccuo to yield the desired product which was pure enough for the next step without further purification. MH⁺=198.0

### Step 3. Preparation of N-methyl-4-(2-(methylthio)benzo[d]thiazol-5-yloxy)picolinamide

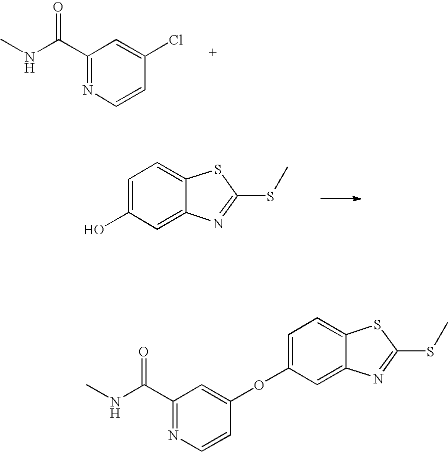

To a solution of 2-(methylthio)benzo[d]thiazol-5-ol (33 mg, 0.169 mmol, 1.0 eq) in 1 mL of NMP was added 4-chloro-N-methylpicolinamide (34 mg, 0.203 mmol, 1.2 eq) and cesium carbonate (165 mg, 0.507 mmol, 3.0 eq) at room temperature. The reaction mixture was stirred at 85° C. for 12 hours. Thereafter the mixture was diluted with water (ca. 10 mL) and the aqueous layer was extracted with ethyl acetate (ca. 30 mL×3). The combined organic layers were dried over sodium sulfate, filtered and condensed under reduced pressure to give the crude product which was purified on ISCO using a gradient of 0%-100% ethyl acetate-hexane mixture. MH⁺=[332.0]

### Step 4. Preparation of N-methyl-4-(2-(methylsulfinyl)benzo[d]thiazol-5-yloxy)picolinamide

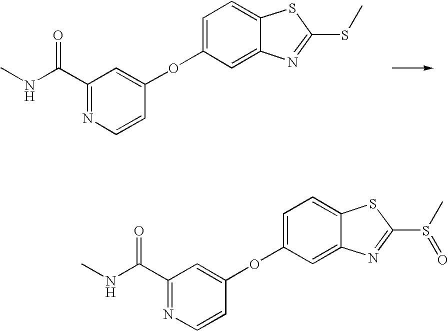

To a solution of N-methyl-4-(2-(methylthio)benzo[d]thiazol-5-yloxy)picolinamide (50 mg, 0.151 mmol, 1.0 eq) in 10 mL of DCM was added mCPBA (28 mg, 0.166 mmol, 1.1 eq) at 0° C. The reaction was stirred for 30-45 min. Thereafter, it was quenched with water (10 mL) and the aqueous phase was extracted with ethyl acetate (25 mL×5). The combined organic layers were dried over sodium sulfate, filtered and condensed under reduced pressure to yield the crude product which was sufficiently pure and was carried to the next step without further purification. MH⁺=348.0

### Step 5. Synthesis of 4-(2-[((1R,2R)-2-hydroxycyclohexylamino)benzo[d]thiazol-5-yloxy)-N-methylpicolinamide

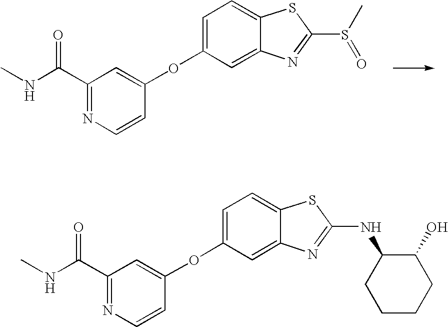

To a solution of N-methyl-4-(2-(methylsulfinyl)benzo[d]thiazol-5-yloxy)picolinamide (50 mg, 0.144 mmol, 1.0 eq) in NMP was added (1R,2R)-2-aminocyclohexanol hydrochloride (43 mg, 0.288 mmol, 2.0 eq) and DIPEA (125 μL, 0.720 mmol, 5.0 eq) and the reaction mixture was heated at 110° C. in oil bath for over 48 hours. Thereafter, the product was purified via reverse phase HPLC. MH⁺=399.0

### Example 28

### 4-(2-((1R,2R)-2-hydroxycyclohexylamino)-1H-benzo[d]imidazol-6-yloxy)-N-methylpicolinamide

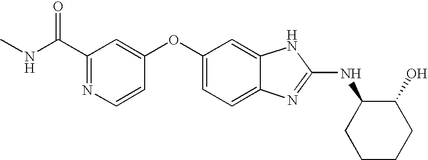

The title compound was prepared by the following steps

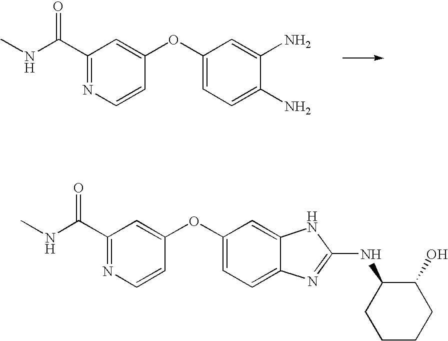

### Step 1. Preparation of 4-(2-[((1R,2R)-2-hydroxycyclohexylamino)-1H-benzo[d]imidazol-6-yloxy)-N-methylpicolinamide

To a solution of 4-(3,4-diaminophenoxy)-N-methylpicolinamide (25 mg, 0.096 mmol, 1.0 eq) in MeOH was added (1R,2R)-2-isothiocyanatocyclohexanol (15 mg, 0.096 mmol, 1.0 eq) and the reaction was stirred at room temp. for 12 hours, after which time, FeCl₃ (23 mg, 0.144 mmol, 1.5 eq) was added to the reaction mixture and the reaction was further stirred at room temperature for 3 hours. The product was isolated via reverse phase HPLC. MH⁺=382.

The compounds in the following Table 5 were prepared according to the above procedures or procedures similar to those described in the Examples above as indicated in the Method column.

TABLE 5

| No. | structure | M + H | retention | Method |
|---|---|---|---|---|
| 1 | 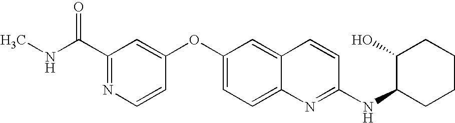 | 393.1 | 1.76 | 1 |
| 2 | 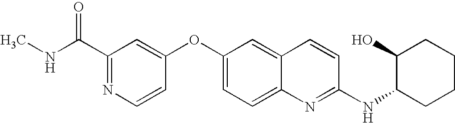 | 393.1 | 1.76 | 1 |
| 3 | 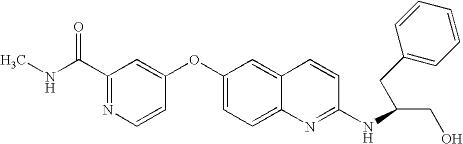 | 429.1 | 2.09 | 1 |
| 4 | 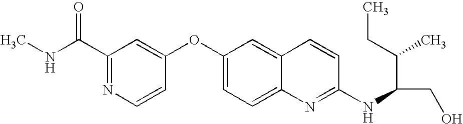 | 395.1 | 2.05 | 1 |
| 5 | 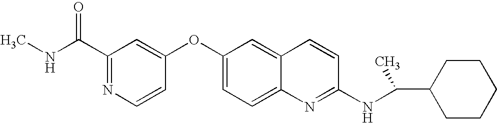 | 405.1 | 2.40 | 1 |
| 6 | 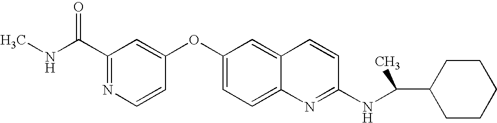 | 405.2 | 2.46 | 1 |
| 7 | 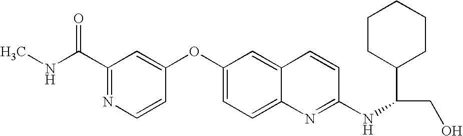 | 421.1 | 2.20 | 1 |
| 8 | 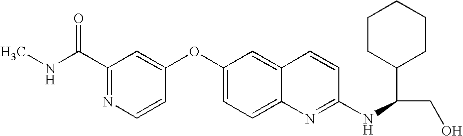 | 421.2 | 2.25 | 1 |
| 9 | 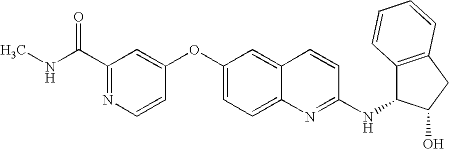 | 427.1 | 2.09 | 1 |
| 10 | 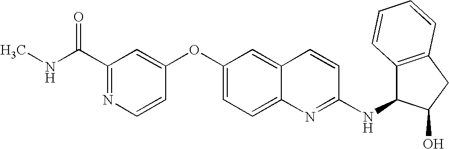 | 427.1 | 2.05 | 1 |
| 11 | 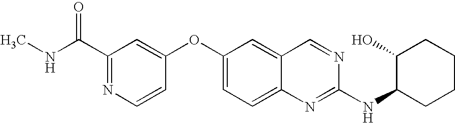 | 394.1 | 1.89 | 2 |
| 12 | 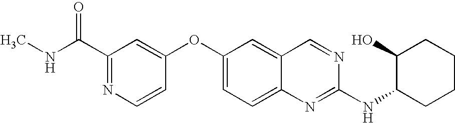 | 394.1 | 1.88 | 2 |
| 13 | 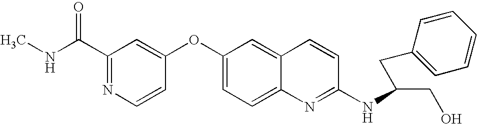 | 430.1 | 2.18 | 2 |
| 14 | 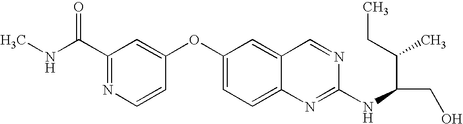 | 396.2 | 2.13 | 2 |
| 15 | 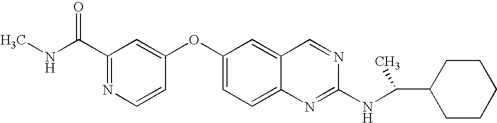 | 406.2 | 2.63 | 2 |
| 16 | 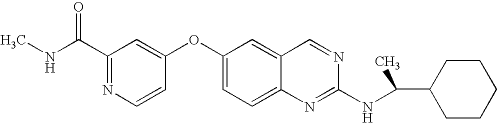 | 406.2 | 2.62 | 2 |
| 17 | 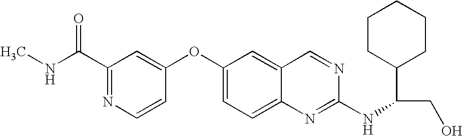 | 422.2 | 2.29 | 2 |
| 18 | 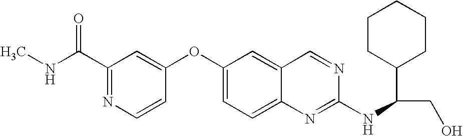 | 422.2 | 2.27 | 2 |
| 19 | 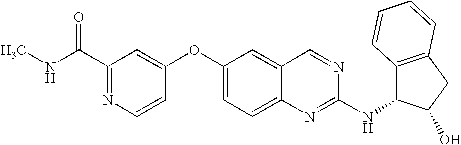 | 428.1 | 2.22 | 2 |
| 20 | 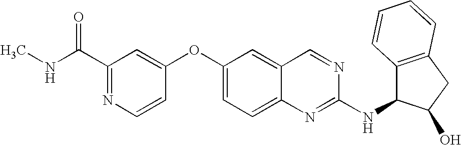 | 428.2 | 2.22 | 2 |
| 21 | 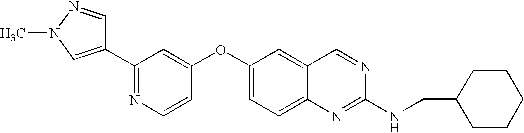 | | | 2 |
| 28 | 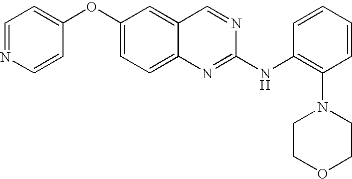 | 400 | | 17 |
| 29 | 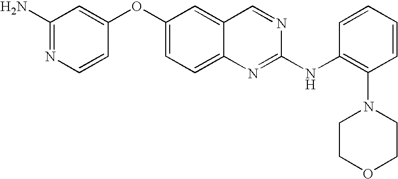 | 415 | | 18 |
| 31 | 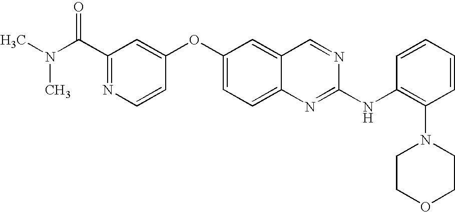 | 471 | | 16 |
| 32 | 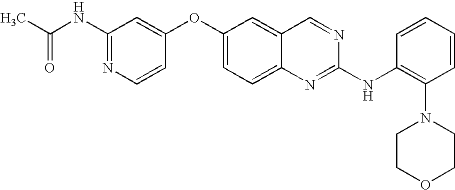 | 457 | | 19 |
| 33 | 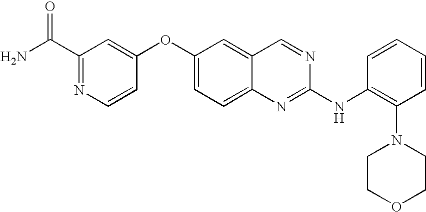 | 443 | | 20 |
| 34 | 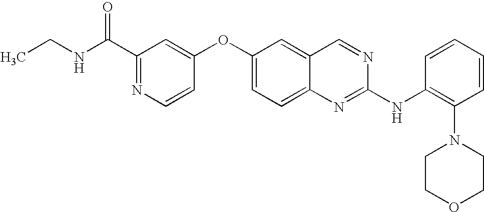 | 471 | | 20 |
| 35 | 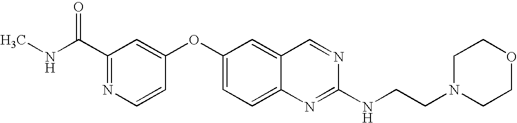 | 409 | | 3 |
| 37 | 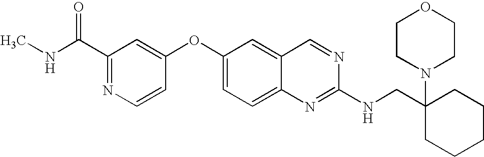 | 477.1 | | 3 |
| 38 | 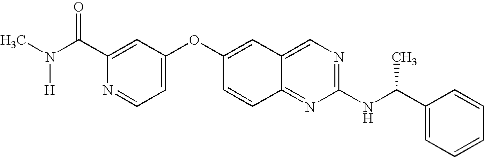 | 400.1 | | 3 |
| 39 | 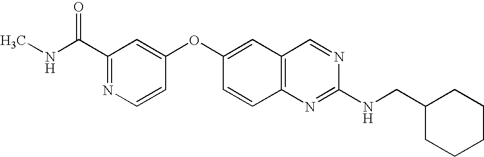 | 392.1 | | 3 |
| 40 | 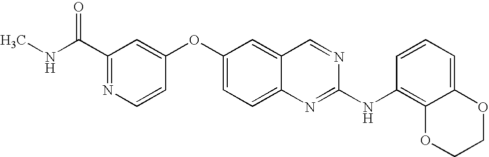 | 430.1 | | 3 |
| 41 | 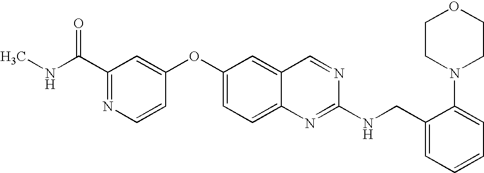 | 471.1 | | 3 |
| 42 | 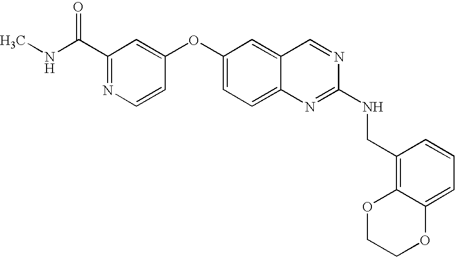 | 444.1 | | 4 |
| 43 | 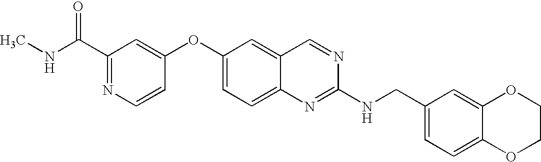 | 444.1 | | 3 |
| 44 | 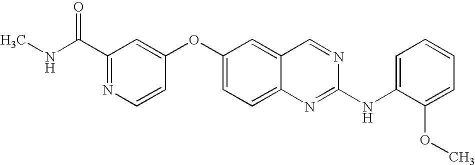 | 402.2 | | 5 |
| 45 | 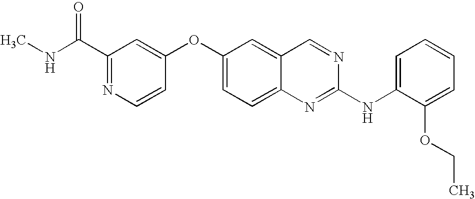 | 416.1 | | 5 |
| 46 | 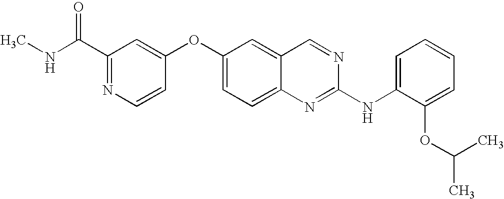 | 430.1 | | 5 |
| 48 | 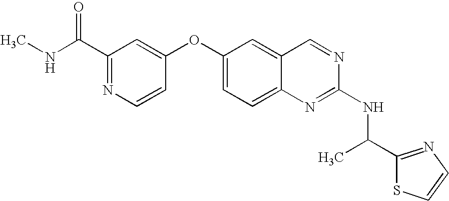 | 407.1 | | 6 |
| 52 | 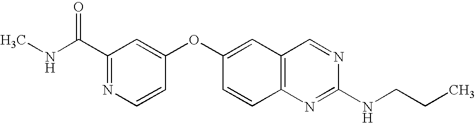 | 338.1 | | 15 |
| 53 | 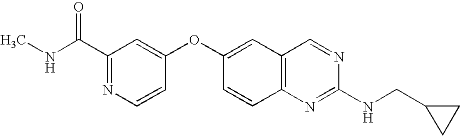 | 350.1 | | 15 |
| 54 | 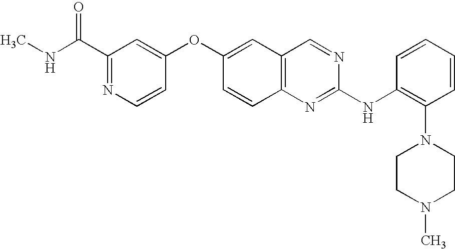 | 470.1 | | 8 |
| 55 | 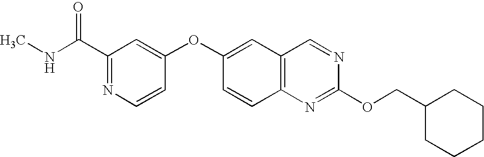 | 393.1 | | 9 |
| 56 | 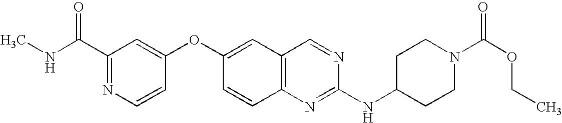 | 451.1 | | 3 |
| 57 | 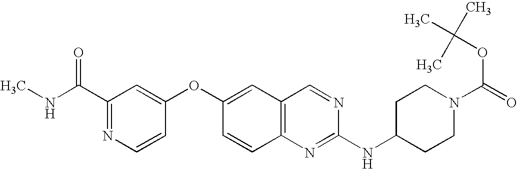 | 479.1 | | 15 |
| 58 | 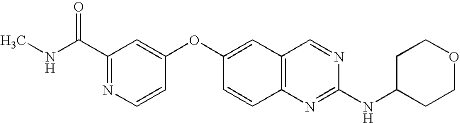 | 380.1 | | 3 |
| 59 | 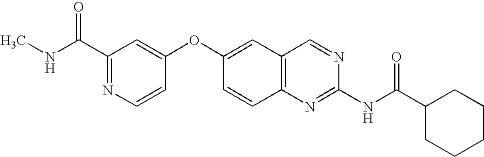 | 406 | | 10 |
| 60 | 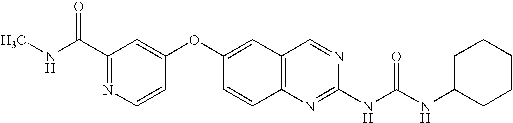 | 421.1 | | 11 |
| 61 | 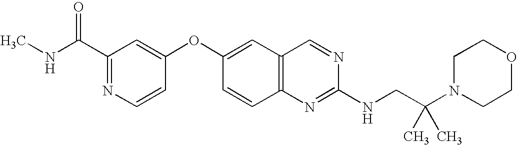 | 437.1 | | 3 |
| 63 | 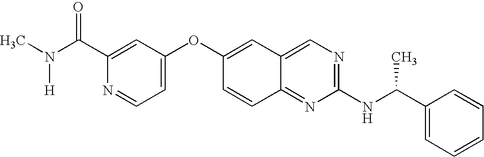 | 400.1 | | 15 |
| 64 | 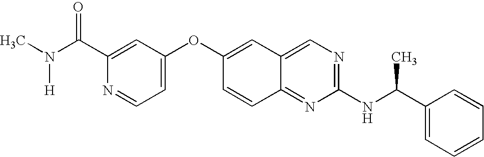 | 400.1 | | 15 |
| 66 | 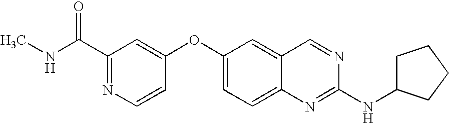 | 364.4 | | 15 |
| 67 | 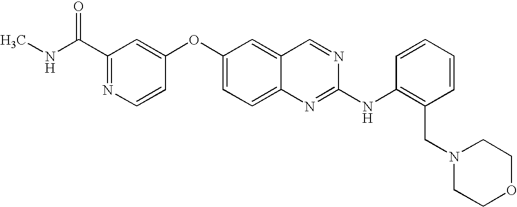 | 471.5 | | 3 |
| 68 | 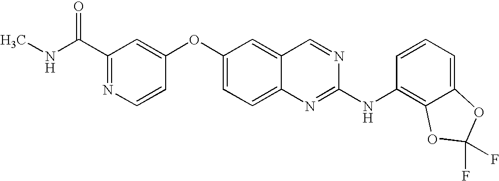 | 452.1 | | 3 |
| 69 | 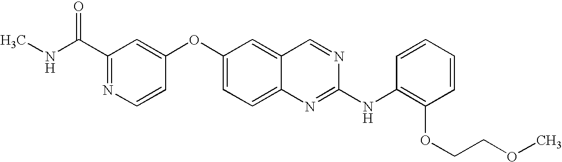 | 446.4 | | 12 |
| 70 | 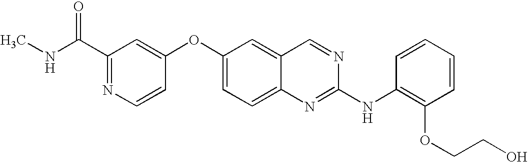 | 432.4 | | 13 |
| 71 | 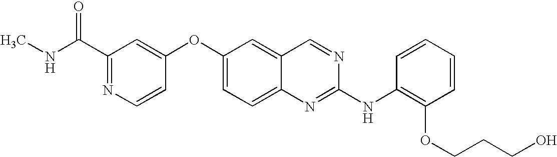 | 446.1 | | 13 |
| 72 | 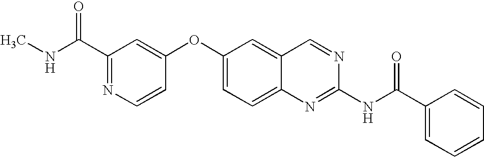 | 400.1 | | 14 |
| 73 | 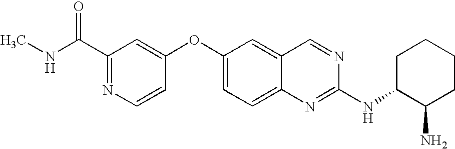 | 393.1 | | 15 |
| 74 | 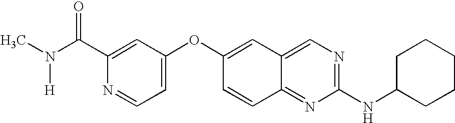 | 378.2 | | 15 |
| 76 | 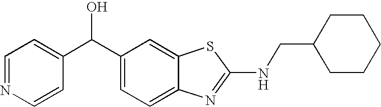 | 354 | | |
| 77 | 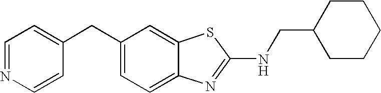 | 338.1 | | |
| 78 | 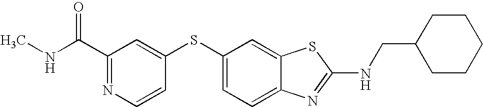 | 413.1 | | |
| 79 | 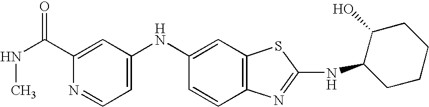 | 398.1 | | |
| 81 | 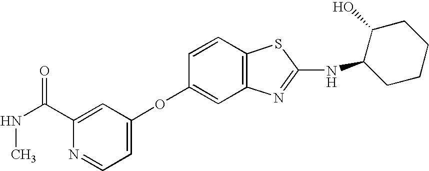 | 399.1 | | |
| 82 | 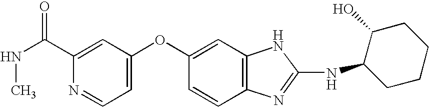 | 382.1 | | |
| 83 | 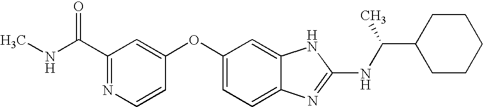 | 394.1 | | |

Each of the compounds listed in Table 5 were shown to have activity with respect to inhibition of CSF-1R with an IC₅₀ of less than about 10 μM. Many of the compounds exhibited activity with an IC₅₀ of less than about 1 μM, or less than about 0.1 μM, or less than about 0.01 μM with respect to CSF-1R inhibition. As such, each of the compounds of Tables 5 is preferred individually and as a member of a group.

In addition to CSF-1R inhibitory activity, many of the compounds of Table 5 were also screened for Raf inhibition (according to biochemical screens described in U.S. Ser. No. 10/405,945, which is entirely incorporated by reference), as well as other kinases, and shown to inhibit CSF-1R significantly greater (between about 2 and about 1,000 fold greater) than Raf and other kinases screened. More particularly, many of the compounds screened had activity greater about 1 μM with respect to Raf inhibition, whereas many of the same compounds exhibited activities with respect to CSF-1R at less than about 0.1 μM. As such, many of the compounds of Table 5 are potent and selective inhibitors of CSF-1R.

### BIOLOGICAL EXAMPLES

### Biological Example 1

### In Vitro Kinase Assays for Colony Stimulating Factor-1 Receptor (CSF-1R)

The kinase activity of various protein tyrosine kinases can be measured by providing ATP and a suitable peptide or protein tyrosine-containing substrate, and assaying the transfer of phosphate moiety to the tyrosine residue. Recombinant protein corresponding to the cytoplasmic domain of the human CSF-1R was purchased from Invitrogen Corporation, Carlsbad, Calif. U.S.A. (#PV3249). For each assay, test compounds were serially diluted, starting at 25 μM with 3 fold dilutions, in DMSO in 384 well plates then mixed with an appropriate kinase reaction buffer consisting of 50 mM Hepes, 5 mM MgCl₂, 10 mM MnCl₂, 0.1% BSA, pH 7.5, 1.0 mM dithiothreitol, 0.01% Tween 80 plus 104 ATP. Kinase protein and an appropriate biotinylated peptide substrate at 50 nM were added to give a final volume of 20 μL, reactions were incubated for 2 hours at room temperature and stopped by the addition of 10 μL of 45 mM EDTA, 50 mM Hepes pH 7.5. Added to the stopped reaction mix was 30 μL of PT66 Alphascreen beads (Perkin Elmer, Boston, Mass., U.S.A.). The reaction was incubated overnight and read on the Envision (Perkin Elmer). Phosphorylated peptide product was measured with the AlphaScreen system (Perkin Elmer) using acceptor beads coated with anti-phosphotyrosine antibody PT66 and donor beads coated with streptavidin that emit a fluorescent signal at the 520-620 nM emission wave length if in close proximity. The concentration of each compound for 50% inhibition (IC₅₀) was calculated by non-linear regression using XL Fit data analysis software.

CSF-1R kinase was assayed in 50 mM Hepes pH 7.0, 5 mM MgCl₂, 10 mM MnCl₂, 1 mM DTT, 1 mg/mL BSA, 1.0 μM ATP, and 0.05 μM biotin-GGGGRPRAATF-NH2 (SEQ ID NO:2) peptide substrate. CSF-1R kinase was added at final concentration of 4 nM.

### Biological Example 2

### In Vitro Inhibition of CSF-1R Receptor Tyrosine Phosphorylation

To test the inhibition of CSF-1R receptor tyrosine phosphorylation, HEK293H purchased from Invitrogen Cat. #11631017 cells transfected with the full-length human CSF-1R receptor cloned in house into mammalian episomal transfection vector were incubated for 1 hour with serial dilutions of compounds starting at 10 nM with 3 fold dilutions and then stimulated for 8 min with 50 ng/mL MCSF. After the supernatant was removed, the cells were lysed on ice with lysis buffer (150 mM NaCl, 20 mM Tris, pH 7.5, 1 mM EDTA, 1 mM EGTA, 1% Triton X-100 and NaF, protease and phosphatase inhibitors) and then shaken for 15-20 min at 4° C. The lysate was then transferred to total CSF-1R antibody coated 96-well plates that had already been blocked with 3% Blocker A from Mesoscale discovery (MSD) for 2 hours and washed afterwards. Lysates were incubated overnight at 4° C. and the plates were then washed 4× with MSD Tris Wash Buffer. The SULFO-TAG anti-pTyr antibody from MSD was diluted to 20 nM final in 1% Blocker A (MSD) solution and added to the washed plates and incubated for 1.5-2 h before addition of read buffer (MSD). The plates were read on the Sector 6000 instrument (MSD). Raw data was imported in Abase and EC₅₀s calculated with XL-fit data analysis software.

### Biological Example 3

### CSF-1R Inhibitors in MNFS-60 Pk/Pd Model

Five million MNFS-60 cells were implanted in HBSS/matrigel solution s.q. in the right flank. Approximately 3 weeks following tumor cell injection tumors were measured and selected mice were randomized (n=3 except for the vehicle group, where n=6) into groups based on their tumor size.

Compounds that inhibited M-CSF mediated proliferation in MNFS-60 cells and phosphorylation of CSF-1R with EC₅₀s less than 100 nM were tested in the MNFS-60 syngeneic tumor model (5×106 where implanted subcutaneously in matrigel and grown 3-4 weeks until they reached approximately 150 mm²). A single 100 mg/kg dose of representative compounds listed in Table 1 was administered to MNFS-60 tumored animals; plasma and tumor samples were harvested at various time points after dosing starting at 1 hour to up to 24 hours.

Several of the compounds disclosed herein were shown to inhibit Tyr723 phosphorylation of CSF-1R in tumor lysates at more than 50% compared to vehicle control 4 hrs after dosing as determined by Western Blot.

Additionally, several of the compounds disclosed herein were tested in a rapid onset severe arthritis mouse model (Terato, K. et al., Journal of Immunology 148:2103-2108; 1992) and treatment started on day three after injection of the anti-collagen antibody cocktail followed by LPS stimulation. Throughout the 12 days of treatment with CSF-1R inhibitors, the extent of swelling in the paws and bone resorption severity was scored. Significant attenuation of the swelling was not observed in the treated compared to control group; however, there was a trend toward improvement of bone resorption severity. There are no reports to date that CSF-1R inhibitors are effective in this arthritis model. The only successful reduction of disease progression was reported for inhibition by CSF-1R signaling with an Anti-MCSF antibody in a less severe, slower onset arthritis mouse model (Campbell et al J. Leukoc. Biol. 68: 144-150; 2000).

### Biological Example 4

### Inhibition of Raf Kinase Signaling in an In Vitro Biochemical Assay

The inhibitory effect of compounds on Raf was determined using the following biotinylated assay. The Raf kinase activity was measured by providing ATP, a recombinant kinase inactive MEK substrate and assaying the transfer of phosphate moiety to the MEK residue. Recombinant full length MEK with an inactivating K97R ATP binding site mutation (rendering kinase inactive) was expressed in E. coli and □abeled with biotin post purification. The MEK cDNA was subcloned with an N-terminal (His)6 tag and expressed in E. coli and the recombinant MEK substrate was purified from E. coli lysate by nickel affinity chromatography followed by anion exchange. The final MEK substrate preparation was biotinylated (Pierce EZ-Link Sulfo-NHS-LC-Biotin) and concentrated to 11.25 μM. Recombinant Raf (including c-Raf and mutant B-Raf isoforms) was obtained by purification from sf9 insect cells infected with the corresponding human Raf recombinant expression vectors. The recombinant Raf isoforms were purified via a Glu antibody interaction or by Metal Ion Chromatography.

For each assay, the compound was serially diluted, starting at 25 μM with 3-fold dilutions, in DMSO and then mixed with various Raf isoforms (0.50 nM each). The kinase inactive biotin-MEK substrate (50 nM) was added in reaction buffer plus ATP (1 μM). The reaction buffer contained 30 mM Tris-HCL₂ pH 7.5, 10 mM MgCl₂, 2 mM DTT, 4 mM EDTA, 25 mM beta-glycerophosphate, 5 mM MnCl₂, and 0.01% BSA/PBS. Reactions were subsequently incubated for 2 hours at room temperature and stopped by the addition of 0.5 M EDTA. Stopped reaction mixture was transferred to a neutradavin-coated plate (Pierce) and incubated for 1 hour. Phosphorylated product was measured with the DELFIA time-resolved fluorescence system (Wallac), using a rabbit anti-p-MEK (Cell Signaling) as the primary antibody and europium labeled anti-rabbit as the secondary antibody. Time resolved fluorescence can be read on a Wallac 1232 DELFIA fluorometer. The concentration of the compound for 50% inhibition (IC₅₀) was calculated by non-linear regression using XL Fit data analysis software.

### Biological Example 5

### Inhibition of cKIT and PDGFRb Kinase Signaling in an In Vitro Biochemical Assay

The IC₅₀ values for the inhibition of RTKs were determined in the alphascreen format measuring the inhibition by compound of phosphate transfer to a substrate by the respective enzyme. Briefly, the respective RTK domain purchased as human recombinant protein (cKIT Upstate #14-559, PDGFRb Invitrogen #P3082) were incubated with serial dilutions of compound in the presence of substrate and ATP concentrations within 3 times the Km of the enzyme.

The kinase domain of cKIT was assayed in 50 mM Hepes, pH=7.5, 5 mM MgCl₂, 10 mM MnCl₂, 1 mM DTT, 0.1% BSA with 0.06 uM biotinylated peptide substrate (GGLFDDPSYVNVQNL-NH2) and 15 μM ATP (ATP KM apparent =15 μM). The kinase domain of PDGFRβ was assayed in 50 mM Hepes, pH=7.5, 20 mM MgCl₂, 1 mM DTT, 0.1% BSA with 0.1 μM biotinylated peptide substrate (GGLFDDPSYVNVQNL-NH2) and 10 μM ATP (ATP KM apparent=25 μM). Reactions were incubated at room temperature for 3 to 4 hr and stopped with buffer (20 mM EDTA, 0.01% Tween-20 for both PDGFRβ and cKIT). Alphascreen PY20 beads were added to the stopped cKIT reactions and PY20 Ab/Protein A Alphascreen beads were added to the PDGFRβ stopped reactions. Both reactions were incubated overnight and read on the Alphascreen reader. The concentration of compound for 50% inhibition (IC₅₀) was calculated employing non-linear regression using XL-Fit data analysis software. As a control compound, staurosporine is run in every assay and a Z′>0.5 is required to validate results.

### Biological Example 6

### Cell Viability Assay in MCSF Dependent MNFS60 Cells

Cell viability was assessed by Cell Titer Glo, Promega. MNFS60 (murine AML cells) were seeded in TC treated 96-well plates at a density of 5,000 cells per well in RPMI-1640, 10% FBS, and 1% Penicillin Streptomycin prior to addition of compound. Test compounds were serially diluted (3 fold) in DMSO to 500× the final concentration. For each concentration of test compound, 2 μl (500×) aliquots of compound or 100% DMSO (control) were diluted in 500 μl of culture medium that contained 2× final concentration of growth factor MCSF for 2× concentration and then diluted 1× on the cells. Final concentration of MCSF is 10 ng/mL. Cells were incubated for 72 hrs at 37° C., 5% CO₂. After the incubation 100 μl Cell Titer Glo is added to each well to determine viable cells. The assay was performed according to the manufacturer's instruction (Promega Corporation, Madison, Wis. USA). Each experimental condition was performed in triplicate. Raw data was imported in Abase and EC₅₀s calculated with XL-fit data analysis software. Relative light units of wells that contained cells without MCSF in the media and as a consequence didn't grow were defined as 100% inhibited.

### Biological Example 7

### Tumor Induced Osteolysis Model

Tumor-induced osteolysis (TIO) models have been shown to recapitulate gross bone destruction seen in cancer patients with osteolytic tumor metastasis and have been reported extensively in both the bisphosphonate literature and in conjunction with the testing of novel anti-osteolytic agents. Results from these studies correlate well with human clinical activity (Kim S-J et al., 2005, Canc. Res., 65(9): 3707; Corey, E et al., 2003, Clin. Canc. Res., 9:295; Alvarez, E. et al., 2003, Clin. Canc. Res., 9: 5705). The procedure includes injection of tumor cells directly into the proximal tibia. Once the cells are established, they proliferate and secrete factors that potentiate osteoclast activity, resulting in trabecular and cortical bone resorption. Animals are treated with anti-resorptive agents following tumor cell implantation and bone destruction is measured in a number of ways at the end of the study.

The tumor cell lines utilized in this protocol are of human origin and represent tumor lines that have been previously modified such that they now express the enzyme Luciferase in order to track tumor cells in the animal using the Xenogen system. The strength of the light signal also gives an indication of approximately how many tumor cells are located at a particular site.

Mice are injected subcutaneously with either 2.5 mg/kg flunixin meglumine 30 minutes prior to cell inoculation to provide post-procedural analgesia. The mice are then be anesthetized by isoflurane inhalation (ketamine/xylazine injection may be used if isoflurane is not available). Anesthetized animals are placed in the supine position and following tumor cell aspiration into a 50 or 100 μL micro-syringe fitted with a 26- or 27-gauge needle, the needle will be inserted through the cortex of the anterior tuberosity of the right tibia with a rotating “drill-like” movement to minimize the chance for cortical fracture. Successful passage of the needle through the cortex and into the marrow is indicated by loss of resistance against the forward movement of the needle. Once the bone cortex is traversed, 10-20 μl of cell suspension (6×10̂5 MDA-MB-231Luc breast carcinoma or 3×10̂5 PC-3Mluc prostate carcinoma cells) will be injected into the tibia bone marrow. Animals will be observed to ensure uneventful recovery (warming pad or lamp) until they have recovered from anesthesia.

Progression of tumor growth in the bone can be divided into five stages (Stages 0-4). The stages are defined as follows and can be monitored by comparison to the uninjected (left) leg of the mouse:

Stage 0: normal, no sign of any change in the bone.
Stage 1: Equivocal or minimal lesion; cortex/architecture normal.
Stage 2: Definite lesion; minimal cortex/architecture disruption.
Stage 3: Large lesion; cortex/architecture disruption.
Stage 4: Gross destruction; no preservation of architecture, “late stage”. Animals reaching this stage will be taken off the study and euthanized.

Photon imaging of the legs are used to assess the tumor growth at the injection and remote sites during study using the Xenogen system to quantitate tumor cells in the tibia and confirm lack of leakage into other areas. Radiograms of the legs are taken up to once a week through the end of the study using Faxitron X-ray Unit to assess cortical bone destruction at the injection site. While using more invasive cell lines such as the PC-3M-Luc, we monitor bone damage one to two weeks after injection and weekly thereafter. For cell lines that form lesions at a slower rate, such as the MDA-MB-231 Luc, which does not manifest bone damage until 4-5 weeks post-implantation, first radiographic images are taken approximately 4 weeks after animals have been intratibially implanted with cells to establish baseline controls and then once a week to measure bone damage starting at a time point when lesions begin to develop based on model development pilot studies. For example, in mice injected with MDA-MB-231 Luc, an image would be taken approximately 4 weeks post-implantation, with weekly images thereafter.

Animals may be dosed with small molecules, monoclonal antibodies, or proteins once or twice daily, by any standard routes.

The endpoint of this study is the time point at which the majority of untreated (negative control) animals have reached late stage disease (Stage 4) and have been euthanized. At that point, the remaining animals in the study are euthanized, regardless of the stage of their tumors. Studies last approximately 5-10 weeks depending on the cell line. After the final x-ray is taken, blood is drawn from the animals by cardiac puncture (for assaying serum bone markers; see below). Endpoint x-ray images are then distributed to 5 volunteers who score each image according to the scoring system detailed above. Scores for each mouse are averaged and expressed as mean osteolytic score or percent of animals with severe osteolysis (animals with scores greater than 2).

### Biological Example 8

### Mouse Trap5b Assay (IDS Inc., Fountain Hills, Ariz.)

This assay is a solid phase immunofixed enzyme activity assay for the determination of osteoclast-derived tartrate-resistant acid phosphatase 5b in mouse serum samples. Trap5b is expressed by bone resorbing osteoclasts and secreted into the circulation. Thus, serum Trap5b is considered to be a useful marker of osteoclast activity, number and bone resorption.

The mouse Trap5b assay uses a polyclonal antibody prepared using recombinant mouse Trap5b as antigen. In the test, the antibody is incubated in anti-rabbit IgG-coated microtiter wells. After washing, standard, controls and diluted serum samples are incubated in the wells, and bound Trap5b activity is determined with a chromogenic substrate to develop color. The reaction is stopped and the absorbance of the reaction mixture read in a microtiter plate reader at 405 nm. Color intensity is directly proportional to the amount and activity of Trap5b present in the sample. By plotting the mean absorbance for each standard on the ordinate against concentration on the abscissa, values for unknown samples can be read from the standard curve and expressed in U/L Trap5b. Analytical sensitivity of the assay is 0.1 U/L and inter- and intra-assay variation are below 10%. Trap5b levels were found to correlate well with mean osteolytic score (assessed by x-ray).

While a number of the embodiments of the invention and variations thereof have been described in detail, other modifications and methods of use will be readily apparent to those of skill in the art. Accordingly, it should be understood that various applications, modifications and substitutions may be made of equivalents without departing from the spirit of the invention or the scope of the claims.

Table 6 shows the percent inhibition activities of the representative compounds of the invention when tested at about 1 μM in the indicated assays as described in the Biological Examples.

TABLE 6

| | CSF-1R % | cKit % | PDGFR % |
|---|---|---|---|
| Cmpd | Inhibition | inhibition | inhibition |
| # | at 1 μM | at 1 μM | at 1 μM |
| 1 | 100 | 51 | 33 |
| 2 | 100 | 21 | 32 |
| 3 | 98 | 64 | 41 |
| 4 | 100 | 48 | 24 |
| 5 | 100 | 91 | 62 |
| 6 | 100 | 49 | 24 |
| 7 | 98 | 31 | 16 |
| 8 | 99 | 55 | 74 |
| 9 | 100 | 61 | 3 |
| 10 | 100 | 69 | 29 |
| 11 | 99 | 23 | 21 |
| 12 | 100 | 39 | 39 |
| 13 | 88 | 28 | 24 |
| 14 | 99 | 19 | 15 |
| 15 | 100 | 50 | 26 |
| 16 | 100 | 58 | 26 |
| 17 | 100 | 61 | 38 |
| 18 | 99 | 30 | 31 |
| 19 | 100 | 30 | 3 |
| 20 | 100 | 60 | 25 |
| 21 | 100 | 100 | 91 |
| 28 | 52 | 8 | <1 |
| 29 | 90 | 9 | 1 |
| 31 | 67 | 8 | <1 |
| 32 | 99 | 7 | <1 |
| 33 | 84 | 14 | <1 |
| 34 | 80 | 14 | 2 |
| 35 | 71 | 7 | <1 |
| 37 | 99 | 16 | <1 |
| 38 | 99 | 92 | 46 |
| 39 | 99 | 10 | 52 |
| 40 | 100 | 13 | <1 |
| 41 | 100 | 6 | 92 |
| 42 | 100 | 99 | 66 |
| 43 | 98 | 24 | <1 |
| 44 | 99 | 44 | 3 |
| 45 | 99 | 38 | 12 |
| 46 | 100 | 27 | 17 |
| 48 | 98 | 18 | 15 |
| 52 | 52 | 7 | 9 |
| 53 | 83 | <1 | 9 |
| 54 | 65 | 47 | 20 |
| 55 | 99 | 42 | 6 |
| 56 | 84 | 17 | <1 |
| 57 | 93 | 12 | 2 |
| 58 | 65 | 8 | <1 |
| 59 | 99 | 13 | 13 |
| 60 | 97 | 17 | 12 |
| 61 | 93 | 12 | <1 |
| 63 | 99 | 92 | 46 |
| 64 | 99 | 16 | 7 |
| 66 | 96 | 13 | <1 |
| 67 | 96 | 13 | <1 |
| 68 | 98 | 10 | 2 |
| 69 | 99 | 17 | 5 |
| 70 | 95 | 11 | 13 |
| 71 | 90 | <1 | 11 |
| 72 | 98 | 13 | 43 |
| 73 | 62 | 8 | 19 |
| 74 | 100 | 18 | <1 |
| 76 | 65 | 49 | 27 |
| 77 | 74 | 10 | 16 |
| 78 | 100 | 13 | 34 |
| 79 | 97 | 13 | <1 |
| 81 | 99 | 39 | 71 |
| 82 | 99 | 30 | 30 |
| 83 | 100 | 100 | 100 |

The following references are cited in the specification.
Sherr, C. J., et al., The c-fins proto-oncogene product is related to the receptor for the mononuclear phagocyte growth factor, CSF 1. Cell, 1985. 41(3): p. 665-676.
Roussel, M. F., et al., Transforming potential of the c-fins proto-oncogene (CSF-1 receptor). 1987. 325(6104): p. 549-552.
Lee, P. S., et al., The Cbl protooncoprotein stimulates CSF-1 receptor multiubiquitination and endocytosis, and attenuates macrophage proliferation. Embo J, 1999. 18(13): p. 3616-28.
Inaba, T., et al., Expression of M-CSF receptor encoded by c-fins on smooth muscle cells derived from arteriosclerotic lesion. J Biol Chem, 1992. 267(8): p. 5693-9.
Baker, A. H., et al., Expression of the colony-stimulating factor 1 receptor in B lymphocytes. Oncogene, 1993. 8(2): p. 371-8.
Sawada, M., et al., Activation and proliferation of the isolated microglia by colony stimulating factor-1 and possible involvement of protein kinase C. Brain Res, 1990. 509(1): p. 119-24.
Stanley, E. R., et al., Biology and action of colony—stimulating factor-1. Mol Reprod Dev, 1997. 46(1): p. 4-10.
Bourette, R. P. and L. R. Rohrschneider, Early events in M-CSF receptor signaling. Growth Factors, 2000. 17(3): p. 155-66.
Pollard, J. W., Role of colony-stimulating factor-1 in reproduction and development. Mol Reprod Dev, 1997. 46(1): p. 54-60; discussion 60-1.
Dai, X. M., et al., Targeted disruption of the mouse colony-stimulating factor 1 receptor gene results in osteopetrosis, mononuclear phagocyte deficiency, increased primitive progenitor cell frequencies, and reproductive defects. Blood, 2002. 99(1): p. 111-20.
Scholl, S. M., et al., Anti-colony-stimulating factor-1 antibody staining in primary breast adenocarcinomas correlates with marked inflammatory cell infiltrates and prognosis. J Natl Cancer Inst, 1994. 86(2): p. 120-6.
Kacinski, B. M., CSF-1 and its receptor in breast carcinomas and neoplasms of the female reproductive tract. Mol Reprod Dev, 1997. 46(1): p. 71-4.
Ngan, H. Y., et al., Proto-oncogenes and p53 protein expression in normal cervical stratified squamous epithelium and cervical intra-epithelial neoplasia. Eur J Cancer, 1999. 35(10): p. 1546-50.
Kirma, N., et al., Elevated expression of the oncogene c-fins and its ligand, the macrophage colony-stimulating factor-1, in cervical cancer and the role of transforming growth factor-beta1 in inducing c-fins expression. Cancer Res, 2007. 67(5): p. 1918-26.
Ridge, S. A., et al., EMS mutations in myelodysplastic, leukemic, and normal subjects. Proc Natl Acad Sci USA, 1990. 87(4): p. 1377-80.
Abu-Duhier, F. M., et al., Mutational analysis of class III receptor tyrosine kinases (C-KIT, C-FMS, FLT3) in idiopathic myelofibrosis. Br J Haematol, 2003. 120(3): p. 464-70.
Yang, D. H., et al., The relationship between point mutation and abnormal expression of c-fins oncogene in hepatocellular carcinoma. Hepatobiliary Pancreat Dis Int, 2004. 3(1): p. 86-9.
West, R. B., et al., A landscape effect in tenosynovial giant-cell tumor from activation of CSF1 expression by a translocation in a minority of tumor cells. Proc Natl Acad Sci USA, 2006. 103(3): p. 690-5.
Tanaka, S., et al., Macrophage colony-stimulating factor is indispensable for both proliferation and differentiation of osteoclast progenitors. J Clin Invest, 1993. 91(1): p. 257-63.
Choueiri, M. B., et al., The central role of osteoblasts in the metastasis of prostate cancer. Cancer Metastasis Rev, 2006. 25(4): p. 601-9.
Vessella, R. L. and E. Corey, Targeting factors involved in bone remodeling as treatment strategies in prostate cancer bone metastasis. Clin Cancer Res, 2006. 12(20 Pt 2): p. 6285s-6290s.
Bingle, L., N. J. Brown, and C. E. Lewis, The role of tumour-associated macrophages in tumour progression: implications for new anticancer therapies. J Pathol, 2002. 196(3): p. 254-65.
Pollard, J. W., Tumour-educated macrophages promote tumour progression and metastasis. Nat Rev Cancer, 2004. 4(1): p. 71-8.
Zins, K., et al., Colon Cancer Cell-Derived Tumor Necrosis Factor-{alpha} Mediates the Tumor Growth-Promoting Response in Macrophages by Up-regulating the Colony-Stimulating Factor-1 Pathway 10.1158/0008-5472.CAN-06-2295. Cancer Res, 2007. 67(3): p. 1038-1045.
Paulus, P., et al., Colony-Stimulating Factor-1 Antibody Reverses Chemoresistance in Human MCF-7 Breast Cancer Xenografts 10.1158/0008-5472.CAN-05-3523. Cancer Res, 2006. 66(8): p. 4349-4356.
Balkwill, F., K. A. Charles, and A. Mantovani, Smoldering and polarized inflammation in the initiation and promotion of malignant disease. Cancer Cell, 2005. 7(3): p. 211-7.
Mantovani, A., et al., The chemokine system in diverse forms of macrophage activation and polarization. Trends Immunol, 2004. 25(12): p. 677-86.
Balkwill, F., TNF-alpha in promotion and progression of cancer. Cancer Metastasis Rev, 2006. 25(3): p. 409-16.
Cohen, M. S., et al., Structural bioinformatics-based design of selective, irreversible kinase inhibitors. Science, 2005. 308(5726): p. 1318-21.
Rabello, D., et al., CSF1 gene associated with aggressive periodontitis in the Japanese population. Biochem Biophys Res Commun, 2006. 347(3): p. 791-6.
da Costa, C. E., et al., Presence of osteoclast-like multinucleated giant cells in the bone and nonostotic lesions of Langerhans cell histiocytosis. J Exp Med, 2005. 201(5): p. 687-93.
Cenci, S., et al., M-CSF neutralization and egr-1 deficiency prevent ovariectomy-induced bone loss. J Clin Invest, 2000. 105(9): p. 1279-87.
Roggia, C., et al., Role of TNF-alpha producing T-cells in bone loss induced by estrogen deficiency. Minerva Med, 2004. 95(2): p. 125-32.
Kitaura, H., et al., M-CSF mediates TNF-induced inflammatory osteolysis. J Clin Invest, 2005. 115(12): p. 3418-27.
Daroszewska, A. and S. H. Ralston, Mechanisms of disease: genetics of Paget's disease of bone and related disorders. Nat Clin Pract Rheumatol, 2006. 2(5): p. 270-7.
Lester, J. E., et al., Current management of treatment-induced bone loss in women with breast cancer treated in the United Kingdom. Br J Cancer, 2006. 94(1): p. 30-5.
Lester, J., et al., The causes and treatment of bone loss associated with carcinoma of the breast. Cancer Treat Rev, 2005. 31(2): p. 115-42.
Stoch, S. A., et al., Bone loss in men with prostate cancer treated with gonadotropin-releasing hormone agonists. J Clin Endocrinol Metab, 2001. 86(6): p. 2787-91.
Drees, P., et al., Mechanisms of disease: Molecular insights into aseptic loosening of orthopedic implants. Nat Clin Pract Rheumatol, 2007. 3(3): p. 165-71.
Guzman-Clark, J. R., et al., Barriers in the management of glucocorticoid-induced osteoporosis. Arthritis Rheum, 2007. 57(1): p. 140-6.
Feldstein, A. C., et al., Practice patterns in patients at risk for glucocorticoid-induced osteoporosis. Osteoporos Int, 2005. 16(12): p. 2168-74.
Ritchlin, C. T., et al., Mechanisms of TNF-alpha-and RANKL-mediated osteoclastogenesis and bone resorption in psoriatic arthritis. J Clin Invest, 2003. 111(6): p. 821-31.
Campbell, I. K., et al., The colony-stimulating factors and collagen-induced arthritis: exacerbation of disease by M-CSF and G-CSF and requirement for endogenous M-CSF. J Leukoc Biol, 2000. 68(1): p. 144-50.
Saitoh, T., et al., Clinical significance of increased plasma concentration of macrophage colony-stimulating factor in patients with angina pectoris. J Am Coll Cardiol, 2000. 35(3): p. 655-65.
Ikonomidis, I., et al., Increased circulating C-reactive protein and macrophage-colony stimulating factor are complementary predictors of long-term outcome in patients with chronic coronary artery disease. Eur Heart J, 2005. 26(16): p. 1618-24.
Murayama, T., et al., Intraperitoneal administration of anti-c-fins monoclonal antibody prevents initial events of atherogenesis but does not reduce the size of advanced lesions in apolipoprotein E-deficient mice. Circulation, 1999. 99(13): p. 1740-6.
Hao, A. J., S. T. Dheen, and E. A. Ling, Expression of macrophage colony-stimulating factor and its receptor in microglia activation is linked to teratogen-induced neuronal damage. Neuroscience, 2002. 112(4): p. 889-900.
Murphy, G. M., Jr., L. Yang, and B. Cordell, Macrophage colony-stimulating factor augments beta-amyloid-induced interleukin-1, interleukin-6, and nitric oxide production by microglial cells. J Biol Chem, 1998. 273(33): p. 20967-71.
Murphy, G. M., Jr., et al., Expression of macrophage colony-stimulating factor receptor is increased in the AbetaPP(V717F) transgenic mouse model of Alzheimer's disease. Am J Pathol, 2000. 157(3): p. 895-904.
Kaku, M., et al., Amyloid beta protein deposition and neuron loss in osteopetrotic (op/op) mice. Brain Res Brain Res Protoc, 2003. 12(2): p. 104-8.

1.-66. (canceled)

67. A compound of Formula (III): 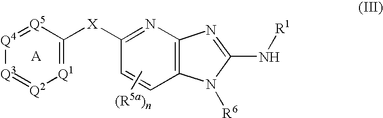
or a pharmaceutically acceptable salt, or solvate thereof,
wherein:
A is a six-member ring where each of Q¹, Q², Q³, Q⁴ and Q⁵ is independently C—R³ or N, provided that at least one of Q¹, Q², Q³, Q⁴ and Q⁵ is N and at most three of Q¹, Q², Q³, Q⁴ and Q⁵ are N;
each R³ is independently hydrogen or R^{3a}, where R^{3a} is selected from the group consisting of halo, alkyl, substituted alkyl, alkenyl, substituted alkenyl, alkynyl, substituted alkynyl, carbonitrile, aryl, substituted aryl, cycloalkyl, substituted cycloalkyl, cycloalkenyl, substituted cycloalkenyl, heteroaryl, substituted heteroaryl, heterocyclic, substituted heterocyclic, amino, substituted amino, acylamino, alkoxy, substituted alkoxy, carboxyl, carboxyl ester, substituted sulfonyl, aminosulfonyl, and aminocarbonyl; or two adjacent R^{3a} groups together form a aryl, substituted aryl, heterocyclic, substituted heterocyclic, heteroaryl, or substituted heteroaryl group that is fused to ring A;
R¹ is selected from the group consisting of hydrogen, alkyl, substituted alkyl, cycloalkyl, substituted cycloalkyl, cycloalkenyl, substituted cycloalkenyl, aryl, substituted aryl, heterocyclyl, substituted heterocyclyl, heteroaryl, substituted heteroaryl, acyl, and aminocarbonyl, or R¹ and R² are taken together to form a group selected from heterocyclyl, substituted heterocyclyl, heteroaryl, and substituted heteroaryl; provided R¹ and R² are not both hydrogen;
R^{5a} is independently selected from the group consisting of alkyl, substituted alkyl, alkoxy, substituted alkoxy, amino, substituted amino, and halo, or optionally when m is at least 2, two R^{5a} together with the carbon atom to which they are both attached from a C═O or C═S group;
R⁶ is independently selected from the group consisting of hydrogen, alkyl, and substituted alkyl;
X is selected from the group consisting of O, S, S(O), S(O)₂, and N—R⁴, wherein R⁴ is hydrogen, alkyl, or substituted alkyl; and
n is 0, 1, or 2.

68. A compound of Formula (IV): 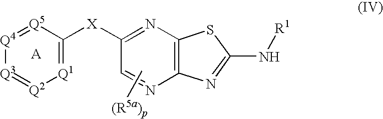
or a pharmaceutically acceptable salt, or solvate thereof,
wherein:
A is a six-member ring where each of Q¹, Q², Q³, Q⁴ and Q⁵ is independently C—R³ or N, provided that at least one of Q¹, Q², Q³, Q⁴ and Q⁵ is N and at most three of Q¹, Q², Q³, Q⁴ and Q⁵ are N;
each R³ is independently hydrogen or R^{3a}, where R^{3a} is selected from the group consisting of halo, alkyl, substituted alkyl, alkenyl, substituted alkenyl, alkynyl, substituted alkynyl, carbonitrile, aryl, substituted aryl, cycloalkyl, substituted cycloalkyl, cycloalkenyl, substituted cycloalkenyl, heteroaryl, substituted heteroaryl, heterocyclic, substituted heterocyclic, amino, substituted amino, acylamino, alkoxy, substituted alkoxy, carboxyl, carboxyl ester, substituted sulfonyl, aminosulfonyl, and aminocarbonyl; or two adjacent R^{3a} groups together form a aryl, substituted aryl, heterocyclic, substituted heterocyclic, heteroaryl, or substituted heteroaryl group that is fused to ring A;
R¹ is selected from the group consisting of hydrogen, alkyl, substituted alkyl, cycloalkyl, substituted cycloalkyl, cycloalkenyl, substituted cycloalkenyl, aryl, substituted aryl, heterocyclyl, substituted heterocyclyl, heteroaryl, substituted heteroaryl, acyl, and aminocarbonyl, or R¹ and R² are taken together to form a group selected from heterocyclyl, substituted heterocyclyl, heteroaryl, and substituted heteroaryl; provided R¹ and R² are not both hydrogen;
R^{5a} is independently selected from the group consisting of alkyl, substituted alkyl, alkoxy, substituted alkoxy, amino, substituted amino, and halo, or optionally when m is at least 2, two R^{5a} together with the carbon atom to which they are both attached from a C═O or C═S group;
X is selected from the group consisting of O, S, S(O), S(O)₂, and N—R⁴, wherein R⁴ is hydrogen, alkyl, or substituted alkyl; and
p is 0, or 1.

69. A compound of Formula (V): 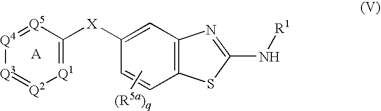
or a pharmaceutically acceptable salt, or solvate thereof,
wherein:
A is a six-member ring where each of Q¹, Q², Q³, Q⁴ and Q⁵ is independently C—R³ or N, provided that at least one of Q¹, Q², Q³, Q⁴ and Q⁵ is N and at most three of Q¹, Q², Q³, Q⁴ and Q⁵ are N;
each R³ is independently hydrogen or R^{3a}, where R^{3a} is selected from the group consisting of halo, alkyl, substituted alkyl, alkenyl, substituted alkenyl, alkynyl, substituted alkynyl, carbonitrile, aryl, substituted aryl, cycloalkyl, substituted cycloalkyl, cycloalkenyl, substituted cycloalkenyl, heteroaryl, substituted heteroaryl, heterocyclic, substituted heterocyclic, amino, substituted amino, acylamino, alkoxy, substituted alkoxy, carboxyl, carboxyl ester, substituted sulfonyl, aminosulfonyl, and aminocarbonyl; or two adjacent R^{3a} groups together form a aryl, substituted aryl, heterocyclic, substituted heterocyclic, heteroaryl, or substituted heteroaryl group that is fused to ring A;
R¹ is selected from the group consisting of hydrogen, alkyl, substituted alkyl, cycloalkyl, substituted cycloalkyl, cycloalkenyl, substituted cycloalkenyl, aryl, substituted aryl, heterocyclyl, substituted heterocyclyl, heteroaryl, substituted heteroaryl, acyl, and aminocarbonyl, or R¹ and R² are taken together to form a group selected from heterocyclyl, substituted heterocyclyl, heteroaryl, and substituted heteroaryl; provided R¹ and R² are not both hydrogen;
R^{5a} is independently selected from the group consisting of alkyl, substituted alkyl, alkoxy, substituted alkoxy, amino, substituted amino, and halo, or optionally when m is at least 2, two R^{5a} together with the carbon atom to which they are both attached from a C═O or C═S group;
X is selected from the group consisting of O, S, S(O), S(O)₂, and N—R⁴, wherein R⁴ is hydrogen, alkyl, or substituted alkyl; and
q is 0, 1, 2 or 3.

70. A compound of claim 67, 68 or 69 selected from:

| Structure | Name |
|---|---|
| 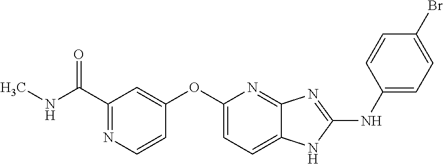 | 3-(2-(4-bromophenylamino)- 1H-imidazo[4,5-b]pyridin-5- yloxy)-N-methylbenzamide |
| 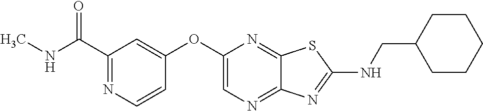 | 3-(2-(cyclohexylmethylamino) thiazolo[4,5-b]pyrazin-6- yloxy)-N-methylbenzamide |
| 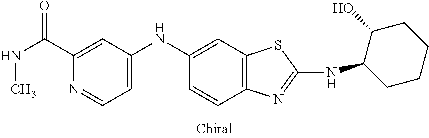 | 4-(2-((1R,2R)-2-hydroxy- cyclohexylamino)benzo[d] thiazol-6-ylamino)-N- methylpicolinamide |
| 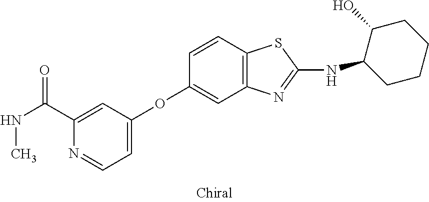 | 4-(2-((1R,2R)-2-hydroxy- cyclohexylamino)benzo[d] thiazol-5-yloxy)-N-methyl- picolinamide |

71. A compound of Formula (VII): 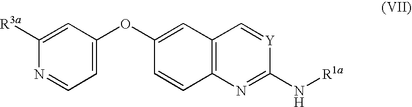
or an oxide, ester, prodrug, pharmaceutically acceptable salt, or solvate thereof,
wherein:
Y is N or CH;
R^{1a} is selected from the group consisting of hydrogen, alkyl, substituted alkyl, cycloalkyl, substituted cycloalkyl, cycloalkenyl, substituted cycloalkenyl, heterocyclyl, substituted heterocyclyl, acyl, and aminocarbonyl; and
R^{1a} is selected from the group consisting of halo, alkyl, substituted alkyl, alkenyl, substituted alkenyl, alkynyl, substituted alkynyl, carbonitrile, aryl, substituted aryl, cycloalkyl, substituted cycloalkyl, cycloalkenyl, substituted cycloalkenyl, heteroaryl, substituted heteroaryl, heterocyclic, substituted heterocyclic, amino, substituted amino, acylamino, alkoxy, substituted alkoxy, carboxyl, carboxyl ester, substituted sulfonyl, aminosulfonyl, and aminocarbonyl:
provided that R^{1a} is not:

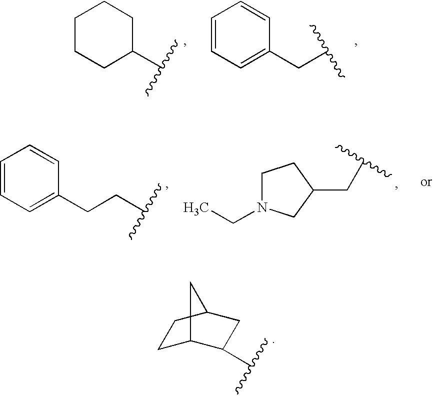

72. A compound of claim 71, wherein R^{1a} is cycloalkyl, substituted cycloalkyl, heterocyclyl or substituted heterocyclyl.

73. A compound of claim 72, wherein R^{1a} is cyclohexyl or cyclopentyl, wherein said cyclohexyl and cyclopentyl are optionally substituted with one to four substituents independently selected from the group consisting of hydroxy and amino; or two adjacent substituents join together to form a benzene ring fused with the cyclohexyl or cyclopentyl.

74. A compound of claim 72, wherein R^{1a} is selected from the group consisting of:

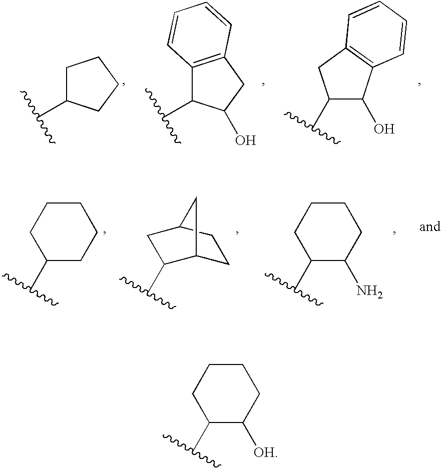

75. A compound of claim 71, wherein R^{1a} is tetrahydropyran, piperidinyl or substituted piperidinyl.

76. A compound of claim 71, wherein R^{1a} is alkyl or substituted alkyl.

77. A compound of claim 76, wherein R^{1a} is alkyl substituted with one to four substituents selected from the group consisting of cycloalkyl, substituted cycloalkyl, hydroxy, phenyl, substituted phenyl, heterocyclyl, substituted heterocyclyl, heteroaryl and substituted heteroaryl.

78. A compound of claim 76, wherein R^{1a} is alkyl substituted with at least one substituent selected from the group consisting of hydroxyl, cyclopropyl, cyclohexyl, morpholino, phenyl, substituted phenyl, thiazole and substituted thiazole.

79. A compound of claim 71, wherein R^{1a} is acyl.

80. A compound of claim 71, wherein R^{1a} is —CO—R⁸ or —CO—NH—R⁸, wherein R⁸ is optionally substituted phenyl or optionally substituted cyclohexyl.

81. A compound of claim 71, wherein R^{3a} is selected from hydrogen, aryl, substituted aryl, cycloalkyl, substituted cycloalkyl, cycloalkenyl, substituted cycloalkenyl, heteroaryl, substituted heteroaryl, heterocyclic, substituted heterocyclic, amino, substituted amino, acylamino, alkoxy, substituted alkoxy, carboxyl, carboxyl ester, substituted sulfonyl, aminosulfonyl, and aminocarbonyl.

82. A compound of claim 71, wherein R^{3a} is selected from the group consisting of hydrogen, pyrazolyl, substituted pyrazolyl, imidazolyl, substituted imidazolyl, pyridinyl, substituted pyridinyl, acylamino and aminocarbonyl.

83. A compound of claim 71, wherein R^{3a} group is independently selected from the group consisting of hydrogen,

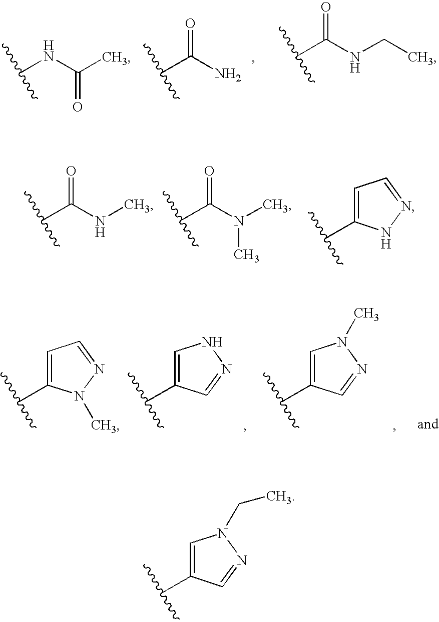

84. A compound of claim 71 selected from:

| Structure | Name |
|---|---|
| 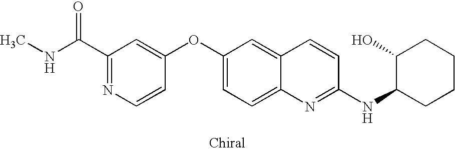 | 4-(2-((1R,2R)-2- hydroxycyclohexylamino) quinolin-6-yloxy)-N- methylpicolinamide |
| 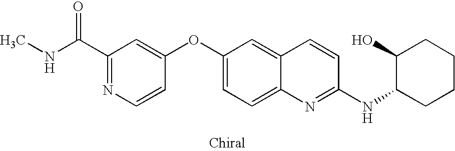 | 4-(2-((1S,2S)-2- hydroxycyclohexylamino) quinolin-6-yloxy)-N- methylpicolinamide |
| 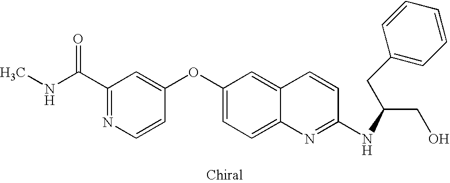 | (S)-4-(2-(1-hydroxy-3- phenylpropan-2- ylamino)quinolin-6-yloxy)-N- methylpicolinamide |
| 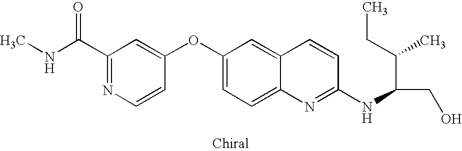 | 4-(2-((2S,3S)-1-hydroxy-3- methylpentan-2- ylamino)quinolin-6-yloxy)-N- methylpicolinamide |
| 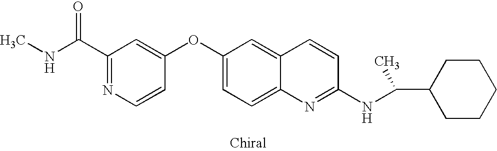 | (R)-4-(2-(1- cyclohexylethylamino)quinolin- 6-yloxy)-N- methylpicolinamide |
| 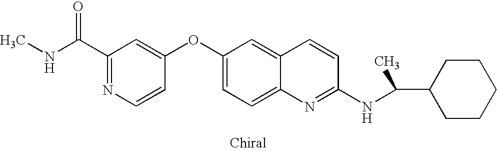 | (S)-4-(2-(1- cyclohexylethylamino)quinolin- 6-yloxy)-N- methylpicolinamide |
| 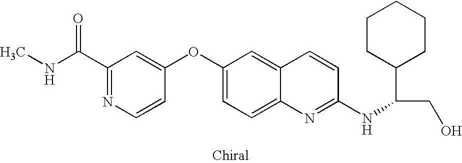 | (R)-4-(2-(1-cyclohexyl-2- hydroxyethylamino)quinolin-6- yloxy)-N-methylpicolinamide |
| 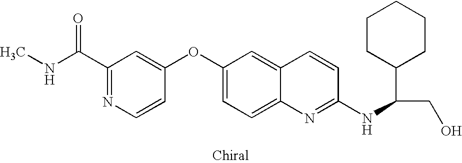 | (S)-4-(2-(1-cyclohexyl-2- hydroxyethylamino)quinolin-6- yloxy)-N-methylpicolinamide |
| 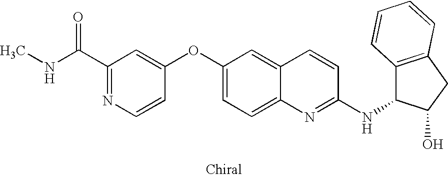 | 4-(2-((1R,2S)-2-hydroxy-2,3- dihydro-1H-inden-1- ylamino)quinolin-6-yloxy)-N- methylpicolinamide |
| 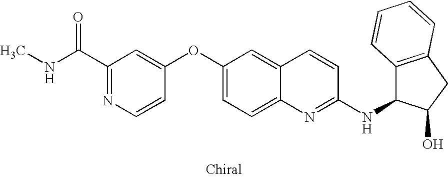 | 4-(2-((1S,2R)-2-hydroxy-2,3- dihydro-1H-inden-1- ylamino)quinolin-6-yloxy)-N- methylpicolinamide |
| 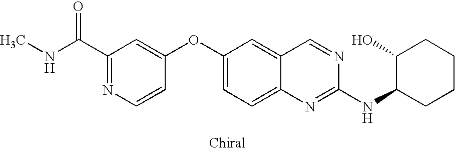 | 4-(2-((1R,2R)-2- hydroxycyclohexylamino) quinazolin-6-yloxy)-N- methylpicolinamide |
| 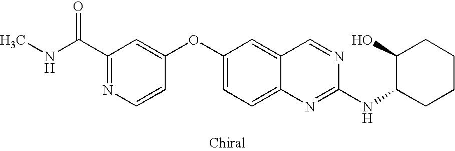 | 4-(2-((1S,2S)-2- hydroxycyclohexylamino) quinazolin-6-yloxy)-N- methylpicolinamide |
| 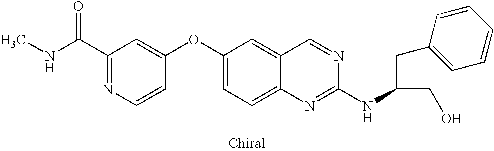 | (S)-4-(2-(1-hydroxy-3- phenylpropan-2- ylamino)quinazolin-6-yloxy)- N-methylpicolinamide |
| 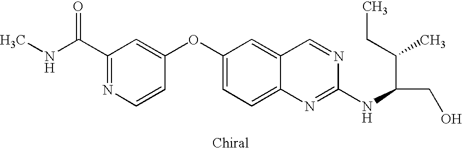 | 4-(2-((2S,3S)-1-hydroxy-3- methylpentan-2- ylamino)quinazolin-6-yloxy)- N-methylpicolinamide |
| 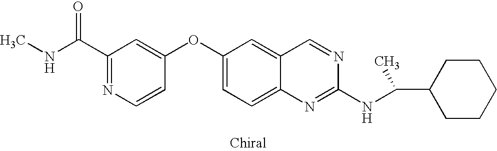 | (R)-4-(2-(1- cyclohexylethylamino)quinazolin- 6-yloxy)-N- methylpicolinamide |
| 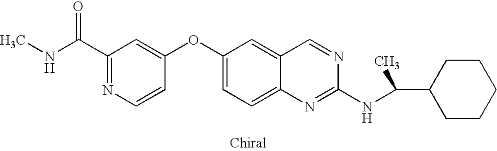 | (S)-4-(2-(1- cyclohexylethylamino)quinazolin- 6-yloxy)-N- methylpicolinamide |
| 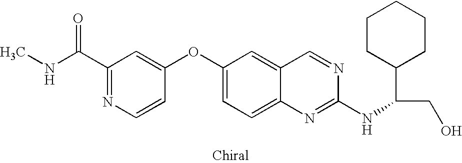 | (R)-4-(2-(1-cyclohexyl-2- hydroxyethylamino)quinazolin- 6-yloxy)-N- methylpicolinamide |
| 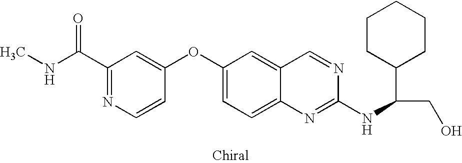 | (S)-4-(2-(1-cyclohexyl-2- hydroxyethylamino)quinazolin- 6-yloxy)-N- methylpicolinamide |
| 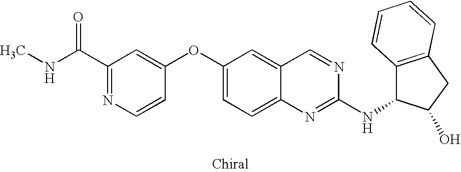 | 4-(2-((1R,2S)-2-hydroxy-2,3- dihydro-1H-inden-1- ylamino)quinazolin-6-yloxy)- N-methylpicolinamide |
| 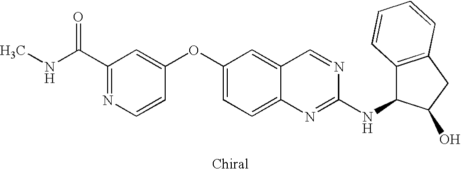 | 4-(2-((1S,2R)-2-hydroxy-2,3- dihydro-1H-inden-1- ylamino)quinazolin-6-yloxy)- N-methylpicolinamide |
| 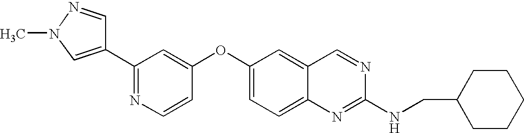 | N-(cyclohexylmethyl)-6-(2-(1- methyl-1H-pyrazol-4- yl)pyridin-4-yloxy)quinazolin- 2-amine |
| 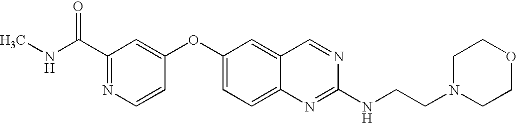 | N-methyl-4-(2-(2- morpholinoethylamino) quinazolin- 6-yloxy)picolinamide |
| 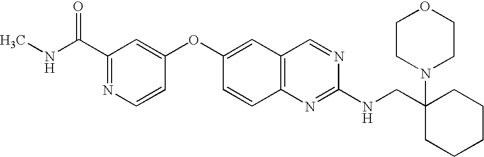 | N-methyl-4-(2-((1- morpholinocyclohexyl) methylamino)quinazolin-6- yloxy)picolinamide |
| 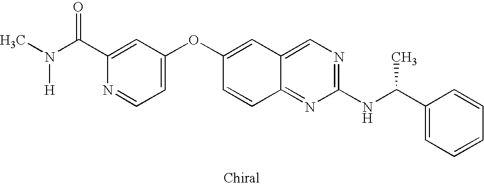 | (R)-N-methyl-4-(2-(1- phenylethylamino)quinazolin- 6-yloxy)picolinamide |
| 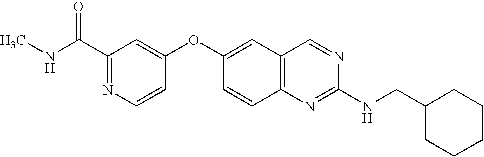 | 4-(2- (cyclohexylmethylamino) quinazolin-6-yloxy)-N- methylpicolinamide |
| 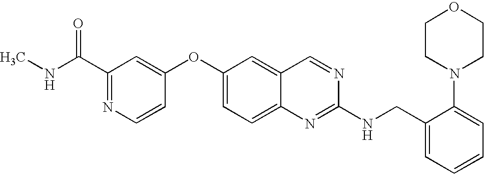 | N-methyl-4-(2-(2- morpholinobenzylamino) quinazolin-6-yloxy)picolinamide |
| 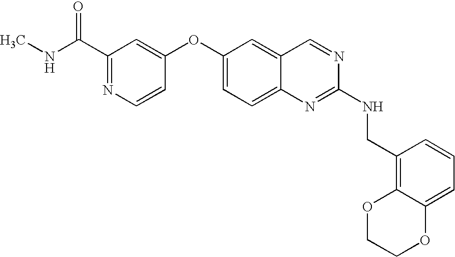 | 4-(2-((2,3- dihydrobenzo[b][1,4]dioxin-5- yl)methylamino)quinazolin-6- yloxy)-N-methylpicolinamide |
| 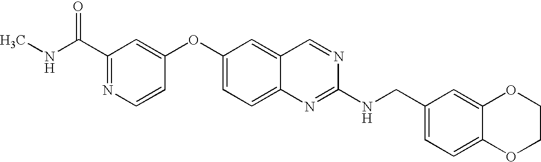 | 4-(2-((2,3- dihydrobenzo[b][1,4]dioxin-6- yl)methylamino)quinazolin-6- yloxy)-N-methylpicolinamide |
| 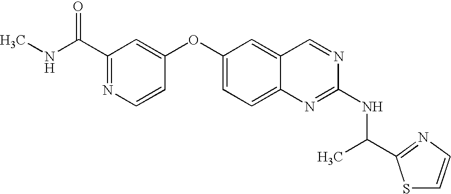 | N-methyl-4-(2-(1-(thiazol-2- yl)ethylamino)quinazolin-6- yloxy)picolinamide |
| 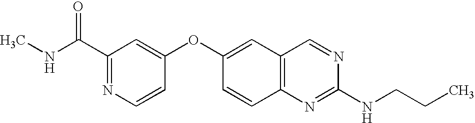 | N-methyl-4-(2- (propylamino)quinazolin-6- yloxy)picolinamide |
| 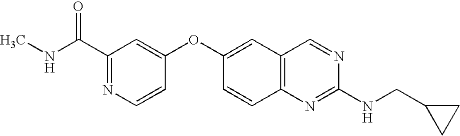 | 4-(2- (cyclopropylmethylamino) quinazolin-6-yloxy)-N- methylpicolinamide |
| 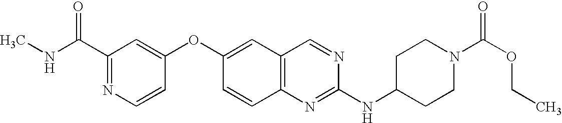 | ethyl 4-(6-(2- (methylcarbamoyl)pyridin-4- yloxy)quinazolin-2- ylamino)piperidine-1- carboxylate |
| 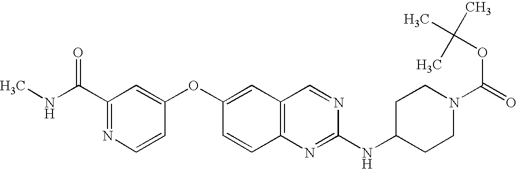 | tert-butyl 4-(6-(2- (methylcarbamoyl)pyridin-4- yloxy)quinazolin-2- ylamino)piperidine-1- carboxylate |
| 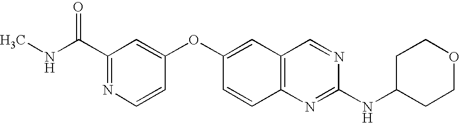 | N-methyl-4-(2-(tetrahydro-2H- pyran-4-ylamino)quinazolin-6- yloxy)picolinamide |
| 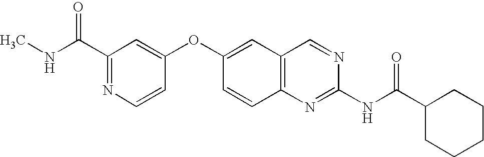 | 4-(2- (cyclohexanecarboxamido) quinazolin-6-yloxy)-N- methylpicolinamide |
| 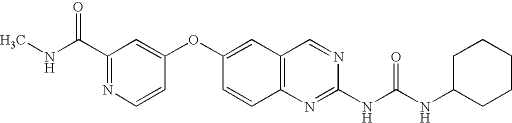 | 4-(2-(3- cyclohexylureido)quinazolin-6- yloxy)-N-methylpicolinamide |
| 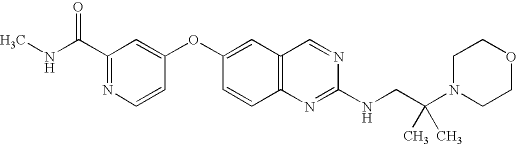 | N-methyl-4-(2-(2-methyl-2- morpholinopropylamino) quinazolin-6-yloxy)picolinamide |
| 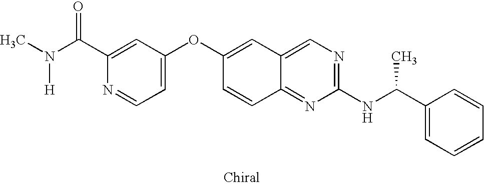 | (R)-N-methyl-4-(2-(1- phenylethylamino)quinazolin- 6-yloxy)picolinamide |
| 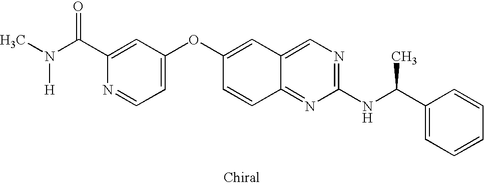 | (S)-N-methyl-4-(2-(1- phenylethylamino)quinazolin- 6-yloxy)picolinamide |
| 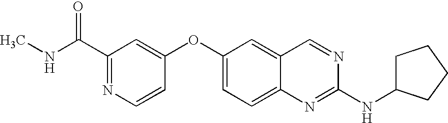 | 4-(2- (cyclopentylamino)quinazolin- 6-yloxy)-N- methylpicolinamide |
| 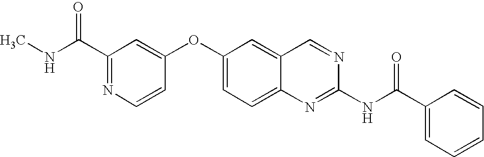 | 4-(2-benzamidoquinazolin-6- yloxy)-N-methylpicolinamide |
| 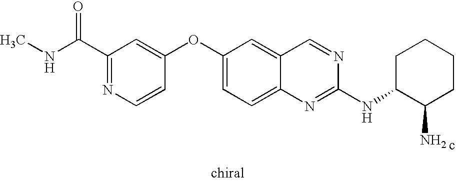 | 4-(2-((1R,2R)-2- aminocyclohexylamino) quinazolin-6-yloxy)-N- methylpicolinamide |
| 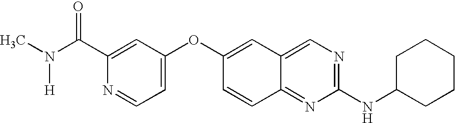 | 4-(2- (cyclohexylamino)quinazolin- 6-yloxy)-N- methylpicolinamide |

or a pharmaceutically acceptable salt, or solvate thereof.

85. A compound selected from:

| Structure | Name |
|---|---|
| 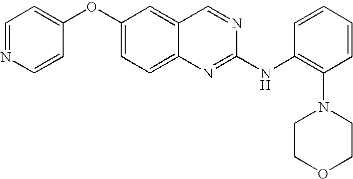 | N-(2-morpholinophenyl)-6- (pyridin-4-yloxy)quinazolin-2- amine |
| 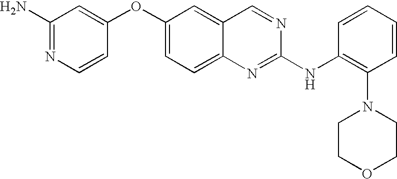 | 6-(2-aminopyridin-4-yloxy)- N-(2- morpholinophenyl)quinazolin- 2-amine |
| 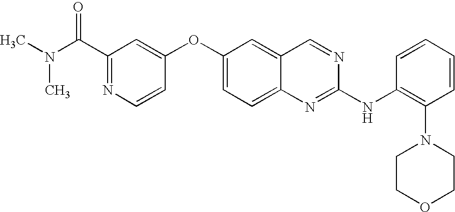 | N,N-dimethyl-4-(2-(2- morpholinophenylamino) quinazolin-6-yloxy)picolinamide |
| 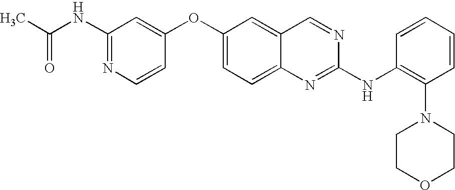 | N-(4-(2-(2- morpholinophenylamino) quinazolin-6-yloxy)pyridin-2- yl)acetamide |
| 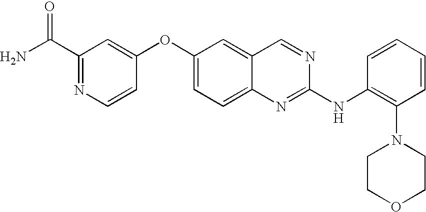 | 4-(2-(2- morpholinophenylamino) quinazolin-6-yloxy)picolinamide |
| 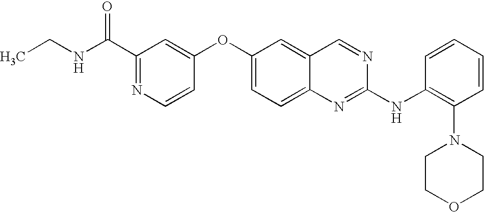 | N-ethyl-4-(2-(2- morpholinophenylamino) quinazolin-6-yloxy)picolinamide |
| 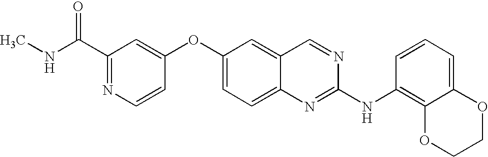 | 4-(2-(2,3- dihydrobenzo[b][1,4]dioxin-5- ylamino)quinazolin-6-yloxy)- N-methylpicolinamide |
| 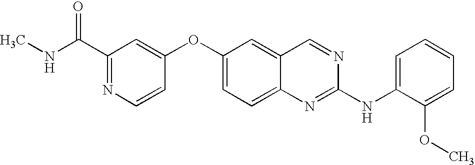 | 4-(2-(2- methoxyphenylamino) quinazolin-6-yloxy)-N- methylpicolinamide |
| 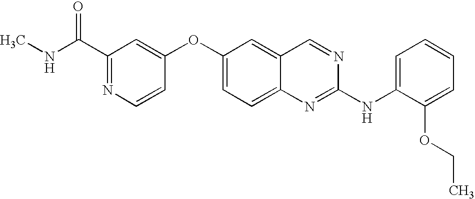 | 4-(2-(2- ethoxyphenylamino) quinazolin-6-yloxy)-N- methylpicolinamide |
| 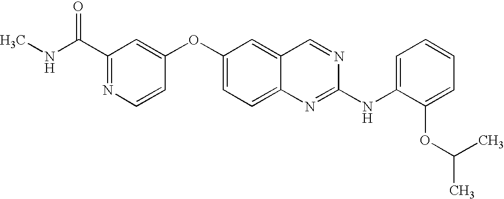 | 4-(2-(2- isopropoxyphenylamino) quinazolin-6-yloxy)-N- methylpicolinamide |
| 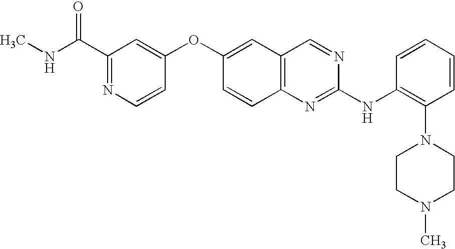 | N-methyl-4-(2-(2-(4- methylpiperazin-1- yl)phenylamino)quinazolin-6- yloxy)picolinamide |
| 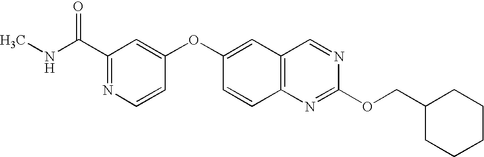 | 4-(2- (cyclohexylmethoxy) quinazolin-6-yloxy)-N- methylpicolinamide |
| 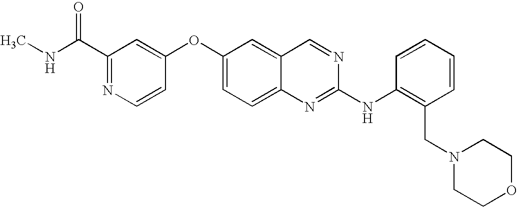 | N-methyl-4-(2-(2- (morpholinomethyl) phenylamino)quinazolin-6- yloxy)picolinamide |
| 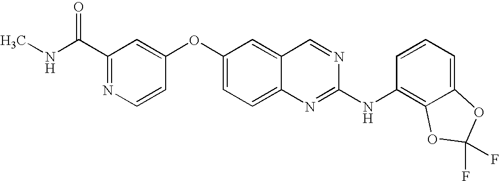 | 4-(2-(2,2- difluorobenzo[d][1,3]dioxol-4- ylamino)quinazolin-6-yloxy)- N-methylpicolinamide |
| 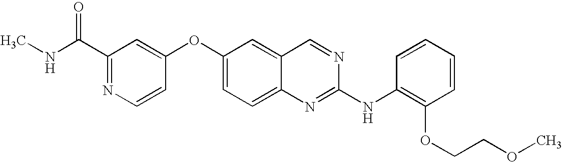 | 4-(2-(2-(2- methoxyethoxy)phenylamino) quinazolin-6-yloxy)-N- methylpicolinamide |
| 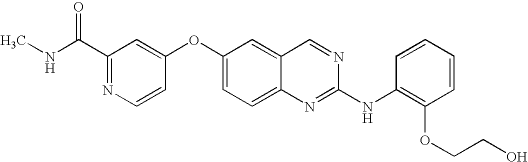 | 4-(2-(2-(2- hydroxyethoxy)phenylamino) quinazolin-6-yloxy)-N- methylpicolinamide |
| 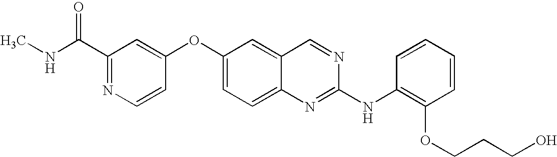 | 4-(2-(2-(3- hydroxypropoxy)phenylamino) quinazolin-6-yloxy)-N- methylpicolinamide |
| 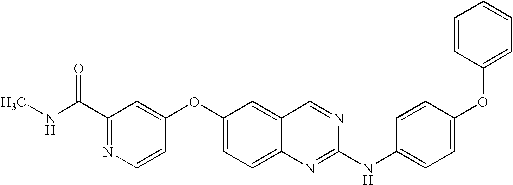 | N-methyl-4-(2-(4- phenoxyphenylamino) quinazolin-6-yloxy)picolinamide |
| 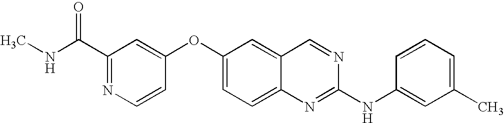 | N-methyl-4-(2-(m- tolylamino)quinazolin-6- yloxy)picolinamide |

or an oxide, ester, prodrug, pharmaceutically acceptable salt, or solvate thereof.

86. A compound selected from:

| Structure | Name |
|---|---|
| 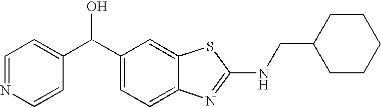 | (2- (cyclohexylmethylamino)benzo [d]thiazol-6-yl)(pyridin-4- yl)methanol |
| 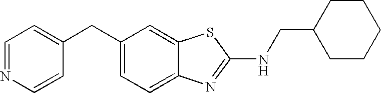 | N-(cyclohexylmethyl)-6- (pyridin-4- ylmethyl)benzo[d]thiazol-2- amine |
| 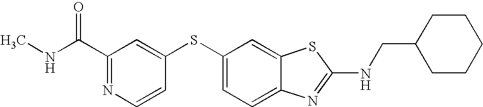 | 4-(2- (cyclohexylmethylamino)benzo [d]thiazol-6-ylthio)-N- methylpicolinamide |
| 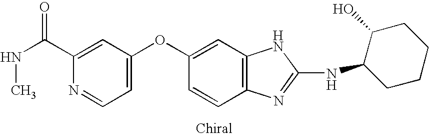 | 4-(2-((1R,2R)-2- hydroxycyclohexylamino)-1H- benzo[d]imidazol-6-yloxy)-N- methylpicolinamide |
| 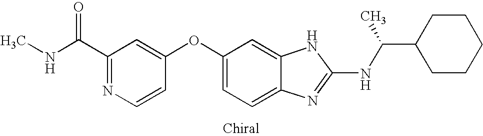 | (R)-4-(2-(1- cyclohexylethylamino)-1H- benzo[d]imidazol-6-yloxy)-N- methylpicolinamide |

or a pharmaceutically acceptable salt, or solvate thereof.

87. A pharmaceutical composition effective to inhibit CSF-1R activity in a patient when administered thereto, comprising a therapeutically effective amount of a compound of any one of claims 67-86 and a pharmaceutically acceptable carrier.

88. A composition of claim 87, wherein said compound exhibits an IC₅₀ value with respect to CSF-1R inhibition of less than 1 μM.

89. A composition of claim 88, further comprising an additional agent.

90. A composition of claim 89, wherein said additional agent is a bisphosphonate.

91. A method of treating a CSF-1R mediated disorder in a patient, comprising administering to the patient a compound of any one of claims 67-86 effective to inhibit CSF-1R activity in the patient.

92. A method of claim 90, wherein the CSF-1R mediated disorder is selected from the group consisting of cancer, osteoporosis, arthritis, atherosclerosis and chronic glomerular nephritis.

93. A method of claim 90, wherein the CSF-1R mediated disorder is a cancer selected from the group consisting of myelocytic leukemia, idiopathic myelofibrosis, breast cancer, cervical cancer, ovarian cancer, endometrial cancer, prostate cancer, hepatocellular cancer, multiple myeloma, lung cancer, renal cancer, and bone cancer.

94. A method of claim 93, wherein the CSF-1R mediated disorder is rheumatoid arthritis.
